SEQUENCE LISTING (173 sequences)

SEQ ID NO: 1 - DNA, 10 bp, Artificial Sequence
  source: /note=“Description of Artificial Sequence Synthetic oligonucleotide”
gaaanngaaa

SEQ ID NO: 2 - DNA, 10 bp, Artificial Sequence
  source: /note=“Description of Artificial Sequence Synthetic oligonucleotide”
ggaanngaaa

SEQ ID NO: 3 - DNA, 15 bp, Artificial Sequence
  source: /note=“Description of Artificial Sequence Synthetic oligonucleotide”
tttcnnnntgastma

SEQ ID NO: 4 - DNA, 11 bp, Artificial Sequence
  source: /note=“Description of Artificial Sequence Synthetic oligonucleotide”
gaaatgastma

SEQ ID NO: 5 - DNA, 24 bp, Artificial Sequence
  source: /note=“Description of Artificial Sequence Synthetic primer”
tgtttgtgtgcttctgagcagggt

SEQ ID NO: 6 - DNA, 23 bp, Artificial Sequence
  source: /note=“Description of Artificial Sequence Synthetic primer”
gggttgcccagagactgctgtgt

SEQ ID NO: 7 - DNA, 25 bp, Artificial Sequence
  source: /note=“Description of Artificial Sequence Synthetic primer”
acatgagtgccgacaaacaacgggt

SEQ ID NO: 8 - DNA, 25 bp, Artificial Sequence
  source: /note=“Description of Artificial Sequence Synthetic primer”
agtcagtgggctgctgaatgacccc

SEQ ID NO: 9 - DNA, 23 bp, Artificial Sequence
  source: /note=“Description of Artificial Sequence Synthetic primer”
gcacaggaggcaccgtcagattt

SEQ ID NO: 10 - DNA, 22 bp, Artificial Sequence
  source: /note=“Description of Artificial Sequence Synthetic primer”
ggccatgccgctgctttactca

SEQ ID NO: 11 - DNA, 22 bp, Artificial Sequence
  source: /note=“Description of Artificial Sequence Synthetic primer”
ccaacctgtgcacttggccacc

SEQ ID NO: 12 - DNA, 21 bp, Artificial Sequence
  source: /note=“Description of Artificial Sequence Synthetic primer”
ggaaggccggcactttgccct

SEQ ID NO: 13 - DNA, 25 bp, Artificial Sequence
  source: /note=“Description of Artificial Sequence Synthetic primer”
ggaaggaagtttgccaagaacccag

SEQ ID NO: 14 - DNA, 25 bp, Artificial Sequence
  source: /note=“Description of Artificial Sequence Synthetic primer”
ttatgagtgtgcaaacccaactgcc

SEQ ID NO: 15 - DNA, 20 bp, Artificial Sequence
  source: /note=“Description of Artificial Sequence Synthetic primer”
gcagaggggcccccaccttt

SEQ ID NO: 16 - DNA, 20 bp, Artificial Sequence
  source: /note=“Description of Artificial Sequence Synthetic primer”
ccgccttgtccacgctcaga

SEQ ID NO: 17 - DNA, 26 bp, Artificial Sequence
  source: /note=“Description of Artificial Sequence Synthetic primer”
gcatttgcttcaggcttttacctatg

SEQ ID NO: 18 - DNA, 25 bp, Artificial Sequence
  source: /note=“Description of Artificial Sequence Synthetic primer”
ggcagctcactttcagtaatctggg

SEQ ID NO: 19 - DNA, 20 bp, Artificial Sequence
  source: /note=“Description of Artificial Sequence Synthetic primer”
cagggccagggtcttcgggt

SEQ ID NO: 20 - DNA, 23 bp, Artificial Sequence
  source: /note=“Description of Artificial Sequence Synthetic primer”
agcaggtgctgggtaagtgtcag

SEQ ID NO: 21 - DNA, 31 bp, Artificial Sequence
  source: /note=“Description of Artificial Sequence Synthetic probe”
tagtgcagaaatgagtcagagatcaaagaag

SEQ ID NO: 22 - DNA, 31 bp, Artificial Sequence
  source: /note=“Description of Artificial Sequence Synthetic probe”
tagtgcatacatgagtcagagatcaaagaag

SEQ ID NO: 23 - DNA, 31 bp, Artificial Sequence
  source: /note=“Description of Artificial Sequence Synthetic probe”
tagtgcagaaaggcgtcagagatcaaagaag

SEQ ID NO: 24 - DNA, 31 bp, Artificial Sequence
  source: /note=“Description of Artificial Sequence Synthetic probe”
tagtgcatacaggcgtcagagatcaaagaag

SEQ ID NO: 25 - DNA, 40 bp, Artificial Sequence
  source: /note=“Description of Artificial Sequence Synthetic probe”
cttgccttagaggtttcgggatgactaatactgtacgtga

SEQ ID NO: 26 - DNA, 40 bp, Artificial Sequence
  source: /note=“Description of Artificial Sequence Synthetic probe”
cttgccttagaggtgtagggatgactaatactgtacgtga

SEQ ID NO: 27 - DNA, 40 bp, Artificial Sequence
  source: /note=“Description of Artificial Sequence Synthetic probe”
cttgccttagaggtttcgggaggcctaatactgtacgtga

SEQ ID NO: 28 - DNA, 40 bp, Artificial Sequence
  source: /note=“Description of Artificial Sequence Synthetic probe”
cttgccttagaggtgtagggaggcctaatactgtacgtga

SEQ ID NO: 29 - DNA, 40 bp, Artificial Sequence
  source: /note=“Description of Artificial Sequence Synthetic probe”
gcttttgctttcactttgactggcctggagacaatgagtt

SEQ ID NO: 30 - DNA, 40 bp, Artificial Sequence
  source: /note=“Description of Artificial Sequence Synthetic probe”
gcttttgctgtaactttgactggcctggagacaatgagtt

SEQ ID NO: 31 - DNA, 40 bp, Artificial Sequence
  source: /note=“Description of Artificial Sequence Synthetic probe”
gcttttgctttcacttggcctggcctggagacaatgagtt

SEQ ID NO: 32 - DNA, 40 bp, Artificial Sequence
  source: /note=“Description of Artificial Sequence Synthetic probe”
aaaggcacttctgcaatgagtaaagcagcggcatggcctt

SEQ ID NO: 33 - DNA, 40 bp, Artificial Sequence
  source: /note=“Description of Artificial Sequence Synthetic probe”
aaaggcacttcttcattgagtaaagcagcggcatggcctt

SEQ ID NO: 34 - DNA, 40 bp, Artificial Sequence
  source: /note=“Description of Artificial Sequence Synthetic probe”
aaaggcacttctgcaaggcgtaaagcagcggcatggcctt

SEQ ID NO: 35 - DNA, 40 bp, Artificial Sequence
  source: /note=“Description of Artificial Sequence Synthetic probe”
cgtgtccatttcataatgactagttgtcatgtcactattt

SEQ ID NO: 36 - DNA, 40 bp, Artificial Sequence
  source: /note=“Description of Artificial Sequence Synthetic probe”
cgtgtccatgtaataatgactagttgtcatgtcactattt

SEQ ID NO: 37 - DNA, 40 bp, Artificial Sequence
  source: /note=“Description of Artificial Sequence Synthetic probe”
cgtgtccatttcataaggcctagttgtcatgtcactattt

SEQ ID NO: 38 - DNA, 40 bp, Artificial Sequence
  source: /note=“Description of Artificial Sequence Synthetic probe”
aggtttacagaaggagtgactaagaacactagtgcataag

SEQ ID NO: 39 - DNA, 40 bp, Artificial Sequence
  source: /note=“Description of Artificial Sequence Synthetic probe”
aggtttacagaaggagtgactaatcatactagtgcataag

SEQ ID NO: 40 - DNA, 40 bp, Artificial Sequence
  source: /note=“Description of Artificial Sequence Synthetic probe”
aggtttacagaaggagtgacgacgaacactagtgcataag

SEQ ID NO: 41 - DNA, 40 bp, Artificial Sequence
  source: /note=“Description of Artificial Sequence Synthetic probe”
tggccaccgtgaatgttgactaataacaggagcccatcag

SEQ ID NO: 42 - DNA, 40 bp, Artificial Sequence
  source: /note=“Description of Artificial Sequence Synthetic probe”
tggccaccgtgaatgttgactaatcataggagcccatcag

SEQ ID NO: 43 - DNA, 40 bp, Artificial Sequence
  source: /note=“Description of Artificial Sequence Synthetic probe”
tggccaccgtgaatgtggactaataacaggagcccatcag

SEQ ID NO: 44 - DNA, 40 bp, Artificial Sequence
  source: /note=“Description of Artificial Sequence Synthetic probe”
cttgaacccaggagtttgagtaaattcttgcctggcagtt

SEQ ID NO: 45 - DNA, 40 bp, Artificial Sequence
  source: /note=“Description of Artificial Sequence Synthetic probe”
cttgaacccaggagtttgagtaatcatttgcctggcagtt

SEQ ID NO: 46 - DNA, 40 bp, Artificial Sequence
  source: /note=“Description of Artificial Sequence Synthetic probe”
cttgaacccaggagttggcgtaaattcttgcctggcagtt

SEQ ID NO: 47 - DNA, 40 bp, Artificial Sequence; identical to SEQ ID NO: 41
  source: /note=“Description of Artificial Sequence Synthetic probe”

SEQ ID NO: 48 - DNA, 40 bp, Artificial Sequence; identical to SEQ ID NO: 42
  source: /note=“Description of Artificial Sequence Synthetic probe”

SEQ ID NO: 49 - DNA, 40 bp, Artificial Sequence; identical to SEQ ID NO: 43
  source: /note=“Description of Artificial Sequence Synthetic probe”

SEQ ID NO: 50 - DNA, 24 bp, Artificial Sequence
  source: /note=“Description of Artificial Sequence Synthetic primer”
cagcgtgtccaaacactgaggcca

SEQ ID NO: 51 - DNA, 24 bp, Artificial Sequence
  source: /note=“Description of Artificial Sequence Synthetic primer”
cattgcggtggagagtccagggtg

SEQ ID NO: 52 - DNA, 23 bp, Artificial Sequence
  source: /note=“Description of Artificial Sequence Synthetic primer”
acccaccgccatactttgtgggc

SEQ ID NO: 53 - DNA, 25 bp, Artificial Sequence
  source: /note=“Description of Artificial Sequence Synthetic primer”
tccgggcatggttctggatcaatga

SEQ ID NO: 54 - DNA, 20 bp, Artificial Sequence
  source: /note=“Description of Artificial Sequence Synthetic primer”
gcagtacagccccaaaatgg

SEQ ID NO: 55 - DNA, 23 bp, Artificial Sequence
  source: /note=“Description of Artificial Sequence Synthetic primer”
aacaaagtctggcctgtatccaa

SEQ ID NO: 56 - DNA, 20 bp, Artificial Sequence
  source: /note=“Description of Artificial Sequence Synthetic primer”
gtgcgggtgagcgcacaagc

SEQ ID NO: 57 - DNA, 20 bp, Artificial Sequence
  source: /note=“Description of Artificial Sequence Synthetic primer”
cagcccggggtacttgccgc

SEQ ID NO: 58 - DNA, 21 bp, Artificial Sequence
  source: /note=“Description of Artificial Sequence Synthetic primer”
agcccccaagggccagatcgc

SEQ ID NO: 59 - DNA, 25 bp, Artificial Sequence
  source: /note=“Description of Artificial Sequence Synthetic primer”
agctgcatgctcacagtgccccttt

SEQ ID NO: 60 - DNA, 23 bp, Artificial Sequence
  source: /note=“Description of Artificial Sequence Synthetic primer”
gctcctgtcacatcagcggtgac

SEQ ID NO: 61 - DNA, 23 bp, Artificial Sequence
  source: /note=“Description of Artificial Sequence Synthetic primer”
gcaggtccagttccccaatcgcc

SEQ ID NO: 62 - DNA, 25 bp, Artificial Sequence
  source: /note=“Description of Artificial Sequence Synthetic primer”
cccagacagtttcccaggttacagc

SEQ ID NO: 63 - DNA, 24 bp, Artificial Sequence
  source: /note=“Description of Artificial Sequence Synthetic primer”
tggccaagaagaccattcccgaca

SEQ ID NO: 64 - DNA, 20 bp, Artificial Sequence
  source: /note=“Description of Artificial Sequence Synthetic primer”
cgagccgacactgcgaggct

SEQ ID NO: 65 - DNA, 21 bp, Artificial Sequence
  source: /note=“Description of Artificial Sequence Synthetic primer”
aagtgccaggcggccctgttt

SEQ ID NO: 66 - DNA, 25 bp, Artificial Sequence
  source: /note=“Description of Artificial Sequence Synthetic primer”
aatatgagtgggcaccaccctgtga

SEQ ID NO: 67 - DNA, 25 bp, Artificial Sequence
  source: /note=“Description of Artificial Sequence Synthetic primer”
acacaaccatcatgttgcccaggag

SEQ ID NO: 68 - DNA, 22 bp, Artificial Sequence
  source: /note=“Description of Artificial Sequence Synthetic oligonucleotide”
tagtgcagaaatgagtcagaga

SEQ ID NO: 69 - DNA, 23 bp, Artificial Sequence
  source: /note=“Description of Artificial Sequence Synthetic oligonucleotide”
gaggtttcgggatgactaatact

SEQ ID NO: 70 - DNA, 15 bp, Artificial Sequence
  source: /note=“Description of Artificial Sequence Synthetic oligonucleotide”
tttcggtatgagtca

SEQ ID NO: 71 - DNA, 15 bp, Artificial Sequence
  source: /note=“Description of Artificial Sequence Synthetic oligonucleotide”
tttcagtatgagtca

SEQ ID NO: 72 - DNA, 15 bp, Artificial Sequence
  source: /note=“Description of Artificial Sequence Synthetic oligonucleotide”
tttcagtatgagtca

SEQ ID NO: 73 - DNA, 15 bp, Artificial Sequence
  source: /note=“Description of Artificial Sequence Synthetic oligonucleotide”
tttcggaatgagtca

SEQ ID NO: 74 - DNA, 15 bp, Artificial Sequence
  source: /note=“Description of Artificial Sequence Synthetic oligonucleotide”
tttcgagatgactca

SEQ ID NO: 75 - DNA, 15 bp, Artificial Sequence
  source: /note=“Description of Artificial Sequence Synthetic oligonucleotide”
tttcaaaatgagtca

SEQ ID NO: 76 - DNA, 15 bp, Artificial Sequence
  source: /note=“Description of Artificial Sequence Synthetic oligonucleotide”
tttcattttgactca

SEQ ID NO: 77 - DNA, 15 bp, Artificial Sequence
  source: /note=“Description of Artificial Sequence Synthetic oligonucleotide”
tttctttatgagtca

SEQ ID NO: 78 - DNA, 15 bp, Artificial Sequence
  source: /note=“Description of Artificial Sequence Synthetic oligonucleotide”
tttcagtctgactca

SEQ ID NO: 79 - DNA, 15 bp, Artificial Sequence
  source: /note=“Description of Artificial Sequence Synthetic oligonucleotide”
tttcagtctgactca

SEQ ID NO: 80 - DNA, 15 bp, Artificial Sequence
  source: /note=“Description of Artificial Sequence Synthetic oligonucleotide”
tttctaaatgactca

SEQ ID NO: 81 - DNA, 15 bp, Artificial Sequence
  source: /note=“Description of Artificial Sequence Synthetic oligonucleotide”
tttctaaatgactca

SEQ ID NO: 82 - DNA, 15 bp, Artificial Sequence
  source: /note=“Description of Artificial Sequence Synthetic oligonucleotide”
tttcgctttgactca

SEQ ID NO: 83 - DNA, 15 bp, Artificial Sequence
  source: /note=“Description of Artificial Sequence Synthetic oligonucleotide”
tttcattctgagtca

SEQ ID NO: 84 - DNA, 15 bp, Artificial Sequence
  source: /note=“Description of Artificial Sequence Synthetic oligonucleotide”
tttcattctgagtca

SEQ ID NO: 85 - DNA, 15 bp, Artificial Sequence
  source: /note=“Description of Artificial Sequence Synthetic oligonucleotide”
tttccaaatgactca

SEQ ID NO: 86 - DNA, 15 bp, Artificial Sequence
  source: /note=“Description of Artificial Sequence Synthetic oligonucleotide”
tttcagtttgagtca

SEQ ID NO: 87 - DNA, 15 bp, Artificial Sequence
  source: /note=“Description of Artificial Sequence Synthetic oligonucleotide”
tttccgtatgagtca

SEQ ID NO: 88 - DNA, 15 bp, Artificial Sequence
  source: /note=“Description of Artificial Sequence Synthetic oligonucleotide”
tttcatattgactca

SEQ ID NO: 89 - DNA, 15 bp, Artificial Sequence
  source: /note=“Description of Artificial Sequence Synthetic oligonucleotide”
tttctgaatgactca

SEQ ID NO: 90 - DNA, 15 bp, Artificial Sequence
  source: /note=“Description of Artificial Sequence Synthetic oligonucleotide”
tttctttttgactca

SEQ ID NO: 91 - DNA, 15 bp, Artificial Sequence
  source: /note=“Description of Artificial Sequence Synthetic oligonucleotide”
tttctttctgactca

SEQ ID NO: 92 - DNA, 15 bp, Artificial Sequence
  source: /note=“Description of Artificial Sequence Synthetic oligonucleotide”
tttctttctgactca

SEQ ID NO: 93 - DNA, 15 bp, Artificial Sequence
  source: /note=“Description of Artificial Sequence Synthetic oligonucleotide”
tttcactctgactca

SEQ ID NO: 94 - DNA, 15 bp, Artificial Sequence
  source: /note=“Description of Artificial Sequence Synthetic oligonucleotide”
tttctttttgagtca

SEQ ID NO: 95 - DNA, 15 bp, Artificial Sequence
  source: /note=“Description of Artificial Sequence Synthetic oligonucleotide”
tttccttttgagtca

SEQ ID NO: 96 - DNA, 15 bp, Artificial Sequence
  source: /note=“Description of Artificial Sequence Synthetic oligonucleotide”
tttcagattgagtca

SEQ ID NO: 97 - DNA, 15 bp, Artificial Sequence
  source: /note=“Description of Artificial Sequence Synthetic oligonucleotide”
tttcgttgtgagtca

SEQ ID NO: 98 - DNA, 15 bp, Artificial Sequence
  source: /note=“Description of Artificial Sequence Synthetic oligonucleotide”
tttctgtctgagtca

SEQ ID NO: 99 - DNA, 15 bp, Artificial Sequence
  source: /note=“Description of Artificial Sequence Synthetic oligonucleotide”
tttcattgtgactca

SEQ ID NO: 100 - DNA, 15 bp, Artificial Sequence
  source: /note=“Description of Artificial Sequence Synthetic oligonucleotide”
tttcaagttgactca

SEQ ID NO: 101 - DNA, 15 bp, Artificial Sequence
  source: /note=“Description of Artificial Sequence Synthetic oligonucleotide”
tttctctctgactca

SEQ ID NO: 102 - DNA, 15 bp, Artificial Sequence
  source: /note=“Description of Artificial Sequence Synthetic oligonucleotide”
tttcacattgactca

SEQ ID NO: 103 - DNA, 15 bp, Artificial Sequence
  source: /note=“Description of Artificial Sequence Synthetic oligonucleotide”
tttctctttgactca

SEQ ID NO: 104 - DNA, 15 bp, Artificial Sequence
  source: /note=“Description of Artificial Sequence Synthetic oligonucleotide”
tttccctttgactca

SEQ ID NO: 105 - DNA, 15 bp, Artificial Sequence
  source: /note=“Description of Artificial Sequence Synthetic oligonucleotide”
tttcaaagtgactca

SEQ ID NO: 106 - DNA, 15 bp, Artificial Sequence
  source: /note=“Description of Artificial Sequence Synthetic oligonucleotide”
tttctatgtgactca

SEQ ID NO: 107 - DNA, 15 bp, Artificial Sequence
  source: /note=“Description of Artificial Sequence Synthetic oligonucleotide”
tttcttgttgagtca

SEQ ID NO: 108 - DNA, 15 bp, Artificial Sequence
  source: /note=“Description of Artificial Sequence Synthetic oligonucleotide”
tttcggggtgactca

SEQ ID NO: 109 - DNA, 15 bp, Artificial Sequence
  source: /note=“Description of Artificial Sequence Synthetic oligonucleotide”
tttctggttgagtca

SEQ ID NO: 110 - DNA, 15 bp, Artificial Sequence
  source: /note=“Description of Artificial Sequence Synthetic oligonucleotide”
tttcatcctgactca

SEQ ID NO: 111 - DNA, 15 bp, Artificial Sequence
  source: /note=“Description of Artificial Sequence Synthetic oligonucleotide”
tttccaaatgactca

SEQ ID NO: 112 - DNA, 15 bp, Artificial Sequence
  source: /note=“Description of Artificial Sequence Synthetic oligonucleotide”
tttcagactgactca

SEQ ID NO: 113 - DNA, 15 bp, Artificial Sequence
  source: /note=“Description of Artificial Sequence Synthetic oligonucleotide”
tttcgcagtgagtca

SEQ ID NO: 114 - DNA, 15 bp, Artificial Sequence
  source: /note=“Description of Artificial Sequence Synthetic oligonucleotide”
tcccgaaatgactca

SEQ ID NO: 115 - DNA, 15 bp, Artificial Sequence
  source: /note=“Description of Artificial Sequence Synthetic oligonucleotide”
ttccgaaatgagtca

SEQ ID NO: 116 - DNA, 15 bp, Artificial Sequence
  source: /note=“Description of Artificial Sequence Synthetic oligonucleotide”
tctggaaatgactca

SEQ ID NO: 117 - DNA, 15 bp, Artificial Sequence
  source: /note=“Description of Artificial Sequence Synthetic oligonucleotide”
tcacgaaatgactca

SEQ ID NO: 118 - DNA, 15 bp, Artificial Sequence
  source: /note=“Description of Artificial Sequence Synthetic oligonucleotide”
tcctgaaatgagtca

SEQ ID NO: 119 - DNA, 15 bp, Artificial Sequence
  source: /note=“Description of Artificial Sequence Synthetic oligonucleotide”
tcctgaaatgagtca

SEQ ID NO: 120 - DNA, 15 bp, Artificial Sequence
  source: /note=“Description of Artificial Sequence Synthetic oligonucleotide”
tccagaaatgactca

SEQ ID NO: 121 - DNA, 15 bp, Artificial Sequence
  source: /note=“Description of Artificial Sequence Synthetic oligonucleotide”
tatcgaaatgactca

SEQ ID NO: 122 - DNA, 15 bp, Artificial Sequence
  source: /note=“Description of Artificial Sequence Synthetic oligonucleotide”
tccagaaatgagtca

SEQ ID NO: 123 - DNA, 15 bp, Artificial Sequence
  source: /note=“Description of Artificial Sequence Synthetic oligonucleotide”
tcatgaaatgagtca

SEQ ID NO: 124 - DNA, 15 bp, Artificial Sequence
  source: /note=“Description of Artificial Sequence Synthetic oligonucleotide”
ttatgaaatgactca

SEQ ID NO: 125 - DNA, 15 bp, Artificial Sequence
  source: /note=“Description of Artificial Sequence Synthetic oligonucleotide”
tcaggaaatgagtca

SEQ ID NO: 126 - DNA, 15 bp, Artificial Sequence
  source: /note=“Description of Artificial Sequence Synthetic oligonucleotide”
ctcagaaatgactca

SEQ ID NO: 127 - DNA, 15 bp, Artificial Sequence
  source: /note=“Description of Artificial Sequence Synthetic oligonucleotide”
caatgaaatgagtca

SEQ ID NO: 128 - DNA, 15 bp, Artificial Sequence
  source: /note=“Description of Artificial Sequence Synthetic oligonucleotide”
tactgaaatgactca

SEQ ID NO: 129 - DNA, 15 bp, Artificial Sequence
  source: /note=“Description of Artificial Sequence Synthetic oligonucleotide”
tcgggaaatgactca

SEQ ID NO: 130 - DNA, 15 bp, Artificial Sequence
  source: /note=“Description of Artificial Sequence Synthetic oligonucleotide”
ttgtgaaatgagtca

SEQ ID NO: 131 - DNA, 15 bp, Artificial Sequence
  source: /note=“Description of Artificial Sequence Synthetic oligonucleotide”
ccccgaaatgagtca

SEQ ID NO: 132 - DNA, 15 bp, Artificial Sequence
  source: /note=“Description of Artificial Sequence Synthetic oligonucleotide”
tatggaaatgagtca

SEQ ID NO: 133 - DNA, 15 bp, Artificial Sequence
  source: /note=“Description of Artificial Sequence Synthetic oligonucleotide”
tgcagaaatgactca

SEQ ID NO: 134 - DNA, 15 bp, Artificial Sequence
  source: /note=“Description of Artificial Sequence Synthetic oligonucleotide”
tgcagaaatgagtca

SEQ ID NO: 135 - DNA, 15 bp, Artificial Sequence
  source: /note=“Description of Artificial Sequence Synthetic oligonucleotide”
tgcagaaatgagtca

SEQ ID NO: 136 - DNA, 15 bp, Artificial Sequence
  source: /note=“Description of Artificial Sequence Synthetic oligonucleotide”
gtttgaaatgagtca

SEQ ID NO: 137 - DNA, 15 bp, Artificial Sequence
  source: /note=“Description of Artificial Sequence Synthetic oligonucleotide”
tggtgaaatgactca

SEQ ID NO: 138 - DNA, 15 bp, Artificial Sequence
  source: /note=“Description of Artificial Sequence Synthetic oligonucleotide”
gctagaaatgagtca

SEQ ID NO: 139 - DNA, 15 bp, Artificial Sequence
  source: /note=“Description of Artificial Sequence Synthetic oligonucleotide”
tggtgaaatgagtca

SEQ ID NO: 140 - DNA, 15 bp, Artificial Sequence
  source: /note=“Description of Artificial Sequence Synthetic oligonucleotide”
ctcagaaatgactca

SEQ ID NO: 141 - DNA, 15 bp, Artificial Sequence
  source: /note=“Description of Artificial Sequence Synthetic oligonucleotide”
cccagaaatgagtca

SEQ ID NO: 142 - DNA, 15 bp, Artificial Sequence
  source: /note=“Description of Artificial Sequence Synthetic oligonucleotide”
attggaaatgactca

SEQ ID NO: 143 - DNA, 15 bp, Artificial Sequence
  source: /note=“Description of Artificial Sequence Synthetic oligonucleotide”
agcagaaatgactca

SEQ ID NO: 144 - DNA, 15 bp, Artificial Sequence
  source: /note=“Description of Artificial Sequence Synthetic oligonucleotide”
tacagaaatgactca

SEQ ID NO: 145 - DNA, 15 bp, Artificial Sequence
  source: /note=“Description of Artificial Sequence Synthetic oligonucleotide”
taacgaaatgagtca

SEQ ID NO: 146 - DNA, 15 bp, Artificial Sequence
  source: /note=“Description of Artificial Sequence Synthetic oligonucleotide”
ggaagaaatgactca

SEQ ID NO: 147 - DNA, 15 bp, Artificial Sequence
  source: /note=“Description of Artificial Sequence Synthetic oligonucleotide”
actggaaatgagtca

SEQ ID NO: 148 - DNA, 15 bp, Artificial Sequence
  source: /note=“Description of Artificial Sequence Synthetic oligonucleotide”
ttcagaaatgactca

SEQ ID NO: 149 - DNA, 15 bp, Artificial Sequence
  source: /note=“Description of Artificial Sequence Synthetic oligonucleotide”
cgtagaaatgactca

SEQ ID NO: 150 - DNA, 15 bp, Artificial Sequence
  source: /note=“Description of Artificial Sequence Synthetic oligonucleotide”
tgaggaaatgactca

SEQ ID NO: 151 - DNA, 15 bp, Artificial Sequence
  source: /note=“Description of Artificial Sequence Synthetic oligonucleotide”
ctgagaaatgagtca

SEQ ID NO: 152 - DNA, 15 bp, Artificial Sequence
  source: /note=“Description of Artificial Sequence Synthetic oligonucleotide”
ggaggaaatgactca

SEQ ID NO: 153 - DNA, 15 bp, Artificial Sequence
  source: /note=“Description of Artificial Sequence Synthetic oligonucleotide”
agcagaaatgactca

SEQ ID NO: 154 - DNA, 15 bp, Artificial Sequence
  source: /note=“Description of Artificial Sequence Synthetic oligonucleotide”
cagagaaatgactca

SEQ ID NO: 155 - DNA, 15 bp, Artificial Sequence
  source: /note=“Description of Artificial Sequence Synthetic oligonucleotide”
agcagaaatgactca

SEQ ID NO: 156 - DNA, 15 bp, Artificial Sequence
  source: /note=“Description of Artificial Sequence Synthetic oligonucleotide”
tagagaaatgagtca

SEQ ID NO: 157 - DNA, 15 bp, Artificial Sequence
  source: /note=“Description of Artificial Sequence Synthetic oligonucleotide”
gaaagaaatgactca

SEQ ID NO: 158 - DNA, 15 bp, Artificial Sequence
  source: /note=“Description of Artificial Sequence Synthetic oligonucleotide”
agaggaaatgagtca

SEQ ID NO: 159 - DNA, 15 bp, Artificial Sequence
  source: /note=“Description of Artificial Sequence Synthetic oligonucleotide”
agcagaaatgactca

SEQ ID NO: 160 - DNA, 15 bp, Artificial Sequence
  source: /note=“Description of Artificial Sequence Synthetic oligonucleotide”
agcagaaatgactca

SEQ ID NO: 161 - DNA, 15 bp, Artificial Sequence
  source: /note=“Description of Artificial Sequence Synthetic oligonucleotide”
gaaggaaatgactca

SEQ ID NO: 162 - DNA, 31 bp, Artificial Sequence; identical to SEQ ID NO: 21
  source: /note=“Description of Artificial Sequence Synthetic probe”

SEQ ID NO: 163 - DNA, 35 bp, Artificial Sequence
  source: /note=“Description of Artificial Sequence Synthetic probe”
ttgccttagaggtttcgggatgactaatactgtac

SEQ ID NO: 164 - DNA, 4683 bp, Mus musculus
gggaccctttgctgccctcagctaagagtgcgggtgagcgcacaagcccaggaggaggtccgcacgcgtcatgaacttggagacgggcagccggggctcagagttcggcatgagcgcagtgagctgcggcaatgggaaactccgacagtggttgatcgaccagatcgacagcggcaagtaccccgggctggtgtgggagaacgaggagaagagcgtcttccgcatcccgtggaaacacgcgggcaagcaggactacaatcgtgaggaggacgctgccctcttcaaggcttgggcattgtttaaaggcaagttccgagaagggatcgacaagccagatcctcctacttggaagacaagattacgatgtgctctgaacaagagcaatgactttgaggaattggtcgagaggagccagctggatatctctgacccatacaaggtgtacaggattgttccagagggagccaaaaaaggagcaaagcagctcactttggatgacacacagatggccatgggccacccctaccccatgacagcaccttatggctctctgccagcccagcaggttcataactacatgatgccaccccatgacaggagctggagggattatgcccctgaccagtcacacccagaaatcccatatcaatgtcctgtgacgtttggcccacgaggccaccactggcaaggcccatcttgtgaaaatggttgccaggtgacaggaaccttttatgcttgtgccccacctgagtcccaggctcctggaatccccattgagccaagcataaggtctgctgaagccttggcgctctcagactgccggctgcatatctgcctgtattaccgggacatcctcgtgaaagagctgaccacgacgagccctgaaggctgccggatctcccacggacacacctatgatgttagcaacctggaccaggtcctgtttccctacccggacgacaatggacagaggaagaacattgagaagttgctgagccacctggagaggggactggtcctctggatggctccagatgggctttatgccaaaagactctgccagagtaggatctactgggatgggcccctggcactgtgcagcgatcggcccaacaagctagaaagagaccagacttgcaagctctttgacacacagcagtttctatcagagctgcaagtgtttgctcaccatggccggccagcaccgagattccaggtgactctgtgctttggtgaggagtttccagaccctcagagacagaggaagctcatcacagctcatgtggaacctctgctagccagacaactgtattactttgctcaacaaaacactggacatttcctgaggggctacgagttacctgaacacgttaccactccagattaccaccgctccctccgtcattcttccatccaagagtgagaagaaatactctgacagggcagccggctgctgccctttctctttggaagagctaagaagtgagtgggtttccacttgaagacaacaacagggctttgtgaggaaaaacagctgtatctgctcaacagaggagcttcccccagaagagtgcctgtcatccaggtcttgacaagtgccaggacttgggtgactgtgccctggcttataactgtgaaacttgatcagtgttgtgtttacatgtacttgaatgctggctttagcctggtatagatggacttttgcttgaagactgaaaacctgtgccagcatgaatccctgacaagagaagacatacgtattattggtccatttctcagggaagtaaagtctagactagagactacgctgtccactcacagagaaacgcattcctggatagaggagctgtaggttgcccagatcatgtccactgagtgaagggacttggttcttaggtccagtctaggctatcatgtcctgatcctgcactctctctgagtagaccctacatcctgtttgctccctcttcttgcccttccctaaaagccagctggtggactcttgttgccacatacttgagccaaaaaaccatcagggacatataagatgagacatgtttgcatctcctctgatctaacaacggctacaaagttttttgttgttgttgtctcccctttctgttttgcctctcattctttagaattgttgcttttttgtaaagaaaggaagagagagagaccgaatatactagagatagatttttaaaattctgtgccatttccccttctatttgttttgttggccatcagtggtccccatggcaacactggaagggcggagcctttgagcaggttgggaactgtcccggacaccagtggttcagtgtacagctgtagactcagtgagctgtttctgcttattttgtatattagatgcttccttgtgccaataatagtttgacagagtctcaaagctaccgggcttcttttccaacaacacattctttcacagaatcccaaagagtctcacttgccttcacaaccgtctgatcctttattgttactgttttgtttaggtggctttgccatgctctgggatttttctcctaccacaacatcattcctgactctttcgggctcagaaagagttaagcgctcttgcacacttggcacttacttctttttaattttttttttaacatgccactcttaatatttcttcttaagtctttcagcagcatacattcaataagcaatggaccattgggtgtctcatttcattgttttatttttaaaaaggaacctttgacaaataatacacattacagaaaatggaagcatttttcatagagaaggtaagaactgaaatgcattttatgttcgaatcctccttgctagtattagccccaagagttcagtctgtcctttaagcatgttttagttttcaatacatatatatgaagttgccctttcttagagagagagagagagatatgtgatgacactcttatgtacccaactatacagatttacctcatcttttgagtggcatgtggacttccactgctatgtgcctaggtgcagtttagacatcttcttagctacccatcccagcagcccactgagtctcgtgaactaaagtcacttatacatcttatagatgcaaaacattctggggtatattatcccaagggaagatgaaggcaaaatcgggaagcactcttcctgaggccccaagccacagctgtcctcctggagtatcttaaccatatctggcacttcaccaaggtcccttgcctggtcctgtatgaccatgactcttcagtccataagcagatgctttgtctcaattgccatgtgaagtcttttggtttttggtttttttccttaatgttaagctgttttaacagtgaagctgaattttctggaaaatgctgtatggttcgagcccccagttcatgtttcactttacagccatgcatgctgatgccttccatcttgacaaatggaatgatgggtaaacactcctgacacaaagaggaacaccttcctcgtcccttgctgaaacaaagtgcaatgcacacagtagaatttagtttgcttgcactggaagatgccgttgaagaggtaggctgaggaacactgtatcctagggagaaaatatgcaagtctcacgggtgctttctgttggcttgtttctggtccttgagcaggcttaagtatctagtttcaaactagaagccccaaagccctcagtcgttgtcctggacagagttggtagaccccacctgtagaaatccagatgcactctgggattggagcacagagaggcttgctctatcgtgagaatgcccagcctgcagaggcccaggtgcggtgcctgagtggctgtatgccagacacaggaacgccagcaggctgagtgtggtgactgatatgtgaggtgcccttgccctgcagacaggctgaatgtgagccccagcagcagaggattctgagagtcagggtggtgtcatgtcctacacaggaagagccttcaatattactttttaaacgaactttcccattgctgatattttgataaaaacccacacaaggtttaattacatgccccctcatccctctccacacagtcttgcccatcccactggtgaattgttgttgttcttgttgttgttttttgttattggtgaaaattgtgaaattgttaaggtctctgtttttgttatttatctccctaaatcacatggctgctgcatacattgcaatgttgccctaccacatgtatgtttttgcaagttgcttcttcactcattgaagaagcctgcaccgtaagtgaaactcacttagtccttgctaattgctgctcactgtcacgtgaacgcagagaaggctgattttcctggacccattttcagtgctttgaggaacatgagtccagagtatattgaagtagaagacagtgtttggcaagccagttcttattagacactgctacaggcttcacaatcttcaaggtggacccacctcatgtggaagggcatcattgcccctctgctcccatgcgctgtgcacccccttcacagtgaaagacctgctggggaccaggactcagagatctctggcttcgagctcatatcttggcttcctgtcatgtctaagcccgacaggaaagtgtggcaccatcctgcctgcggcgttggatggataggccatggattttgctgtaaacttgcttcacaa

SEQ ID NO: 165 - protein, 450 aa, Mus musculus
MNLETGSRGSEFGMSAVSCGNGKLRQWLIDQIDSGKYPGLVWENEEKSVFRIPWKHAGKQDYNREEDAALFKAWALFKGKFREGIDKPDPPTWKTRLRCALNKSNDFEELVERSQLDISDPYKVYRIVPEGAKKGAKQLTLDDTQMAMGHPYPMTAPYGSLPAQQVHNYMMPPHDRSWRDYAPDQSHPEIPYQCPVTFGPRGHHWQGPSCENGCQVTGTFYACAPPESQAPGIPIEPSIRSAEALALSDCRLHICLYYRDILVKELTTTSPEGCRISHGHTYDVSNLDQVLFPYPDDNGQRKNIEKLLSHLERGLVLWMAPDGLYAKRLCQSRIYWDGPLALCSDRPNKLERDQTCKLFDTQQFLSELQVFAHHGRPAPRFQVTLCFGEEFPDPQRQRKLITAHVEPLLARQLYYFAQQNTGHFLRGYELPEHVTTPDYHRSLRHSSIQE

SEQ ID NO: 166 - DNA, 2829 bp, Mus musculus
ccggaggcgcgggcagcgtgggaaccggcggcaggatgtgtgaccggaacggcgggcggcggctgcggcagtggctgatcgaacagatcgacagcagcatgtacccggggctgatctgggaaaatgatgagaagaccatgttccgtatcccctggaagcatgccggcaagcaggattacaatcaggaggtggatgcttccatcttcaaggcctgggcagtttttaaagggaagtttaaagagggagacaaagctgaaccagccacgtggaagacgaggttacgctgtgctctgaacaagagcccagattttgaagaagtgactgaccggtcccagctggacatttctgagccatataaagtttaccgaattgtccccgaggaagaacaaaaatgcaagctgggcgtggcacctgcaggctgcatgagcgaagttcctgagatggagtgtggccgctcagagattgaggagctgatcaaggaaccttctgtggatgagtacatgggtatgaccaagaggagcccatccccaccagaggcctgcaggagccagatcctccctgactggtgggtccagcagcccagtgcaggcctgccactggtgaccggatatgccgcctatgacacacaccattcagctttctcccagatggtcatcagcttctactacgggggcaagctggtgggccaggccaccaccacctgccttgaaggctgccgtctctccctgagccagccggggctgcctaagttgtatgggccggatggcctggaacccgtgtgctttccgacggccgacaccatccccagtgagcggcagaggcaggtgacccggaagctgtttgggcacctggaacgtggcgtgctactgcacagcaaccgcaagggcgtgttcgtgaagcggctgtgccagggccgcgtgttctgcagcggcaacgcggtggtgtgcaagggcaggcccaacaagctggagcgggacgaggtggtgcaggtctttgacaccaaccagttcatccgagagctgcagcaattctacgccacccagagccgcctacctgacagcagggtggtcctgtgcttcggggaggagtttccggacactgtgcccttgcgctccaaactcattctggtgcaggtagagcagctgtatgccaggcagctggtggaggaagcgggcaagagctgcggtgctggctccctgatgccagccctggaggagccccagccggaccaggctttccgcatgtttccggatatctgtacctcacaccagagacccttttttagagaaaatcaacagatcaccgtctaagcctcagtccgggcaccccacctcgcctgagctcaagcttcaagagtctgtgactaagagaattccgaaaggatgtggagccctctgactggggtgggcgggtgtcctccaaggggcctccggaagcccacagagggatgcgctcctgctcaggcaggtgtcagaagcttgcaggggctgtggccgcaacctgtgattaaagcattcctttcctgcgtttcccccttcaccactaatggctggcctttctgtgtgctgaggtctttcgacagttcaaatcatctggtggcagcagactcgcctttgcccttctgcggccgagggcggagatttatgactttctctgcttggttggagaagaagaatctttactattcagcttcttttctttttggccagaactctgaaaaaaaaaaaaactctttttaagacaatatttgtattctcacaggctcagctgtcaatcacttgagaccttccctgtaaagtggggcagattttaaatatgggtgtagatactgcttgcagccttcgcaggaattttggttgtggttcattgattcacacagactctgtgtcagctgacagggctgtgtggggcatcaaaggaggaccaggcactgtggagaagacccattcactggcatctcacccttccttgtccagctccatacccagtcctaagacccagtgaaaagccacgtccaaactgtgctctgggctcatcagtgcccacccacgtaccagggaaaggcacacaccctacccagtgggcacagagcggaatgtccccctaccgcaccatttgcgcccccaatctggctgtccaacctagtttgtaagtaatctaaatcagtgactatagccccgcctaagggacacttcccggaggagggagccgctgaaaaggagttagtttgagggtcagtacacaacaggggcagaaagccaagcagatgtgggggcagggagagtcatcatctgcttttgtctgagagaaggagagcttctccgtttgttcaactttgtaacaagctgggttacatgctccacgcagctagagaagcctaggtgctctgcattccctggggaactgcaggaaagccttacctgctgactgttgctctggggaaaagcctgagggtccagagcagctacaagctacaggccataccttacaacctgaaaagctaaggaccacggtgaccttcccggctactgtgtgaaggtgctgggtggggcctgctcaacagacagggtcgacagagtgtgtgatacatgcaaacagaatccttggagtgtgtgatacatgcaaacagaatcctgggcccctgcttctccccctcagtcaaagcaggagtgtcccttccgaagccaggacaacctgttcacaaggccccttgtcacatgtcaccttccacctgcctcaaggagtgctagtgtccaaatatttatttttgtattctcttaagaagtattgatttcatcctttattaaaaaaagttgctctttcacaaaa

SEQ ID NO: 167 - protein, 424 aa, Mus musculus
MCDRNGGRRLRQWLIEQIDSSMYPGLIWENDEKTMFRIPWKHAGKQDYNQEVDASIFKAWAVFKGKFKEGDKAEPATWKTRLRCALNKSPDFEEVTDRSQLDISEPYKVYRIVPEEEQKCKLGVAPAGCMSEVPEMECGRSEIEELIKEPSVDEYMGMTKRSPSPPEACRSQILPDWWVQQPSAGLPLVTGYAAYDTHHSAFSQMVISFYYGGKLVGQATTTCLEGCRLSLSQPGLPKLYGPDGLEPVCFPTADTIPSERQRQVTRKLFGHLERGVLLHSNRKGVFVKRLCQGRVFCSGNAVVCKGRPNKLERDEVVQVFDTNQFIRELQQFYATQSRLPDSRVVLCFGEEFPDTVPLRSKLILVQVEQLYARQLVEEAGKSCGAGSLMPALEEPQPDQAFRMFPDICTSHQRPFFRENQQITV

SEQ ID NO: 168 - DNA, 904 bp, Mus musculus
gcagtccctctgcacccgagagagaggaggacgcaggggtctgtcagaggttgctgttgggcaagcaggggaggtacctgtggaaggtggtgtgctggtggccccctagcagtcaagaaggggagccagctagtgagaagatcgcccagaggcatctgggacggtgtgggagagcccggaagattagaaccatgcctcacagctccgacagcagtgactccagcttcagccgctctcctccccctggcaaacaggactcatctgatgatgtgaggaaagttcagaggagagagaagaatcgcatcgctgcccagaagagccgacagagacagacacagaaagccgacacccttcacctggagagtgaggacctggagaaacagaacgcagctctccgcaaagagatcaaacagctcaccgaggagctcaagtacttcacatcagtgctgagcagccacgagcccctgtgctccgtgctggccagtggcaccccctcgccccccgaggtggtatacagtgcccatgccttccaccagcctcacatcagctcgccacgcttccagccctgaccttctggacaagaagggcgatgctactcccgtgatcccttggaggggcatgtaaactgaggccgggctgccctcatacctctacccagaggcccagtggcagaggcctggacaagtattgaacacaagaactgtagtggtcagagggacttaaggcctcccagggaagtatagtcaatgtactggactctcccagggaagtcgagccaatgtactggacccaaaaaatgacaagtcaaccctggactgtcatgaatgatgcccaaaatacacagcacagagggaggagggcagggggtggatagttttctaaataaatattttctaaaaaacca

SEQ ID NO: 169 - protein, 125 aa, Mus musculus
MPHSSDSSDSSFSRSPPPGKQDSSDDVRKVQRREKNRIAAQKSRQRQTQKADTLHLESEDLEKQNAALRKEIKQLTEELKYFTSVLSSHEPLCSVLASGTPSPPEVVYSAHAFHQPHISSPRFQP

SEQ ID NO: 170 - DNA, 1840 bp, Mus musculus
tataaaagcttggggctggggccgagcactggggactttgagggtggccaggccagcgtaggatcctgctgggagcggggaactgagggaagcgacgccgagaaagcaggcgcaccacggagggagagaaaatctccggaagcccagcagcgcctttacgcacagctgccaactggccgctgctgaccgtctccagctcccgaggacgcgcgaccggacaccgggtcctgccacagccgaggacagctcgccgctcgccgcagcgaggcccggggcggcccttcagggggacctttcccagatcgcccaggccgcccggatgtgcacgaaaatggaacagcctttctatcacgacgactcttacgcagcggcgggatacggtcggagccctggcagcctgtctctacacgactacaaactcctgaaacccaccttggcgctcaacctggcggatccctatcggggtctcaagggtcctggggcgcggggtccaggcccggagggcagtggggcaggcagctacttttcgggtcagggatcagacacaggcgcatctctgaagctagcctccacggaactggagcgcttgatcgtccccaacagcaacggcgtgatcacgacgacgcccacgcctccgggacagtacttttacccccgtgggggtggcagcggtggaggtacagggggcggcgtcaccgaggagcaggagggctttgcggacggttttgtcaaagccctggacgacctgcacaagatgaaccacgtgacgccccccaacgtgtccctgggcgccagcgggggtccccaggccggcccagggggcgtctatgctggtccggagccgcctcccgtctacaccaacctcagcagttactccccagcctctgcaccctctggaggctccgggaccgccgtcgggactgggagctcatacccgacggccaccatcagctacctcccacatgcaccaccctttgcgggcggccacccggcacagctgggcttgagtcgcggcgcttccgcctttaaagaggaaccgcagaccgtaccggaggcacgcagccgcgacgccacgccgcctgtgtcccccatcaacatggaagaccaggagcgcatcaaagtggagcgaaagcggctgcggaacaggctggcggccaccaagtgccggaagcggaagctggagcgcatcgcgcgcctggaggacaaggtgaagacactcaaggctgagaacgcggggctgtcgagtgctgccggtctcctacgggagcaagtggcgcagctcaagcagaaggtcatgacccatgtcagcaacggctgccagttgctgctaggggtcaagggacacgccttctgagagcctcccttgccccatacggacacccccagccttgaaggctgggcgcctgccccccactggggtgaggggggcaggcgatgggcacccgccaaaaggcctggggcgcagctcacacactggactccggcccgcccgcctgcgcccagtccttccacctcgaggtttacatggcccccttccagcgtattttgtatgttttttttttctgcaaagagactgaattcatattgaatataatatatttgtgtatttaacagggaggggagaagggggctgtcgcggcggagctggccccgccgcttggtactcagcctgcggggatactagggagggacctccgccccctgccctccccctctgcatagtactgtggagaagaaacacgcacttcgtgtctaaagtctattttaagatgtgtttgtgtgtgtgtgtttgactttttattgaatctatttaagtaaaaaaaaaattgtctttattaat

SEQ ID NO: 171 - protein, 344 aa, Mus musculus
MCTKMEQPFYHDDSYAAAGYGRSPGSLSLHDYKLLKPTLALNLADPYRGLKGPGARGPGPEGSGAGSYFSGQGSDTGASLKLASTELERLIVPNSNGVITTTPTPPGQYFYPRGGGSGGGTGGGVTEEQEGFADGFVKALDDLHKMNHVTPPNVSLGASGGPQAGPGGVYAGPEPPPVYTNLSSYSPASAPSGGSGTAVGTGSSYPTATISYLPHAPPFAGGHPAQLGLSRGASAFKEEPQTVPEARSRDATPPVSPINMEDQERIKVERKRLRNRLAATKCRKRKLERIARLEDKVKTLKAENAGLSSAAGLLREQVAQLKQKVMTHVSNGCQLLLGVKGHAF

SEQ ID NO: 172 - DNA, 3187 bp, Mus musculus
gttgagctcaggctggataaggactcagagttgcactgagtgtggcagagacagcctggcaggagagcgctcaggcagacagacagacagacggacggacttggccaacccggtcggccgcggactccggactgttcatccgtttgtcttcattttctcaccaactgcttggatccagcgcccgcggctcctgcaccggtattttggggagcatttggagagtcccttctcccgccttccacggagaagaagctcacaagtccgggcgctgctgacagcatcgagagcggctcccgaccgcgcgaggaaataggcgagcggctaccggccagcaactttcctgacccagaggaccggtaacaagtggccgggagcgaacttttgcaaatctcttctgcgccttaaggctgccaccgagactgtaaagaaaagggagaagaggaacctatactcataccagttcgcacaggcggctgaagttgggcgagcgctagccgcggctgcctagcgtccccctccccctcacagcggaggaggggacagttgtcggaggccgggcggcagagcccgatcgcgggcttccaccgagaattccgtgacgactggtcagcaccgccggagagccgctgttgctgggactggtctgcgggctccaaggaaccgctgctccccgagagcgctccgtgagtgaccgcgacttttcaaagctcggcatcgcgcgggagcctaccaacgtgagtgctagcggagtcttaaccctgcgctccctggagcgaactggggaggagggctcagggggaagcactgccgtctggagcgcacgctcctaaacaaactttgttacagaagcggggacgcgcgggtatccccccgcttcccggcgcgctgttgcggccccgaaacttctgcgcacagcccaggctaaccccgcgtgaagtgacggaccgttctatgactgcaaagatggaaacgaccttctacgacgatgccctcaacgcctcgttcctccagtccgagagcggtgcctacggctacagtaaccctaagatcctaaaacagagcatgaccttgaacctggccgacccggtgggcagtctgaagccgcacctccgcgccaagaactcggaccttctcacgtcgcccgacgtcgggctgctcaagctggcgtcgccggagctggagcgcctgatcatccagtccagcaatgggcacatcaccactacaccgacccccacccagttcttgtgccccaagaacgtgaccgacgagcaggagggcttcgccgagggcttcgtgcgcgccctggctgaactgcatagccagaacacgcttcccagtgtcacctccgcggcacagccggtcagcggggcgggcatggtggctcccgcggtggcctcagtagcaggcgctggcggcggtggtggctacagcgccagcctgcacagtgagcctccggtctacgccaacctcagcaacttcaacccgggtgcgctgagcagcggcggtggggcgccctcctatggcgcggccgggctggcctttccctcgcagccgcagcagcagcagcagccgcctcagccgccgcaccacttgccccaacagatcccggtgcagcacccgcggctgcaagccctgaaggaagagccgcagaccgtgccggagatgccgggagagacgccgcccctgtcccctatcgacatggagtctcaggagcggatcaaggcagagaggaagcgcatgaggaaccgcattgccgcctccaagtgccggaaaaggaagctggagcggatcgctcggctagaggaaaaagtgaaaaccttgaaagcgcaaaactccgagctggcatccacggccaacatgctcagggaacaggtggcacagcttaagcagaaagtcatgaaccacgttaacagtgggtgccaactcatgctaacgcagcagttgcaaacgttttgagaacagactgtcagggctgaggggcaatggaagaaaaaaaataacagagacaaacttgagaacttgactggttgcgacagagaaaaaaaaagtgtccgagtactgaagccaagggtacacaagatggactgggttgcgacctgacggcgcccccagtgtgctggagtgggaaggacgtggcgcgcctggctttggcgtggagccagagagcagcggcctattggccggcagactttgcggacgggctgtgcccgcgcgcgaccagaacgatggacttttcgttaacattgaccaagaactgcatggacctaacattcgatctcattcagtattaaaggggggtgggaggggttacaaactgcaatagagactgtagattgcttctgtagtgctccttaacacaaagcagggagggctgggaaggggggggaggcttgtaagtgccaggctagactgcagatgaactcccctggcctgcctctctcaactgtgtatgtacatatatatttttttttaatttgatgaaagctgattactgtcaataaacagcttcctgcctttgtaagttattccatgtttgtttgtttgggtgtcctgcccagtgtttgtaaataagagatttgaagcattctgagtttaccatttgtaataaagtatataatttttttatgttttgtttctgaaaatttccagaaaggatatttaagaaaatacaataaactattgaaaagtagcccccaacctctttgctgcattatccatagataatgatagctagatgaagtgacagctgagtgcccaatatactagggtgaaagctgtgtcccctgtctgattgtaggaatagataccctgcatgctatcattggctcatactctctcccccggcaacacacaagtccagactgtacaccagaagatggtgtggtgtttcttaaggctggaagaagggctgttgcaaggggagagggtcagcccgctggaaagcagacactttggttgaaagctgtatgaagtggcatgtgctgtgatcatttataatcataggaaagatttagtaattagctgttgattctcaaagcagggacccatggaagtttttaacaaaaggtgtctccttccaactttgaatctgacaactcctagaaaaagatgacctttgcttgtgcatatttataatagcgttcgttatcacaataaatgtattcaaataatggtttttaaaatcttg

SEQ ID NO: 173 - protein, 334 aa, Mus musculus
MTAKMETTFYDDALNASFLQSESGAYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVSGAGMVAPAVASVAGAGGGGGYSASLHSEPPVYANLSNFNPGALSSGGGAPSYGAAGLAFPSQPQQQQQPPQPPHHLPQQIPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

(19) United States
(12) Patent Application Publication
(10) Pub. No.: US 2014/0378537 A1
(43) Pub. Date: Dec. 25, 2014

(54) TREATMENT OF TH17 MEDIATED INFLAMMATORY DISEASES

(71) Applicant: GENENTECH, INC., South San Francisco, CA (US)

(72) Inventors: Elke Glasmacher, Munich (DE); Smita Agrawal, Bangalore (IN); Wenwen Zeng, Beijing (CN); Harinder Singh, Newport, KY (US); Abraham Chang, San Mateo, CA (US)

(21) Appl. No.: 14/343,255

(22) PCT Filed: Sep. 7, 2012
(86) PCT No.: PCT/US2012/054253

§ 371 Date: Sep. 8, 2014

Related U.S. Application Data
(60) Provisional application No. 61/532,861, filed on Sep. 9, 2011.
(60) Provisional application No. 61/687,449, filed on Apr. 24, 2012.
(60) Provisional application No. 61/688,972, filed on May 25, 2012.

(51) Int. Cl. C12N 15/113; C12Q 1/68

(52) U.S. Cl. CPC C12N 15/1136; C12Q 1/6816
USPC 514/44 R; 536/24.1; 435/6.11

(57) ABSTRACT

The present invention provides methods and means to reduce inflammation associated with IRF-4, AP-1 and TH17 mediated diseases. In particular, the invention provides methods and means to treat multiple sclerosis, rheumatoid arthritis, inflammatory bowel disease, and psoriasis and related conditions.

### CROSS-REFERENCE TO RELATED APPLICATIONS

This application claims priority under Section 119(e) and the benefit of U.S. Provisional Application Ser. Nos. 61/532,861 filed Sep. 9, 2011, 61/687,449 filed Apr. 24, 2012, and 61/688,972 filed May 25, 2012, each of which the entire disclosures are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Sep. 7, 2012, is named GNE38PCT.txt and is 74,697 bytes in size.

### BACKGROUND OF THE INVENTION

Th17 cells are a subset of T helper cells that function in host defense by producing the pro-inflammatory cytokines IL-17 and TNFa. They also contribute to autoimmune inflammatory conditions such as experimental autoimmune encephalomyelitis. IRF-4 is a transcription factor that is required for the generation of Th17 and Th2 cells as well as the development and functioning of B cells. IRF-4 is an immune system specific member of the IRF transcription factor family that is required for the differentiation of innate as well as adaptive immune cells. Based on its cloning and initial characterization by three independent groups it was termed PIP (PU.1 interaction partner), ICSAT (ICSBP in adult T-cell leukemia-specific IRF) and LSIRF (lymphoid-specific IRF). IRF-4 is closely related structurally to IRF-8 and the latter is also expressed specifically in cells of the immune system. IRF-4 and IRF-8 perform key biological functions within the immune system in either a redundant or unique manner depending on the cellular context. IRF-4 and IRF-8 regulate B, T, macrophage and dendritic cell differentiation. IRF-4 is specifically required for B cells to undergo class switch recombination and plasma cell differentiation. It also regulates the generation and/or functioning of alternatively activated macrophages as well as various types of helper T cells including Th17, Th2, Tfh and iTregs. The molecular mechanisms by which IRF-4 participates in programming diverse patterns of gene expression in the fore-mentioned immune cells, particularly those of the T-lineage are poorly understood.

IRF-4 and IRF-8 bind with low affinity to IRF sites and are recruited to genes containing Ets-IRF composite elements (EICE) in cells that express Ets factors PU.1 or Spi-B. IRF-4, in particular, binds weakly to DNA but is recruited to its target sequences by highly specific interactions with Ets family members (PU.1 and Spi-B). Although PU.1 and Spi-B serve as partners of IRF-4 in B cells, lack of their expression in Th17 cells suggest the existence of novel partner(s) for IRF-4 in Th17 cells. Unlike other members of the mammalian interferon-regulatory factor family IRF-4 and -8 bind with low affinity to interferon sequence response elements (ISREs). These sequences (GAAANNGAAA) (SEQ ID NO: 1) contain a dimeric GAAA core motif that is specifically contacted by the IRF DNA binding domain. The lower affinity interaction of the DBDs of IRF-4 and -8 with the GAAA motif is due to their structural divergence from other members of the IRF family. Consequently, it appears that IRF-4 and -8 have evolved to interact with other transcription factors so as to facilitate their recruitment to genomic regulatory elements and to regulate distinctive repertoires of target genes in various types of immune cells. The best characterized interaction partners for IRF-4 and -8 are the structurally related Ets family transcription factors, PU.1 and Spi-B. The latter are able to recruit IRF-4 or -8 to composite Ets-IRF motifs (EICE) that are comprised by the canonical sequence GGAANNGAAA (SEQ ID NO: 2). PU.1 and IRF-4 have been shown to cooperatively assemble as a heterodimer on the EICE motif and the atomic structure of such a ternary complex has revealed specific interactions between the Ets and the IRF DNA binding domains (DBDs). IRF-4 has also been shown to interact with NFATc and this complex is implicated in the regulation of the IL-4 gene in Th2 cells. However, the generality of this mode of DNA recruitment and the biochemical mechanism underlying DNA co-binding as well as the sequence motif needed for assembly of such complexes remains to be explored. Recently, ChIPseq analysis of IRF-4 in activated T cells has revealed co-binding with Stat3 to a large set of genes. The molecular mechanism underlying co-binding of IRF-4 and Stat-3, to this set of presumptive regulatory elements, is not defined and appears to be independent of any physical interaction as the genome-wide analysis did not reveal a canonical Stat-3-IRF composite motif. Furthermore IRF-4 and Stat3 were not seen to cooperatively bind to a DNA sequence containing adjacent IRF and Stat3 motifs. Thus in cells that do not express PU.1 or Spi-B, it is uncertain as to how IRF-4 is recruited to its various genomic target sequences.

Th17 cells are a subset of T helper cells that primarily function in clearance of extracellular pathogens, but also play a major role in a variety of experimentally induced autoimmune diseases, such as colitis, encephalomyelitis and psoriasis. Th17 cells secrete the pro-inflammatory and anti-microbial cytokines IL-17A/F and IL-22, respectively. They also express the cytokine IL-21 that feeds back to regulate their generation in conjunction with TGF-β. Th17 cells additionally depend on IL-23 signaling for stabilization of their differentiated state and become responsive to this cytokine by up regulating the receptor for IL-23, which is composed of the IL-23R and IL-12Rβ1 subunits. IRF-4 deficient mice are protected against experimental autoimmune encephalomyelitis (EAE) and colitis, the latter induced by chemical damage to the intestine. IRF-4−/− T cells differentiated under Th17-polarizing conditions, fail to express IL-17A, IL-22, IL-21 and IL-23R. Although IRF-4 has been shown to directly target some of these key Th17 genes, the means by which it is recruited to their regulatory elements remains to be elucidated.

Th17 differentiation depends on a combinatorial set of transcription factors that includes RORγt, STAT3, IRF-4 and BATF. RORγt is a lineage-specific regulator whose forced expression in activated T cells is sufficient to induce the expression of IL-17A/F and IL-22. RORγt levels are decreased in IRF-4−/− and BATF−/− T cells suggesting that these two transcription factors function in part to induce RORγt expression. However, restoring expression of RORγt in IRF-4−/− or BATF−/− T cells results in a partial rescue of the Th17 differentiation program. Therefore, IRF-4 and BATF are likely required for the direct activation of Th17 genes independently of or in concert with RORγt. Consistent with this proposition a BATF/JunB heterodimer has been shown to bind to the IL-17, IL-21 and IL-22 promoters, as well as to an intergenic region within the IL-17A/F locus. It is noteworthy that certain members of the IRF and AP-1 family, for example, IRF-3 and an ATF-2/c-Jun heterodimer have been shown to interact and cooperatively assemble on the β-IFN gene promoter. Such an interaction raised the possibility that IRF-4 may cooperatively bind with BATF/JunB complexes to regulatory sequences in the context of Th17 differentiation.

Using ChIPseq and biochemical analysis, BATF has been identified as major new interaction partner for IRF-4. It is demonstrated herein that IRF-4 targets sequences enriched for AP-1-IRF composite elements (AICE). The majority of these sequences are co-bound by BATF, an AP-1 family member that is also required for Th17 cell differentiation. IRF-4 and a BATF/JunB heterodimer assemble cooperatively in a DNA dependent manner on structurally diverse AICE motifs having distinct and unusual spacing requirements. IRF-4/BATF/JunB complexes are shown to cooperatively assemble on presumptive regulatory elements within the IL-17A/F locus as well as the IL-21, IL-23R and IL-12R/3 genes. Network analysis of IRF-4/BATF co-bound and regulated genes reveals regulatory modules underlying Th17 differentiation. The AICE motif directs the assembly of IRF-4 or -8 with specific AP-1 family members and is also utilized in Th2, B and dendritic cells. The composite AICE motif revealed in this study has broader implications for the molecular functions of IRF-4 not only in T helper cells but also in B-lymphocytes, macrophages and dendritic cells. It is proposed that this genomic regulatory element and its cognate IRFs have evolved to transduce and integrate diverse immuno-modulatory signals.

### SUMMARY OF THE INVENTION

The present invention is based, at least in part, on experimental data demonstrating that 1) IRF4 binds to a large set of target sequences with neighboring AP-1 sites; 2) the AP-1 family member BATF binds to many of the same genomic regions as IRF4; 3) IRF4 and BATF cooperatively bind to a representative set of target sequences containing novel IRF/AP-1 composite motifs; 4) IRF4 and BATF cooperatively induce expression of the linked CTLA-4 and ICOS genes that contain a composite IRF/AP-1 motif; and 5) ectopic expression of IRF-4 and BATF in activated T cells under Th17 cell polarizing conditions increases the frequency of IL17A-producing cells. The present invention is also based, at least in part, on experimental data demonstrating that 1) IRF-4 and a BATF/JunB heterodimer assemble cooperatively on structurally distinct AP-1-IRF composite elements (AICE), and 2) AICE motifs are associated with genes that comprise transcriptional sub-networks underlying Th17 differentiation, and 3) the AICE motif greatly expands the molecular activities of IRF-4 in innate and adaptive cells of the immune system. Accordingly, described herein are novel compositions and methods for modulating IRF4, BATF and Th17 activity and/or mediated inflammatory diseases.

The present invention provides methods and means to reduce autoimmunity associated with IRF-4, BATF and Th17 cells. In particular, the invention provides methods and means to treat multiple sclerosis, rheumatoid arthritis, inflammatory bowel disease, psoriasis and related conditions.

In one aspect, the present invention provides an isolated molecular complex comprising IRF-4 and an AP-1 family member. In some embodiments, the present invention provides an isolated molecular complex consisting of IRF-4 and an AP-1 family member. In certain other embodiments, the present invention provides an isolated molecular complex consisting essentially of IRF-4 and an AP-1 family member. In certain embodiments, the AP-1 family member is BATF. In some embodiments, the isolated molecular complex further comprises an additional AP-1 family member. In one embodiment, wherein the additional AP-1 family member is JunB. In another embodiment, the additional AP-1 family member is c-Jun.

In one aspect, the present invention provides an isolated molecular complex comprising IRF-4 and an AP-1 family heterodimer, the AP-1 family heterodimer comprising BATF and JunB. In some embodiments, the present invention provides an isolated molecular complex consisting of IRF-4 and an AP-1 family heterodimer, the AP-1 family heterodimer comprising BATF and JunB. In another embodiment, the AP-1 family heterodimer comprises BATF and c-Jun.

In some embodiments, the isolated molecular complex of the invention further comprises a DNA sequence comprising an AP-1/IRF composite motif. In one embodiment, the present invention provides an isolated molecular complex consisting of IRF-4, an AP-1 family member or AP-1 family heterodimer, and a DNA sequence comprising an AP-1/IRF composite motif. In another embodiment, the present invention provides an isolated molecular complex consisting essentially of IRF-4, an AP-1 family member or AP-1 family heterodimer, and a DNA sequence comprising an AP-1/IRF composite motif. In certain embodiments, the DNA sequence is derived from genes expressed in T helper cells. In another embodiment, the DNA sequence is derived from a gene targeted by the IRF-4 and AP-1 complex.

In certain embodiments, the IRF/AP-1 composite motif comprises an IRF site and an AP-1 site. In one embodiment, the IRF/AP-1 composite motif consists of an IRF site and an AP-1 site. In another embodiment, the IRF/AP-1 composite motif consists essentially of an IRF site and an AP-1 site. In one embodiment, the AP-1/IRF composite motif comprises a sequence provided in FIG. 1C. In further embodiments, the molecular complex of the invention is capable of binding an AP-1/IRF composite motif comprising an IRF site and an AP-1 site. In yet further embodiments, the molecular complex of the invention is capable of binding an AP-1/IRF composite motif consisting of an IRF site and an AP-1 site. In still further embodiments, the molecular complex of the invention is capable of binding an AP-1/IRF composite motif consisting essentially of an IRF site and an AP-1 site. In one embodiment, the AP-1/IRF composite motif comprises a 4-bp space between the IRF site and the AP-1 site. In one embodiment, the AP-1/IRF composite motif comprises the sequence TTTC(N4)TGA(G/C)T(C/A)A. In another embodiment, the AP-1/IRF composite motif comprises a 3-bp space between the IRF site and the AP-1 site. In still another embodiment, the AP-1/IRF composite motif comprises a 2-bp space between the IRF site and the AP-1 site. In a further embodiment, the AP-1/IRF composite motif comprises a 1-bp space between the IRF site and the AP-1 site. In another embodiment, the AP-1/IRF composite motif comprises no space between the IRF site and the AP-1 site. In one embodiment, the AP-1/IRF composite motif comprises the sequence GAAATGA(G/C)T(C/A)A. In a specific embodiment, the molecular complex of the invention is capable of binding an AP-1/IRF composite motif where the IRF site is TTTC and the AP-1 site is TGA(C/G)TCA.

In certain embodiments, the molecular complex of the invention, upon binding the AP-1/IRF composite motif, is capable of inducing expression of one or more T helper cell genes. In one embodiment, the T helper cell gene is CTLA-4. In another embodiment, the T helper cell gene is ICOS. In a further embodiment, the molecular complex of the invention, upon binding the AP-1/IRF composite motif, is capable of inducing the differentiation of Th17 cells. In another embodiment, the molecular complex of the invention, upon binding the AP-1/IRF composite motif, is capable of increasing the frequency of 1L-17A-producing cells.

In another embodiment, the isolated molecular complex of the invention, upon binding the AP-1/IRF composite motif, is capable of regulating one or more genes selected from the group consisting of genes listed in FIG. 7A, FIG. 7C, FIG. 8A, and FIG. 8B.

In another aspect, the present invention provides a crystalline form of a complex between IRF-4 and an AP-1 family member. In certain embodiments, the AP-1 family member is BATF.

In still another aspect, the present invention provides a crystalline form of a complex between IRF-4, an AP-1 family member, and a DNA sequence comprising an AP-1/IRF composite motif. In one embodiment, the AP-1 family member is BATF.

In one embodiment, the present invention provides a method for identifying an agent characterized by the ability to inhibit IRF-4/AP-1 family member interaction comprising a) incubating a reaction mixture comprising a candidate agent to be screened for the ability to inhibit IRF-4/AP-1 family member interaction, and a mixture of IRF-4 or an active fragment thereof and AP-1 family member or an active fragment thereof for a period of time and under conditions sufficient for IRF-4/AP-1 family member interaction; and b) determining the extent of IRF-4/AP-1 family member interaction relative to an otherwise identical reaction mixture which does not include said candidate agent, wherein a decrease in the interaction relative to that of the otherwise identical reaction mixture is indicative of said candidate agent having the ability to inhibit IRF-4/AP-1 family member interaction. In one embodiment, the AP-1 family member is BATF. In certain embodiments, the agent is a small molecule. In certain other embodiments, the agent is a peptidomimetic. In a particular embodiment, the IRF-4/AP-1 interaction is detected by electromobility shift assay (EMSA). In certain embodiments, DNA probes may be added to the reaction mixture comprising the agent to be screened, IRF-4 or an active fragment thereof, and AP-1 family member or an active fragment thereof. In one embodiment, the DNA probes comprise IRF and AP-1 motifs with different spacing requirements, e.g., 0 or 4 bp. In certain embodiments, the DNA probes comprising IRF and AP-1 motifs with different spacing requirements are derived from intronic regions within the CTLA-4 and Bcl11b genes. In some embodiments, the IRF site is TTTC. In certain embodiments, the AP-1 site is TGACTCA. In certain other embodiments, the AP-1 site is TGAGTCA. In a particular embodiment, an IRF/AP1 inhibitor may be screened using the electromobility shift assay (EMSA) as described in Example 3 herein. In one embodiment, the present invention provides methods of treating a Th17-mediated disease comprising administering to a subject in need an agent identified by the methods of the invention.

In one aspect, the present invention provides an agent identified by the methods of the invention. In one embodiment, the agent is a small molecule. In another embodiment, the agent is a peptidomimetic.

In another aspect, the present invention provides methods of treating a Th17-mediated disease comprising administering to a subject in need an agent that inhibits the interaction of IRF-4 and an AP-1 family member. In one embodiment, the AP-1 family member is BATF. In some embodiments, the Th17 mediated disease is selected from the group consisting of multiple sclerosis, rheumatoid arthritis, inflammatory bowel disease, and psoriasis. In a specific embodiment, the Th17 mediated disease is multiple sclerosis. In another embodiment, the Th17 mediated disease is rheumatoid arthritis. In yet another embodiment, the Th17 mediated disease is inflammatory bowel disease. In still another embodiment, the Th17 mediated disease is psoriasis. In certain embodiments, the subject is a human patient. In yet another aspect, the invention concerns the use of an agent that inhibits the interaction of IRF-4 and an AP-1 family member, e.g., BATF, in the preparation of a medicament for the treatment of a Th17-mediated disease. In a further aspect the invention concerns an agent that inhibits the interaction of IRF-4 and an AP-1 family member, e.g., BATF, for use in the treatment of a Th17-mediated disease.

In another aspect, the present invention provides methods of inhibiting the recruitment of IRF-4 to an AP-1/IRF composite motif by a DNA-bound BATF/JunB heterodimer.

In one aspect, the present invention provides methods of inhibiting the recruitment of IRF-4 to an AP-1/IRF composite motif by a DNA-bound BATF/c-Jun heterodimer.

In still another aspect, the present invention provides methods of manufacturing an agent capable of inhibiting the IRF4 and AP-1 family member interaction.

In another aspect, the instant invention provides an isolated molecular complex comprising IRF-8 and an AP-1 family member. In some embodiments, the present invention provides an isolated molecular complex consisting of IRF-8 and an AP-1 family member. In certain other embodiments, the present invention provides an isolated molecular complex consisting essentially of IRF-8 and an AP-1 family member. In certain embodiments, the AP-1 family member is BATF. In some embodiments, the isolated molecular complex comprises an additional AP-1 family member. In one embodiment, the additional AP-1 family member is JunB. In other embodiments, the molecular complexes comprises IRF-8 with BATF and JunB.

In certain embodiments, the IRF/AP-1 composite motif comprises an IRF site and an AP-1 site. In one embodiment, the IRF/AP-1 composite motif consists of an IRF site and an AP-1 site. In another embodiment, the IRF/AP-1 composite motif consists essentially of an IRF site and an AP-1 site. In one embodiment, the AP-1/IRF composite motif comprises a sequence provided in FIG. 1d of Example 2A. In further embodiments, the molecular complex of the invention is capable of binding an AP-1/IRF composite motif comprising an IRF site and an AP-1 site. In yet further embodiments, the molecular complex of the invention is capable of binding an AP-1/IRF composite motif consisting of an IRF site and an AP-1 site. In still further embodiments, the molecular complex of the invention is capable of binding an AP-1/IRF composite motif consisting essentially of an IRF site and an AP-1 site. In one embodiment, the AP-1/IRF composite motif comprises a 4-bp space between the IRF site and the AP-1 site. In one embodiment, the AP-1/IRF composite motif comprises the sequence TTTC(N4)TGA(G/C)T(C/A)A. In another embodiment, the AP-1/IRF composite motif comprises a 3-bp space between the IRF site and the AP-1 site. In still another embodiment, the AP-1/IRF composite motif comprises a 2-bp space between the IRF site and the AP-1 site. In a further embodiment, the AP-1/IRF composite motif comprises a 1-bp space between the IRF site and the AP-1 site. In another embodiment, the AP-1/IRF composite motif comprises no space between the IRF site and the AP-1 site. In one embodiment, the AP-1/IRF composite motif comprises the sequence GAAATGA(G/C)T(C/A)A. In a specific embodiment, the molecular complex of the invention is capable of binding an AP-1/IRF composite motif where the IRF site is TTTC and the AP-1 site is TGA(C/G)TCA.

### BRIEF DESCRIPTION OF THE DRAWINGS

FIG. 1 shows ChIPseq analysis of the IRF4 cistrome in CD4+ T-helper cells polarized under Th0 and Th17 conditions that reveals an overrepresentation of AP-1 motifs. Naïve CD4+, CD62L+, CD25− T-cells were cultured under Th0 or Th17 polarizing conditions. (a) Time course analysis of IRF4 expression. IRF4 is induced upon activation of CD4+ T-cells (Th0) and their differentiation into Th17 cells. (b) Genomic distribution of the IRF4 target sites in Th0 and Th17 cells represented as % of total sites. Th0 or Th17 cells (42h) were treated with formaldehyde to cross-link chromatin. Cross-linked and sheared chromatin was immunoprecipitated with aIRF4 antibodies and the eluted DNA was processed for Next-Gen sequencing. 241 IRF4 target sites were identified in Th0 cells vs 2333 target sites in Th17 cells using QuEST. Cis-regulatory Element Annotation System (CEAS) was used to analyze the genomic distribution of the target sites. (c) De Novo motif analysis of IRF4 target sequences in Th0 and Th17 cells. The target sequences associated with the IRF4 peaks (241 for Th0 and 2333 for Th17) were analyzed using MEME to identify overrepresented motifs within +/−100 bp of the peak maxima. The sequence logos depict the AP-1/IRF composite and AP-1 motifs with their respective frequencies that are enriched in the IRF4 target sequences in the Th0 and Th17 cells chromatin.

FIG. 2 shows BATF cistrome in Th17 cells is enriched for composite AP-1-IRF motifs that bind IRF4/BATF/JunB complexes. (a) J558L B cells were subjected to chromatin crosslinking and immunoprecipitation with antibodies directed against IRF4. After deep sequencing and alignment of reads to the mouse genome, QuEST was used to obtain positions of specific enrichment (peaks). Peaks were analyzed with the MEME algorithm. The most prevalent motif is formatted in Logo and the nature of the transcription factor binding site(s) is indicated along with the frequency of its occurrence in the cistrome. (b) Immunoblot analysis of IRF4 expression in activated CD4+ T cells (antiCD3 and antiCD28) cultured under Th17 polarizing conditions. Actin served as a loading control. (c) Th17 cells, differentiated in vitro (42 hours), were subjected to ChIPseq analysis as described in FIG. 3a using antibodies directed against BATF. Highly represented motifs within the BATF cistrome (peaks) are depicted. (d, e) EMSAs using nuclear extracts from Th17 cells and the indicated AICE motif probes as in FIG. 3d. Complexes were supershifted with antibodies indicated above each lane. (f) EMSAs using nuclear extracts from Th17 cells and the indicated wild type or mutant AICE motif probes as in FIG. 1e. Data in panels d-f are representative of at least two independent experiments.

FIG. 3 shows IRF-4 and BATF cistromes in Th17 cells are enriched for composite AP-1-IRF motifs that direct cooperative binding. Th17 cells differentiated in vitro (42 hours) were subjected to chromatin crosslinking and immunoprecipitation with antibodies directed against IRF-4 or BATF. After deep sequencing and alignment of reads to the mouse genome (mm9), QuEST was used to compile the alignments and obtain positions of specific enrichment (peaks). Overrepresented sequence motifs were analyzed using the MEME algorithm. (a) Highly represented motifs within the IRF-4 cistrome (2,333 peaks). The motifs are formatted in Logo and the transcription factor binding site(s) is indicated along with the frequency of occurrence in the cistrome. (b) Union analysis of the IRF-4 and BATF cistromes. Numbers indicate unique or coincident peaks, the latter within 100 bp of each other. (c) Highly represented motifs within the coincident binding peaks of IRF-4 and BATF (1,936 peaks). (d-e) EMSAs using nuclear extracts from 293T cells transiently transfected with plasmids expressing IRF-4, BATF or JunB. Protein composition of each binding reaction is indicated below the lane. Complexes were supershifted with antibodies indicated above each lane (d). The AP-1 (red nucleotides) and IRF sites (green nucleotides) are indicated within each sequence. The sequences were derived from IRF-4 and BATF co-targeted regions within the CTLA-4 or Bcl11b loci. (e) EMSAs utilizing wild type or mutant AICE motifs. CTLA-4 or Bcl11b DNA probes containing base substitution mutations in either the IRF site (IRF mut), the AP-1 site (AP-1 mut) or both (IRF/AP-1 mut) (Table 2) were used in indicated binding reactions. Red and green arrows mark the positions of the BATF/JunB/DNA and IRF-4/BATF/JunB/DNA complexes respectively. Data are representative of at least two independent experiments.

FIG. 4 shows IRF4 and BATF cooperatively bind to composite IRF/AP-1 motifs. (a) DNA sequences of probes used for gel electromobility shift assays. These sequences were derived from genes co-targeted by IRF-4 and BATF and harbor differing spacing of the IRF and AP-1 motifs. (b-e) Electromobility shift assays using nuclear extracts isolated from either 293T cells transfected with His-tagged IRF4 or Flag-tagged BATF expressing plasmids (b,c) or in-vitro differentiated Th17 cells (42 hours) (d,e). Positions of various protein/DNA complexes are indicated. Nuclear extracts were incubated with either the wild type Bcl11b or CTLA-4 probes or their indicated mutant counterparts in which either the IRF or AP-1 site or both were altered.

FIG. 5 shows assembly of IRF-4/BATF/JunB complexes on presumptive regulatory sequences in key Th17 genes. (a) ChIPseq tracks for IRF-4 (green) or BATF (red) at the Il17a, Il21, Il23r and Il12rb1 loci. The tag enrichment (y-axis) is displayed as a histogram using the UCSC genome browser display. Scale bars (kb) are indicated. Coincident peaks containing AICE motifs are highlighted (boxed regions). (b) ChIP analysis with IRF-4, BATF and JunB antibodies in Th17 cells. The indicated sequences span IRF-4/BATF coincident binding peaks displayed in the boxed regions in panel a. The negative control sequence was from an intron in the Rorc locus that is not targeted by IRF-4 or BATF. The CTLA-4 co-bound region served as a positive control. (c) EMSAs using nuclear extracts from Th17 cells and the indicated DNA probes and antibodies (see FIG. 3d, e for labels). Data are representative of at least two independent experiments.

FIG. 6 shows assembly of IRF4/BATF/JunB complexes on presumptive regulatory sequences in key Th17 genes. EMSAs using the indicated probes (see FIG. 2c) and nuclear extracts from 293T cells transfected with plasmids encoding the indicated proteins (a, b, d and e) or from in vitro differentiated Th17 cells (42 hours) (c). Complexes were supershifted with the indicated antibodies (a, c, d). The green arrow indicates position of the predominant AP1DNA complexes containing IRF4, whereas the red arrow indicates AP1 complexes lacking IRF4. For panels b and e, indicated probes contained mutations in either the IRF site (IRF mut), the AP1site (AP1 mut) or both (IRF/AP1 mut). Sequences of various DNA probes are detailed in Table 2. Data are representative of at least two independent experiments.

FIG. 7 shows IRF4 regulated genes that are co-bound by IRF4/BATF complexes. (a) Microarray analysis of Irf4^{+/-} or Irf4^{-/-}CD4⁺ T cells differentiated under Th17 conditions for 42 hours. Hierarchical clustering was performed on genes that were differentially expressed by at least 3 fold using the Partek genomics suite. Select biologically relevant genes are indicated on the left. (b) RTPCR analysis of key genes in Irf4^{+/-} or Irf4^{-/-}CD4⁺ T cells differentiated as above. (c) Examples of IRF4 regulated and IRF4/BATF cobound genes that contain AICE motifs (d) ChIP analysis of BATF binding to indicated AICE motif sequences in Irf4^{+/-} or Irf4^{-/-}CD4⁺ T cells differentiated under Th17 conditions for 42 hours.

FIG. 8 shows network of IRF-4 regulated genes in Th17 cells that are co-targeted by BATF. (a) Network analysis of genes positively regulated by IRF-4 and co-targeted by IRF-4 and BATF. Genes that were significantly positively regulated by IRF-4 (<−2-Fold change, p<0.05) and co-targeted by IRF-4/BATF (coincident peaks within 50 kb of TSS or nearest gene) were subject to pathway analysis using IPA (Ingenuity® Systems). Of 154 such genes, 65 interconnected genes (by either positive or negative gene-regulation or protein-protein interactions) are shown and depicted in blue. The network of genes is in turn highly connected with key Th17 transcription factors, Rorγt, AHR and STAT3. Connections between these TFs and the genes in the network are shown in orange. (b) Biologically relevant genes from the network analysis in (a) are grouped into three modules—T-cell activation, Th17 and TGF-β signaling. Positive regulatory connections (->) and protein-protein interactions (--) are indicated. (c) Activated CD4 T cells (anti-CD3 and anti-CD28) cultured under non-polarizing conditions (Th0) were transduced with IRF-4-IRES-GFP and/or BATF-IRES-hCD4 expressing retroviruses. Four days after infection cells were stimulated with PMA and Ionomycin for 4 h and analyzed for intracellular CTLA-4 protein by flow cytometry. (d) Mean fluorescence intensities of CTLA-4 expression corresponding to the three experimental conditions after normalization to their corresponding controls. (e) Activated CD4+ T cells cultured under polarizing conditions (Th17) were transduced with retroviruses described in panel c. Four days after infection, cells were stimulated with PMA and Ionomycin for 4 h and then analyzed for IL-17A protein by flow cytometry. (f) Percentage of IL-17A expressing cells under each condition after normalization to their corresponding controls. Data in panels c and e are from three independent experiments (average and s.d.). p-values were calculated with one-way ANOVA: * represents a p-value of ≦0.05).

FIG. 9 shows specificity of AP-1/IRF complexes that cooperatively assemble on AICE motifs. (a-d) EMSAs using the Bcl11b probe and 293T nuclear extracts as a source of indicated proteins (a) c-Jun, BATF, IRF-4, (b) FosL2, JunB, IRF-4, (c) JunB, BATF, IRF-8 or (d) JunB, BATF, IRF-3 (e) EMSA using an ISRE containing probe and IRF-3 or IRF-4. Complexes were supershifted with indicated antibodies. Red and green arrows mark the ternary and quaternary protein-DNA complexes, respectively as in FIG. 1d. Data are representative of at least two independent experiments. (f-h) Th0 cells and Th2 cells differentiated in vitro (42 hours) and LPS-activated dendritic cells (6 hours) were subjected to chromatin crosslinking and immunoprecipitation with antibodies directed against IRF-4. Immunoprecipitated chromatin was deep sequenced and analyzed as in FIG. 3a. Highly represented motifs within the IRF-4 cistrome in Th0 (263 peaks), in Th2 (797 peaks) and in dendritic cells (10364 peaks) are displayed.

FIG. 10 shows specificity of AP1/IRF complexes that cooperatively assemble on AICE motifs. EMSAs using the indicated probes and 293T nuclear extracts as a source of indicated proteins: (a) c-Jun, BATF, IRF4, (b) FosL2, JunB, IRF4, (c) JunB, BATF, IRF8, (d, e) JunB, BATF and the DBD (aa 2139) of IRF4. (f, g) Molecular modeling using PYMOL based on the crystal structure of the IRF4 DBD/PU.1DNA complex and the crystal structure of the IRF3 DBD/ATF2/c-Jun-DNA complex (residues 336-396 of ATF2 and 253-314 of c-Jun), applying it to the homologous residues in BATF (amino-acids: 2090) and JunB (as: 267328) on two types of AICE motifs (Bcl11b, 0 bp spacing and CTLA4, 4 bp spacing). See Materials and Methods for additional details.

FIG. 11 shows IFR4 and BATF cooperatively induce the expression of the ICOS and CTLA-4 genes. (a,b) Th0 cells were transduced with IRF4 and/or BATF retroviral vectors and analyzed by FACS for either cell surface expression of ICOS (two days after activation) or intracellular CTLA-4 levels after re-stimulation with PMA and Ionomycin (four days after activation). (a) A representative experiment is shown. (b) MFI (mean fluorescence intensity) of ICOS or CTLA4 expression promoted by ectopic expression of IRF4 and/or BATF, based on three independent experiments.

### DETAILED DESCRIPTION OF THE INVENTION

### I. Definitions

As used herein, the term “IRF-4” (also referred to interchangeably as pip, MUM1, LSIRF, NFEM5, and ICSAT) is intended to refer to an Interferon Regulatory Factor (IRF) family member. The term also encompasses naturally occurring variants of IRF-4, e.g., splice variants or allelic variants. The nucleic acid sequence of an exemplary murine IRF-4 is shown in SEQ ID NO: 164. The amino acid sequence of an exemplary murine IRF-4 is shown in SEQ ID NO:165. The amino acid sequence of an exemplary human IRF-4 is shown below:

MNLEGGGRGGEFGMSAVSCGNGKLRQWLIDQIDSGKYPGLVWENEEKSI
FRIPWKHAGKQDYNREEDAALFKAWALFKGKFREGIDKPDPPTWKTRLR
CALNKSNDFEELVERSQLDISDPYKVYRIVPEGAKKGAKQLTLEDPQMS
MSHPYTMTTPYPSLPAQQVHNYMMPPLDRSWRDYVPDQPHPEIPYQCPM
TFGPRGHHWQGPACENGCQVTGTFYACAPPESQAPGVPTEPSIRSAEAL
AFSDCRLHICLYYREILVKELTTSSPEGCRISHGHTYDASNLDQVLFPY
PEDNGQRKNIEKLLSHLERGVVLWMAPDGLYAKRLCQSRIYWDGPLALC
NDRPNKLERDQTCKLFDTQQFLSELQAFAHHGRSLPRFQVTLCFGEEFP
DPQRQRKLITAHVEPLLARQLYYFAQQNSGHFLRGYDLPEHISNPEDYH
RSIRHSSIQE (Genbank Accession No. NP_002451.2)

As used herein, the term “IRF-8” (also referred to interchangeably as Interferon regulatory factor 8, IRF-8, IRF8, H—ICSBP, ICSBP, 1CSBP I, Interferon consensus sequence-binding protein) is intended to refer to an Interferon Regulatory Factor (IRF) family member. The term also encompasses naturally occurring variants of IRF-8, e.g., splice variants or allelic variants. The nucleic acid sequence of an exemplary murine IRF-8 is shown in SEQ ID NO: 166. The amino acid sequence of an exemplary murine IRF-8 is shown in SEQ ID NO:167. The amino acid sequence of an exemplary human IRF-8 is shown below:

MCDRNGGRRLRQWLIEQIDSSMYPGLIWENEEKSMFRIPWKHAGKQDYNQ
EVDASIFKAWAVFKGKFKEGDKAEPATWKTRLRCALNKSPDFEEVTDRSQ
LDISEPYKVYRIVPEEEQKCKLGVATAGCVNEVTEMECGRSEIDELIKEP
SVDDYMGMIKRSPSPPEACRSQLLPDWWAQQPSTGVPLVTGYTTYDAHHS
AFSQMVISFYYGGKLVGQATTTCPEGCRLSLSQPGLPGTKLYGPEGLELV
REPPADAIPSERQRQVTRKLFGHLERGVLLHSSRQGVFVKRLCQGRVECS
GNAVVCKGRPNKLERDEVVQVFDTSQFFRELQQFYNSQGRLPDGRVVLCF
GEEFPDMAPLRSKLILVQIEQLYVRQLAEEAGKSCGAGSVMQAPEEPPPD
QVERMFPDICASHQRSFFRENQQITV
(Genbank Accession No. NP_002154.1)

As used herein, the term “AP-1 family member” (also referred to interchangeably as simple “AP-1”) is intended to refer to a protein that is a member of the AP-1 family of transcription factors, examples of which include, but are not limited to, BATF, c-Jun, c-Fos, Fra-1, Fra-2, Jun B and Jun D. The nucleotide and predicted amino acid sequences of AP-1 family proteins are known in the art. For example, the nucleotide and predicted amino acid sequences of human c-fos are disclosed in van Straaten, F. et al. (1983) Proc. Natl. Acad. Sci. USA 80:3183-3187. The nucleotide and predicted amino acid sequences of human c-jun are disclosed in Bohmann, D. et al. (1987) Science 238:1386-1392. The nucleotide and predicted amino acid sequences of human jun-B and jun-D are disclosed in Nomura, N. et al. (1990) Nucl. Acids Res. 18:3047-3048. The nucleotide and predicted amino acid sequences of human fra-1 and fra-2 are disclosed in Matsui, M. et al. (1990) Oncogene 5:249-255. For example, the amino acid sequence of an exemplary BATF is shown below:

MPHSSDSSDSSFSRSPPPGKQDSSDDVRRVQRREKNRIAAQKSRQRQTQK
ADTLHLESEDLEKQNAALRKEIKQLTEELKYFTSVLNSHEPLCSVLAAST
PSPPEVVYSAHAFHQPMVSSPRFQP
(Genbank Accession No. NP_006390)

For example, the amino acid sequence of an exemplary c-Jun is shown below:

MTAKMETTFYDDALNASFLPSESGPYGYSNPKILKQSMTLNLADPVGSLK
PHLRAKNSDLLTSPDVGLLKLASPELERLIIQSSNGHITTTPTPTQFLCP
KNVTDEQEGFAEGFVRALAELHSQNTLPSVTSAAQPVNGAGMVAPAVASV
AGGSGSGGFSASLHSEPPVYANLSNFNPGALSSGGGAIPSYGAAGLAFPA
QPQQQQQPPHHLPQQMPVQHPRLQALKEEPQTVPEMPGETPPLSPIDMES
QERIKAERKRMRNRIAASKCRKRKLERIARLEEKVKTLKAQNSELASTAN
MLREQVAQLKQKVMNHVNSGCQLMLTQQLQTF
(Genbank Accession No. AAH68522.1)

For example, the amino acid sequence of an exemplary jun-B is shown below:

MCTKMEQPFYHDDSYTATGYGRAPGGLSLHDYKLLKPSLAVNLADPYRSL
KAPGARGPGPEGGGGGSYFSGQGSDTGASLKLASSELERLIVPNSNGVIT
TTPTPPGQYFYPRGGGSGGGAGGAGGGVTEEQEGFADGFVKALDDLHKMN
HVTPPNVSLGATGGPPAGPGGVYAGPEPPPVYTNLSSYSPASASSGGAGA
AVGTGSSYPTTTISYLPHAPPFAGGHPAQLGLGRGASTFKEEPQTVPEAR
SRDATPPVSPINMEDQERIKVERKRLRNRLAATKCRKRKLERIARLEDKV
KTLKAENAGLSSTAGLLREQVAQLKQKVMTHVSNGCQLLLGVKGHAF
(Genbank Accession No. NP_002220.1)

As used herein, the term “AP-1/IRF composite motif” or “RICE” is intended to refer to a heterogeneous DNA sequence which allows co-binding of AP-1 and IRF.

As used herein, the term “transcription factor” is intended to refer to a factor (e.g., a protein) that acts in the nucleus to regulate the transcription of a gene. The term “transcription factor” is intended to include factors that directly regulate transcription (e.g., have intrinsic transcriptional activation or inhibitory activity) and factors that indirectly regulate transcription (e.g., through interaction with other factors that have intrinsic transcriptional activation or inhibitory activity).

The term “agent” as used herein means any compound or substance such as, but not limited to, a small molecule, nucleic acid, polypeptide, peptide, drug, ion, etc. An “agent” can be any chemical, entity, or moiety, including without limitation synthetic and naturally-occurring proteinaceous and non-proteinaceous entities. In some embodiments, an agent is a nucleic acid, a nucleic acid analogue, protein, antibody, peptide, aptame, oligomer of nucleic acids, amino acid, or carbohydrate, and includes, without limitation, proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties include unsubstituted or substituted alkyl, aromatic, or heterocyclyl moieties. Compounds can be known to have a desired activity and/or property, or can be selected from a library of diverse compounds.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “screening” as used herein refers to the use of cells, tissues, or derivatives of them in the laboratory to identify agents with a specific function, e.g., a modulating activity. In some embodiments, described herein are screening methods to identify agents (e.g., compounds or drugs) that inhibit or otherwise modulate IRF-4/AP-1 interaction.

The term “library,” as used herein, refers to a mixture of heterogeneous agents, such as, for example, small molecules, polypeptides or nucleic acids. The library may be composed of members, each of which have a single small molecule, polypeptide or nucleic acid sequence. Structural and/or sequence differences between library members are responsible for the diversity present in the library. The library can take the form of a simple mixture of small molecules, polypeptides or nucleic acids, or can be in the form of organisms or cells, for example bacteria, viruses, animal or plant cells and the like, transformed with a library of e.g., nucleic acids. Preferably, each individual organism or cell contains only one or a limited number of library members. Advantageously, the nucleic acids are incorporated into expression vectors, in order to allow expression of the polypeptides encoded by the nucleic acids. Therefore, in some embodiments, a library can take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

A “marker” as used herein is used to describe the characteristics and/or phenotype of a cell, e.g., a Th17 cell marker. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in or on the surface of a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to express or produce one or more specific cytokines or chemokines, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics. When a marker is a protein receptor or other such molecule expressed on the surface of a cell, it is termed herein as a “cell-surface marker.”

The term “modulate” is used consistently with its use in the art, e.g., meaning to cause or facilitate a qualitative or quantitative change, alteration, or modification in one or more biological processes, mechanisms, effects, responses, functions, activities, pathways, or other phenomena of interest. Without limitation, such change may be an increase, decrease, or change in relative strength or activity of different components or branches of the process, mechanism, effect, response, function, activity, pathway, or phenomenon. Accordingly, as used herein “modulating” refers to a change of at least 10%, at least about 20%, at least about 30%, at least about 40%, at least about 50%, at least about 60%, at least about 70%, at least about 80%, at least about 90%, up to and including a 100% change, or any change of at least about 2-fold, at least about 3-fold, at least about 4-fold, at least about 5-fold, at least about 10-fold, at least about 100-fold, at least about 1000-fold, or any modulation between 2-fold and 1000-fold, or greater, as compared to a reference level. A “modulator” is an agent, such as a small molecule or other agents described herein, that causes or facilitates a qualitative or quantitative change, alteration, or modification in a process, mechanism, effect, response, function, activity, pathway, or phenomenon of interest.

The term “Th17-mediated disease” is used herein in the broadest sense and includes all diseases and pathological conditions the pathogenesis of which involves abnormalities of Th17 cells. Non-limiting examples of Th17-mediated diseases include multiple sclerosis, rheumatoid arthritis, inflammatory bowel disease, and psoriasis.

The term “inflammatory disease” and “inflammatory disorder” are used interchangeably and mean a disease or disorder in which a component of the immune system of a mammal causes, mediates or otherwise contributes to an inflammatory response contributing to morbidity in the mammal. Also included are diseases in which reduction of the inflammatory response has an ameliorative effect on progression of the disease. Included within this term are immune-mediated inflammatory diseases, including autoimmune diseases.

An “autoimmune disorder” or an “autoimmune disease” as the terms are used herein refer to those disorders or diseases that are the result of inappropriate activation of immune cells that are reactive against self tissue, and which are characterized by the production of cytokines, such as IL-17, and autoantibodies involved in the pathology of the diseases. Preventing the activation or effector function, such as IL-17 production, of autoreactive immune cells can reduce or eliminate disease symptoms. Accordingly, in some embodiments, an autoreactive immune cell is a an autoreactive Th17 cell. Non-limiting examples of autoimmune diseases include multiple sclerosis, rheumatoid arthritis, Crohn's disease, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma (e.g., with anti-collagen antibodies), mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Most autoimmune diseases are also encompassed within the term “chronic inflammatory diseases.” Such diseases or disorders are processes associated with long-term (>6 months) activation of inflammatory immune cells, such as Th17 cells. The chronic inflammation leads to damage of patient organs or tissues. In addition to autoimmune disorders, many diseases are considered to be chronic inflammatory disorders, but are not currently known to have an autoimmune basis. Examples include atherosclerosis, congestive heart failure, polyarteritis nodosa, Whipple's Disease, and primary sclerosing cholangitis.

The term “mammal” for the purposes of treatment refers to any animal classified as a mammal, including but not limited to, humans, rodents, sport, zoo, pet and domestic or farm animals such as dogs, cats, cattle, sheep, pigs, horses, and non-human primates, such as monkeys. Preferably the rodents are mice or rats. Preferably, the mammal is a human, also called herein a patient.

As used herein, “treating” describes the management and care of a mammal for the purpose of combating any of the diseases or conditions targeted in accordance with the present invention, including, without limitation, inflammatory bowel disease or a related condition, and includes administration to prevent the onset of the symptoms or complications, alleviate the symptoms or complications of, or eliminate the targeted diseases or conditions.

The term “antibody” is used in the broadest sense and specifically covers monoclonal antibodies (including antagonist, e.g. neutralizing antibodies and agonist antibodies), polyclonal antibodies, multi-specific antibodies (e.g., bispecific antibodies), as well as antibody fragments. The monoclonal antibodies specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 [1984]). The monoclonal antibodies further include “humanized” antibodies or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat or rabbit having the desired specificity, affinity, and capacity. In some instances, Fv FR residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); and Reichmann et al., Nature, 332:323-329 (1988). The humanized antibody includes a PRIMATIZED® antibody wherein the antigen-binding region of the antibody is derived from an antibody produced by immunizing macaque monkeys with the antigen of interest.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10):1057-1062 (1995)); single-chain antibody molecules; and multispecific antibodies formed from antibody fragments.

An “isolated” polypeptide or “isolated molecular complex” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the polypeptide, including antibodies, will be purified (1) to greater than 95% by weight of the antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated compound, e.g. antibody or other polypeptide, includes the compound in situ within recombinant cells since at least one component of the compound's natural environment will not be present. Ordinarily, however, isolated compound will be prepared by at least one purification step.

“Chronic” administration refers to administration of the agent(s) in a continuous mode as opposed to an acute mode, so as to maintain the desired effect for an extended period of time.

“Intermittent” administration is treatment that is not consecutively done without interruption, but rather is cyclic in nature.

Administration “in combination with” one or more further therapeutic agents includes simultaneous (concurrent) and consecutive administration in any order.

### II. Detailed Description

IRF-4 and IRF-8 are evolutionarily diverged members of the IRF family of transcription factors (T. Tamura, et al. Annual review of Immunology 26, 535 (2008).). Unlike other members, which are ubiquitously expressed, IRF-4 and -8 are restricted to the immune system and play key roles in the differentiation and functioning of innate and adaptive immune cells (M. Lohoff, T. W. Mak. Nature Reviews. Immunology 5, 125 (2005); N. S. De Silva, et al. Immunological Reviews 247, 73 (2012). IRF-4 is required for B cells to undergo class switch recombination and plasma cell differentiation (U. Klein et al. Nature Immunology 7, 773 (2006); R. Sciammas et al. Immunity 25, 225 (2006), whereas IRF-8 is needed for macrophage development (T. Tamura, et al. Immunity 13, 155 (2000). Both transcription factors regulate dendritic cell differentiation (T. Tamura et al. J Immunol 174, 2573 (2005). IRF-4 additionally regulates the generation and/or functioning of various types of helper T cells including Th17 (A. Brustle et al. Nature Immunology 8, 958 (2007)), Th2 (M. Lohoff et al., PNAS 99, 11808 (2002); J. Rengarajan et al., The Journal of Experimental Medicine 195, 1003 (2002)), Tfh (N. Bollig et al., PNAS, Epub ahead of print (2012)) and iTregs (Y. Zheng et al., Nature 458, 351 (2009)). The molecular mechanisms by which IRF-4 and -8 function in programming diverse patterns of gene expression in the fore-mentioned immune cells, particularly those of the T-lineage are poorly understood.

In distinction with other members of the IRF family, IRF-4 and -8 bind with low affinity to interferon-stimulated response elements (ISREs) (C. Bovolenta et al., PNAS 91, 5046 (1994); T. Matsuyama et al., Nucleic Acids Research 23, 2127 (1995).) These sequences (GAAANNGAAA) (SEQ ID NO: 1) contain a dimeric GAAA core motif that is specifically contacted by the IRF DNA binding domain (C. R. Escalante et al., Molecular Cell 10, 1097 (2002).) IRF-4 and -8 appear to have evolved to interact with other transcription factors so as to facilitate their recruitment to alternate genomic regulatory elements. The Ets family transcription factors, PU.1 and Spi-B, represent interaction partners that are best characterized (A. L. Brass, et al., Genes & Development 10, 2335 (1996); A. L. Brass, et al., The EMBO Journal 18, 977 (1999); C. F. Eisenbeis, et al., Genes & Development 9, 1377 (1995)). They are able to recruit IRF-4 or -8 to composite Ets-IRF motifs (EICE) that are comprised by the canonical sequence GGAANNGAAA (SEQ ID NO: 2) (A. L. Brass, et al., Genes & Development 10, 2335 (1996); C. F. Eisenbeis, et al., Genes & Development 9, 1377 (1995)). IRF-4 has also been shown to interact with NFATc and this complex is implicated in the regulation of the IL-4 gene in Th2 cells (J. Rengarajan et al., The Journal of Experimental Medicine 195, 1003 (2002).) However, the generality of this mode of DNA recruitment and the biochemical mechanism underlying assembly on DNA remains to be elucidated. ChIPseq analysis of IRF-4 in IL-21 stimulated T cells has revealed co-binding with STAT3 (H. Kwon et al., Immunity 31, 941 (2009).) The mechanism underlying co-targeting of IRF-4 and STAT3 to genomic regions remains to be defined but does not appear to involve DNA dependent cooperative binding. Thus in T helper cells, such as Th17 cells, that do not express PU.1 or Spi-B, it remains to be determined how IRF-4 and -8 are recruited to their various genomic targets.

IL-17 producing T-helper (Th17) cells are a subset of T helper cells that function in host defense by producing the pro-inflammatory cytokines IL-17 and TNFα. (reviewed in Zhou and Littman, Hirahara et al.,). They function in clearance of extracellular pathogens and also manifest a major pathologic role in a variety of experimentally induced autoimmune diseases, such as colitis, encephalomyelitis and psoriasis (G. J. Martinez, et al., Annals of the New York Academy of Sciences 1143, 188 (2008); D. R. Littman, A. Y. Rudensky, Cell 140, 845 (2010); A. Peters, Y. Lee, V. K. Kuchroo, Current Opinion in Immunology 23, 702 (2011).) Genome-wide analysis reveals that the gene expression pattern of differentiating Th17 cells diverges significantly from that of Th0 and Th2 cells. Irf4−/− T cells are defective in undergoing Th17 differentiation and fail to express IL-17, IL-22, IL-21 and IL-23R (A. Brustle et al., Nature Immunology 8, 958 (2007); N. Bollig et al., Transcription factor IRF4 determines germinal center formation through follicular T-helper cell differentiation. PNAS, Epub ahead of print (2012); M. Huber et al., PNAS 105, 20846 (2008).) Consequently, Irf4−/− mice are protected against experimental autoimmune encephalomyelitis (EAE) and colitis (A. Brustle et al., Nature Immunology 8, 958 (2007); J. Mudter et al., Inflammatory Bowel Diseases 17, 1343 (2011).) Intriguingly, IRF-8 antagonizes Th17 cell differentiation and its loss results in more severe colitis (W. Ouyang, et al., Immunity 28, 454 (2008).) Th17 differentiation depends on a combinatorial set of transcription factors that additionally includes RORγt (Ivanov, II et al., Cell 126, 1121 (2006); X. O. Yang et al., Immunity 28, 29 (2008), STAT3 (A. N. Mathur et al., J Immunol 178, 4901 (2007); X. O. Yang et al., The Journal of Biological Chemistry 282, 9358 (2007)) and BATF (B. U. Schraml et al., Nature 460, 405 (2009).) RORγt is a lineage-specific regulator whose forced expression in activated T cells is sufficient to induce the expression of IL-17A, IL-17F and IL23R (Ivanov, II et al., Cell 126, 1121 (2006); X. O. Yang et al., Immunity 28, 29 (2008).) However, restoring expression of RORγt in Irf-4−/− or Batf−/− T cells results in a partial rescue of the Th17 differentiation program (A. Brustle et al., Nature Immunology 8, 958 (2007); B. U. Schraml et al., Nature 460, 405 (2009).) Therefore, IRF-4 and BATF are likely required for the direct activation of Th17 genes independently of or in concert with RORγt. Consistent with this proposition a BATF/JunB heterodimer has been shown to bind to the IL-17, IL 21 and IL-22 promoters (B. U. Schraml et al., Nature 460, 405 (2009).) The extent of IRF-4 genomic targets in Th17 cells and its mode of recruitment are not delineated.

By using ChIPseq to analyze the mode of genomic targeting of IRF-4, in Th17 cells, we have uncovered a new set of its molecular partners, select members of the AP-1 family, that enable recruitment to previously unrecognized composite AP-1-IRF motifs. Intriguingly, in Th17 cells, IRF4 binds to a large set of target sequences that contain neighboring AP-1 but not Ets motifs, the latter is the case in B cells. Importantly, the AP-1 family member, BATF, that is required for Th17 cell differentiation (Scharml et al., Betz et al.,) is shown to co-target ˜83% of the sequences that IRF-4 occupies in Th17 cells. In vitro analyses reveal that IRF4 and BATF cooperatively bind to two types of novel composite IRF-AP-1 motifs, found in Th17 genes that are co-targeted by these transcription factors, with different spacing requirements. IRF-4 and a BATF/JunB heterodimer assembled cooperatively on structurally distinct AP-1-IRF composite elements (AICE). Analysis of IRF-4/BATF co-bound and IRF-4-regulated genes revealed transcriptional sub-networks underlying Th17 differentiation. The AICE motif greatly expands the molecular activities of IRF-4 in innate and adaptive cells of the immune system. Furthermore, IRF4 and BATF function coordinately to induce expression of several functionally important T helper cell genes and the differentiation of Th17 cells. Thus IRF-4 has evolved to molecularly interact with both Ets and AP-1 family members in regulating immune cell development and function.

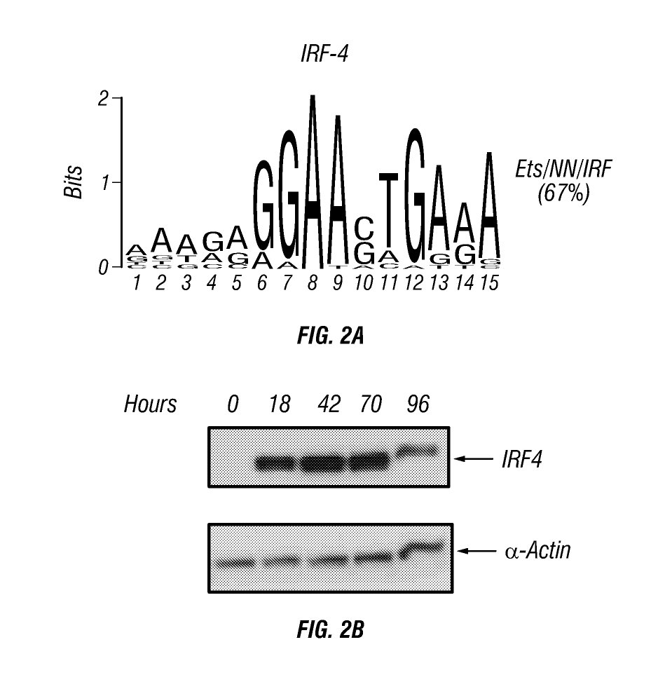

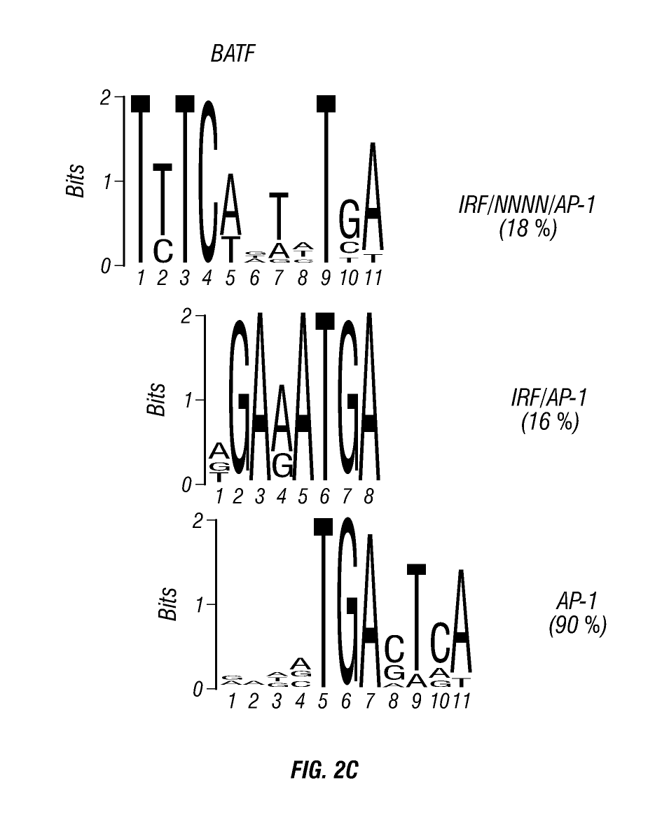

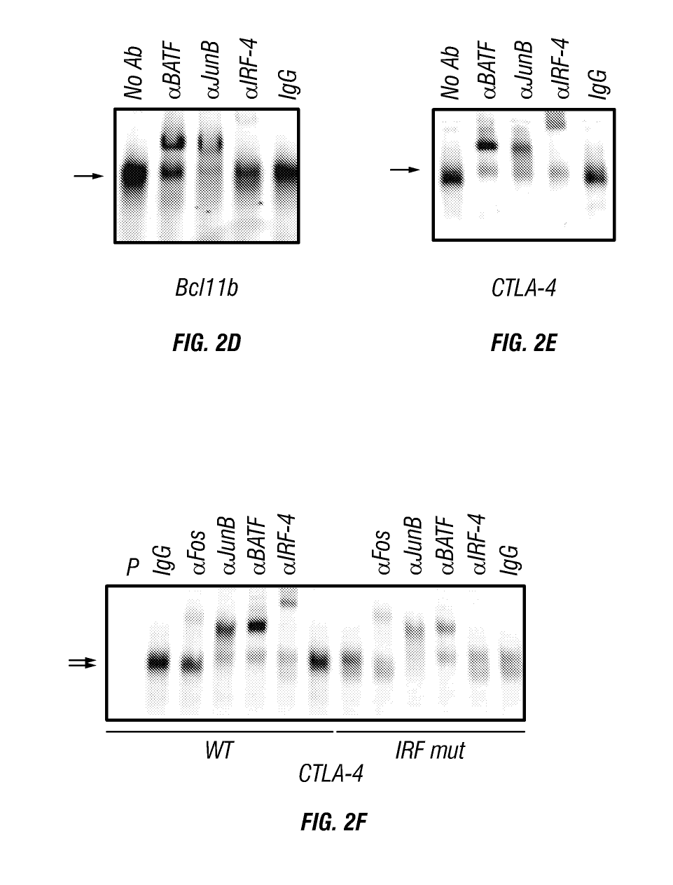

The analysis herein highlights the utility of ChIPseq in revealing new types of composite regulatory DNA elements in the genome and the transcription factors that enable their functioning via cooperative binding. In Th17 cells, AP-1 heterodimers comprising of BATF/JunB or BATF/cJun are likely to represent biologically relevant partners for IRF-4. However they may have differing consequences on gene activity given that cJun appears to be the more potent transactivator (J. Hess, P. Angel, M. Schorpp-Kistner, Journal of Cell Science 117, 5965 (2004)). Finally another AP-1 family member, FosL2 is expressed in an inducible manner in Th17 cells, however a FosL2/JunB heterodimer is unable to recruit IRF-4 to AICE motifs and appears to compete with IRF-4/BATF/JunB for binding to AICE motifs in Th17 nuclear extracts (FIG. 2f). Intriguingly, knockdown of FosL2 in differentiating Th17 cells results in the increased expression of IL-17 (data not shown). Although IRF-4 or IRF-8 can be recruited to AICE motifs by BATF/JunB complexes in vitro, they have opposing functions in Th17 differentiation. This may be due to their ability to recruit distinct transcriptional co-regulators or chromatin modifying complexes. Thus the AICE motif is likely to be a functionally versatile genomic regulatory element whose activity will be dependent on the AP-1 components and the relative levels of IRF-4 and -8 that are expressed in a given cellular context or state.

Based on the results herein, and without being bound by theory, it is proposed that the AICE motif is an immune-specific genomic regulatory element that is widely utilized in both innate and adaptive immune cells. Its functioning is predicted to be confined to cells of the immune system by virtue of the restricted expression of IRF-4 and -8. The involvement of the AICE motif in distinct innate or adaptive immune cell specific programs of gene expression, as exemplified in Th17 cells, is likely to reflect the combinatorial functioning of AP-1/IRF-4, 8 complexes with differentiation state specific regulators such as Rorγt in Th17 cells or Gata-3 in Th2 cells. Given that AP-1 family members and IRF-4 and -8 are signaling induced transcription factors (T. Tamura, H. Yanai, D. Savitsky, T. Taniguchi, Annual review of Immunology 26, 535 (2008); J. Hess, P. Angel, M. Schorpp-Kistner, Journal of Cell Science 117, 5965 (2004)), the AICE motif appears to have evolved to sense and integrate diverse signaling inputs in immune cells. It is capable of integrating such inputs from antigen receptors and their co-receptors as well as cytokine receptors and TLRs. Since AP-1 family members can cooperatively assemble on composite elements with NFAT family proteins (F. Macian, C. Garcia-Rodriguez, A. Rao., The EMBO Journal 19, 4783 (2000)) and juxtaposition of NFAT and IRF sites have been reported on the IL-4 and IL-10 genes (J. Rengarajan et al., The Journal of Experimental Medicine 195, 1003 (2002); C. G. Lee et al., Molecular Immunology 46, 613 (2009)), it will be important to determine if the AP-1/IRF-4, 8 complexes described herein can cooperatively assemble with NFAT proteins thereby enabling the integration of Ca signaling in the regulatory module. It is proposed that the acquisition of the AICE motif by simple variation of AP-1 sites in biologically important immune response genes may have provided a selective force for the evolution of the structurally divergent and specialized members of the IRF family, namely IRF-4 and -8.

The invention is based, at least in part, on experimental findings demonstrating that 1) IRF4 binds to a large set of target sequences with neighboring AP-1 sites; 2) the AP-1 family member BATF binds to many of the same genomic regions as IRF4; 3) IRF4 and BATF cooperatively bind to a representative set of target sequences containing novel IRF/AP-1 composite motifs; 4) IRF4 and BATF cooperatively induce expression of the linked CTLA-4 and ICOS genes that contain a composite IRF/AP-1 motif; and 5) ectopic expression of IRF-4 and BATF in activated T cells under Th17 cell polarizing conditions increases the frequency of IL17A-producing cells. The present invention is also based, at least in part, on experimental data demonstrating that 1) IRF-4 and a BATF/JunB heterodimer assemble cooperatively on structurally distinct AP-1-IRF composite elements (AICE), 2) AICE motifs are associated with genes that comprise transcriptional sub-networks underlying Th17 differentiation, and 3) the AICE motif greatly expands the molecular activities of IRF-4 in innate and adaptive cells of the immune system. Accordingly, described herein are novel compositions and methods for modulating IRF4, AP-1/BATF and Th17 activity and IRF4-, AP-1/BATF- and Th17-mediated inflammatory diseases.

### MODES OF CARRYING OUT THE INVENTION

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

Crystal Structure of IRF-4/AP-1 Family Complexes

Polypeptides, including the IRF-4 and AP-1 family members, have a three-dimensional structure determined by the primary amino acid sequence and the environment surrounding the polypeptide. This three-dimensional structure establishes the activity, stability, binding affinity, binding specificity, and other biochemical attributes of the polypeptide. Thus, knowledge of the three-dimensional structure of a protein provides much guidance in designing agents that mimic, inhibit, or improve its biological activity.

Three-dimensional structures of peptide compounds can be determined in a number of ways, e.g., using nuclear magnetic resonance spectroscopy (NMR) or X-ray crystallography.

Structural information derived from an NMR or peptide crystal structure can be used for the identification of small organic and bioorganic molecules such as peptidomimetics and synthetic organic molecules. An exemplary approach to such a structure based compound design is described in (“Structure Based Drug Design” Pandi Veerapandian, ed. Marcell Dekker, New York 1997).

Many of the most precise methods employ x-ray crystallography (Van Holde, Physical Biochemistry (Prentice Hall: N.J., 1971), pp. 221-239). This technique relies on the ability of crystalline lattices to diffract x-ray or other forms of radiation. Diffraction experiments suitable for determining the three-dimensional structure of macromolecules typically require high-quality crystals. Unfortunately, such crystals have been unavailable for many proteins and protein complexes of interest. Crystals have been described for M-CSF (EP 668,914B1), CD40 ligand (WO 97/00895), and a BC2 Fab fragment (WO 99/01476), for example.

Various methods for preparing crystalline proteins and polypeptides are known in the art (McPherson et al., “Preparation and Analysis of Protein Crystals,” McPherson (Robert E. Krieger Publishing Company, Malabar, Fla., 1989); Weber, Advances in Protein Chemistry, 41: 1-36 (1991); U.S. Pat. Nos. 4,672,108 and 4,833,233). Although there are multiple approaches to crystallizing polypeptides, no single set of conditions provides a reasonable expectation of success, especially when the crystals must be suitable for x-ray diffraction studies. Significant effort is required to obtain crystals of sufficient size and resolution to provide accurate information regarding the structure. For example, once a protein of sufficient purity is obtained, it must be crystallized to a size and clarity that is useful for x-ray diffraction and subsequent structure resolution. Further, although the amino acid sequence of a target protein may be known, this sequence information does not allow an accurate prediction of the crystal structure of the protein. Nor does the sequence information afford a complete understanding of the structural, conformational, and chemical interactions between a protein and a binding partner with which it interacts, such as IRF-4 and BATF. Thus, although crystal structures can provide a wealth of valuable information in the field of drug design and discovery, crystals of certain biologically relevant compounds and protein complexes such as IRF-4/AP-1 family members, such as BATF, are not readily available to those skilled in the art. High-quality, diffracting crystalline forms of IRF-4/AP-1 family members, such as BATF, assist the determination of its three-dimensional structure, which in turn is important for further understanding its biological role and for designing IRF-4/AP-1 family members, such as BATF, inhibitors, including, but not limited to, small molecule inhibitors.

In addition to providing structural information, crystalline polypeptides provide other advantages. For example, the crystallization process itself further purifies the polypeptide and satisfies one of the classical criteria for homogeneity. In fact, crystallization frequently provides unparalleled purification quality, removing impurities that are not removed by other purification methods such as HPLC, dialysis, conventional column chromatography, etc. Moreover, crystalline polypeptides are often stable at ambient temperatures and free of protease contamination and other degradation associated with solution storage. Crystalline polypeptides may also be useful as pharmaceutical preparations. Finally, crystallization techniques in general are largely free of problems such as denaturation associated with other stabilization methods (e.g. lyophilization).

IRF-4/AP-1 Interaction

The present invention is based in part on the discovery that IRF4 and AP-1, e.g., BATF, cooperatively bind to a representative set of target sequences containing novel IRF/AP-1 composite motifs. In one aspect, the present invention provides an isolated molecular complex comprising IRF-4 and an AP-1 family member. In one embodiment, the AP-1 family member is BATF. In another embodiment, the molecular complex further comprises a DNA sequence comprising an AP-1/IRF composite motif. In certain aspects, the present invention provides an isolated molecular complex consisting essentially of IRF-4, an AP-1 family member, and a DNA sequence consisting essentially of an AP-1/IRF composite motif.

As used herein, the term “AP-1/IRF composite motif” is intended to refer to a heterogeneous DNA sequence which allows co-binding of AP-1 and IRF. In one embodiment, the AP-1/IRF composite motif is a DNA sequence derived from a gene targeted by the IRF-4 and AP-1 complex. In another embodiment, the AP-1/IRF composite motif comprises an IRF site and an AP-1 site. In certain embodiments, there is no space between the IRF and AP-1 sites. In other embodiments, the AP-1/IRF composite motif comprises a 1 bp space between the IRF and AP-1 sites. In yet other embodiments, the AP-1/IRF composite motif comprises a 2 bp space between the IRF and AP-1 sites. In still other embodiments, the AP-1/IRF composite motif comprises a 3 bp space between the IRF and AP-1 sites. In certain preferred embodiments, the AP-1/IRF composite motif comprises a 4 bp space between the IRF and AP-1 sites. In a specific embodiment, the IRF site is TTTC. In another embodiment, the AP-1 site is TGACTCA. In yet another embodiment, the AP-1 site is TGAGTCA.

IRF-4/AP-1 Modulators

As shown herein, IRF4 and BATF function coordinately to induce expression of several functionally important T helper cell genes and the differentiation of Th17 cells. Accordingly, modulation of the interaction between IRF-4 and AP-1, e.g., BATF, provides a means to modulate Th17 mediated disease.

In some embodiments of the aspects described herein, an IRF-4/AP1 modulating agent is a nucleic acid, a nucleic acid analogue, protein, antibody, peptide, aptamer, oligomer of nucleic acids, amino acid, or carbohydrate, and includes, without limitation, proteins, oligonucleotides, ribozymes, DNAzymes, glycoproteins, siRNAs, lipoproteins, aptamers, and modifications and combinations thereof. IRF-4/AP1 agonists, activators, inhibitors, or antagonists can be naturally occurring and as a group, comprises synthetic ligands, small chemical molecules, peptidomimetics, antibodies or antigen-binding fragments thereof, polypeptides (e.g., dominant-negative IRF-4/AP1 polypeptides), inhibitory RNA molecules (i.e., siRNA or antisense RNA), and the like. Such IRF-4/AP1 modulating agents can be selected from compounds known to have a desired activity and/or property, or can be selected from a library of diverse compounds by screening methods, as known to one of skill in the art and as described herein.

In general, a polypeptide mimetic (“peptidomimetic”) is a molecule that mimics the biological activity of a polypeptide, but that is not peptidic in chemical nature. While, in certain embodiments, a peptidomimetic is a molecule that contains no peptide bonds (that is, amide bonds between amino acids), the term peptidomimetic may include molecules that are not completely peptidic in character, such as pseudo-peptides, semi-peptides and peptoids. Examples of some peptidomimetics by the broader definition (e.g., where part of a polypeptide is replaced by a structure lacking peptide bonds) are described below. Whether completely or partially non-peptide in character, peptidomimetics according to this invention may provide a spatial arrangement of reactive chemical moieties that closely resembles the three-dimensional arrangement of active groups in a polypeptide. As a result of this similar active-site geometry, the peptidomimetic may exhibit biological effects that are similar to the biological activity of a polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that may be obviated with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure, shape or reactivity. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98, all incorporated herein by reference].

Specific examples of peptidomimetics are set forth below. These examples are illustrative and not limiting in terms of the other or additional modifications.

Peptides with a Reduced Isostere Pseudopeptide Bond

Proteases act on peptide bonds. Substitution of peptide bonds by pseudopeptide bonds may confer resistance to proteolysis or otherwise make a compound less labile. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al. (1993), Int. J. Polypeptide Protein Res. 41:181-184, incorporated herein by reference). Thus, the amino acid sequences of these compounds may be identical to the sequences of their parent L-amino acid polypeptides, except that one or more of the peptide bonds are replaced by an isostere pseudopeptide bond. Preferably the most N-terminal peptide bond is substituted, since such a substitution would confer resistance to proteolysis by exopeptidases acting on the N-terminus.

Peptides with a Retro-Inverso Pseudopeptide Bond

To confer resistance to proteolysis, peptide bonds may also be substituted by retro-inverso pseudopeptide bonds (Dalpozzo, et al. (1993), Int. J. Polypeptide Protein Res. 41:561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

### Peptoid Derivatives

Peptoid derivatives of polypeptides represent another form of modified polypeptides that retain the important structural determinants for biological activity, yet eliminate the peptide bonds, thereby conferring resistance to proteolysis (Simon, et al., 1992, Proc. Natl. Acad. Sci. USA, 89:9367-9371 and incorporated herein by reference). Peptoids are oligomers of N-substituted glycines. A number of N-alkyl groups have been described, each corresponding to the side chain of a natural amino acid.

Methods of Identifying Th-17 Modulators

The present invention is based at least in part on the discovery of a novel IRF-4/AP-1/DNA complex. Accordingly, a suitable assay for determining inhibitors of IRF-4/AP-1 interaction includes, for example, electromobility shift assay (EMSA), as described herein. In one embodiment, the present invention includes a method for identifying an agent characterized by the ability to inhibit IRF-4/AP-1 family member interaction comprising a) incubating a reaction mixture comprising a candidate agent to be screened for the ability to inhibit IRF-4/AP-1 family member interaction, and a mixture of IRF-4 or an active fragment thereof and AP-1 family member or an active fragment thereof for a period of time and under conditions sufficient for IRF-4/AP-1 family member interaction; and b) determining the extent of IRF-4/AP-1 family member interaction relative to an otherwise identical reaction mixture which does not include said candidate agent, wherein a decrease in the interaction relative to that of the otherwise identical reaction mixture is indicative of said candidate agent having the ability to inhibit IRF-4/AP-1 family member interaction. In certain embodiments, the agent is a small molecule. In certain other embodiments, the agent is a peptidomimetic. In a particular embodiment, the IRF-4/AP-1 interaction is detected by electromobility shift assay (EMSA). In certain embodiments, DNA probes may be added to the reaction mixture comprising the agent to be screened, IRF-4 or an active fragment thereof, and AP-1 family member or an active fragment thereof. In one embodiment, the DNA probes comprise IRF and AP-1 motifs with different spacing requirements, e.g., 0, 1, 2, 3, 4, 5, 6, 7, 8 or 9 bp. In certain embodiments, the DNA probes comprising IRF and AP-1 motifs with different spacing requirements are derived from intronic regions within the CTLA-4 and Bcl11b genes. In some embodiments, the IRF site is TTTC. In certain embodiments, the AP-1 site is TGACTCA. In certain other embodiments, the AP-1 site is TGAGTCA. In a particular embodiment, an IRF/AP1 inhibitor may be screened using the electromobility shift assay (EMSA) as described in Example 3 herein.

In another embodiment, the present invention includes a method for identifying an agent characterized by the ability to inhibit IRF-4/AP-1 family member interaction, said method comprising a) providing a library of agents to be screened for the ability to inhibit IRF-4/AP-1 family member interaction and forming a reaction mixture comprising an agent to be screened for the ability to inhibit IRF-4/AP-1 family member interaction, and a mixture of IRF-4 or an active fragment thereof and AP-1 family member or an active fragment thereof; b) incubating a reaction mixture comprising an agent to be screened for the ability to inhibit IRF-4/AP-1 family member interaction, and a mixture of IRF-4 or an active fragment thereof and AP-1 family member or an active fragment thereof for a period of time and under conditions sufficient for IRF-4/AP-1 family member interaction; c) determining the extent of IRF-4/AP-1 family member interaction following the incubation of step b) relative to an otherwise identical incubation mixture which does not include an agent to be screened for the ability to inhibit IRF-4/AP-1 family member interaction, wherein a decrease in the interaction to that of the otherwise identical incubation mixture being indicative of the agent of step b) being characterized by the ability to inhibit IRF-4/AP-1 family member interaction. In certain embodiments, the agent is a small molecule. In certain other embodiments, the agent is a peptidomimetic. In a particular embodiment, the IRF-4/AP-1 interaction is detected by electromobility shift assay (EMSA). In certain embodiments, DNA probes may be added to the reaction mixture comprising the agent to be screened, IRF-4 or an active fragment thereof, and AP-1 family member or an active fragment thereof. In one embodiment, the DNA probes comprise IRF and AP-1 motifs with different spacing requirements, e.g., 0, 1, 2, 3, 4, 5, 6, 7, 8 or 9 bp. In certain embodiments, the DNA probes comprising IRF and AP-1 motifs with different spacing requirements are derived from intronic regions within the CTLA-4 and Bcl11b genes. In some embodiments, the IRF site is TTTC. In certain embodiments, the AP-1 site is TGACTCA. In certain other embodiments, the AP-1 site is TGAGTCA. In a particular embodiment, an IRF/AP1 inhibitor may be screened using the electromobility shift assay (EMSA) as described in Example 3 herein.

Other binding assays, and methods of determining regulation of gene expression will be apparent to those of skill in the art. In one embodiment, a BIAcore machine can be used to determine the binding constant of a complex between protein and a candidate compound or between a protein and its binding partner or ligand, for example, in the presence and absence of the candidate compound. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Contacting a candidate compound at various concentrations with the protein and monitoring the response function (e.g., the change in the refractive index with respect to time) allows the complex dissociation constant to be determined in the presence of the candidate compound.

Other suitable assays for measuring the binding of a candidate compound to a protein, and/or for measuring the ability of such compound to affect the binding of protein to its binding partner or ligand include, for example, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desomtion/ionization time-of-flight (MALDI-TOF) mass spectrometry, microcytometry, microarray, microscopy, fluorescence activated cell sorting (FACS), and flow cytometry.

A variety of assay formats will suffice and, in light of the present disclosure, those not expressly described herein will nevertheless be understood by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as inhibitors or stimulators of IRF-4/AP-1 interaction, e.g., IRF-4/BATF, can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In a specific embodiment, the test agent is a small organic molecule having a molecular weight of less than about 2,000 daltons.

The test compounds of the invention can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase, photoactivatible crosslinkers or any combinations thereof.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an inhibition of IRF-4/AP-1, e.g., BATF, interaction.

Merely to illustrate, in an exemplary screening assay of the present invention, the compound of interest is contacted with an isolated and purified IRF-4 polypeptide which is ordinarily capable of binding to an AP-1 family member, e.g., BATF, as appropriate for the purpose of the assay. To the mixture of the compound and IRF-4 polypeptide is then added a composition containing an AP-1 family member, e.g., BATF. Detection and quantification of IRF-4/AP1 complexes provides a means for determining the compound's efficacy at inhibiting (or potentiating) complex formation between the IRF-4 and the AP-1 family member, e.g., BATF. The efficacy of the compound can be assessed by generating dose response curves from data obtained using various concentrations of the test compound. Moreover, a control assay can also be performed to provide a baseline for comparison. For example, in a control assay, isolated and purified IRF-4 polypeptide is added to a composition containing the AP-1 family member, and the formation of IRF-4/AP-1 complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between the IRF-4 and the AP-1 family member, e.g., BATF, may be detected by a variety of techniques. For instance, modulation of the formation of complexes can be quantitated using, for example, detectably labeled proteins such as radiolabelled (e.g., ³²P, ³⁵S, ¹⁴C or ³H), fluorescently labeled (e.g., FITC), or enzymatically labeled IRF-4 or AP-1, by immunoassay, or by chromatographic detection.

In certain embodiments, the present invention contemplates the use of fluorescence polarization assays and fluorescence resonance energy transfer (FRET) assays in measuring, either directly or indirectly, the degree of interaction between IRF-4 and AP-1 (e.g., BATF). Further, other modes of detection such as those based on optical waveguides (PCT Publication WO 96/26432 and U.S. Pat. No. 5,677,196), surface plasmon resonance (SPR) (the mode employed by BiaCore systems used in the Examples, below), surface charge sensors, and surface force sensors are compatible with many embodiments of the invention.

Moreover, the present invention contemplates the use of an interaction trap assay, also known as the “two hybrid assay,” for identifying agents that disrupt or potentiate interaction between IRF-4 and AP-1 (e.g., BATF). See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present invention contemplates the use of reverse two hybrid systems to identify compounds (e.g., small molecules or peptidomimetics) that dissociate interactions between IRF-4 and AP-1 (e.g., BATF). See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; 5,965,368.

In certain embodiments, the subject compounds are identified by their ability to interact with a IRF-4/AP-1 complex of the invention. The interaction between the compound and the IRF-4/AP-1 complex may be covalent or non-covalent. For example, such interaction can be identified at the protein level using in vitro biochemical methods, including photo-crosslinking, radiolabeled ligand binding, and affinity chromatography (Jakoby W B et al., 1974, Methods in Enzymology 46: 1). In certain cases, the compounds may be screened in a mechanism based assay, such as an assay to detect compounds which bind to a IRF-4/AP-1 complex. This may include a solid phase or fluid phase binding event. Alternatively, the genes encoding IRF-4 and AP-1, e.g., BATF, can be transfected with a reporter system (e.g., β-galactosidase, luciferase, or green fluorescent protein) into a cell and screened against the library preferably by a high throughput screening or with individual members of the library. Other mechanism based binding assays may be used, for example, binding assays which detect changes in free energy. Binding assays can be performed with the target fixed to a well, bead or chip or captured by an immobilized antibody or resolved by capillary electrophoresis. The bound compounds may be detected usually using colorimetric or fluorescence or surface plasmon resonance.

In certain aspects, the present invention provides methods and agents for modulating Th-17 mediated diseases. Therefore, any compound identified using a cell-free system, or any other compound that is expected to affect IRF-4/AP-1 interaction, can be tested in whole cells or tissues, in vitro or in vivo, to confirm their ability to modulate Th-17 mediated diseases. Various methods known in the art can be utilized for this purpose. Further, these screening assays are useful for drug target verification and quality control purposes.

Agents suitable for the treatment of Th17-mediated diseases as described herein can further be selected using well known animal models, non-limiting examples of which are provided herein.

Experimental autoimmune encephalomyelitis (EAE) is a T cell mediated autoimmune disease characterized by T cell and mononuclear cell inflammation and subsequent demyelination of axons in the central nervous system. EAE is generally considered to be a relevant animal model for multiple sclerosis (MS) in humans. Bolton, C., Multiple Sclerosis (1995) 1:143. Both acute and relapsing-remitting models have been developed.

Animal models of psoriasis have also been developed. Thus, Asebia (ab), flaky skin (fsn), and chronic proliferative dermatitis (cpd) are spontaneous mouse mutations with psoriasis-like skin alterations. Transgenic mice with cutaneous overexpression of cytokines, such as interferon-γ, interleukin-1α, keratinocyte growth factor, transforming growth factor-α, interferon-6, vascular endothelial growth factor, or bone morphogenic protein-6, can also be used to study in vivo psoriasis and to identify therapeutics for the treatment of psoriasis. Psoriasis-like lesions were also described in β₂-integrin hypomorphic mice backcrossed to the PL/J strain and in βB₁-integrin transgenic mice, scid/scid mice reconstituted with CD4⁺/CD45RB^{hi}T lymphocytes as well as in HLA-B27/hβ₂m transgenic rats. Xenotransplantation models using human skin grafted on to immunodeficient mice are also known. Thus, the compounds of the invention can be tested in the scid/scid mouse model described by Schon, M. P. et al, Nat. Med. (1997) 3:183, in which the mice demonstrate histopathologic skin lesions resembling psoriasis. Another suitable model is the human skin/scid mouse chimera prepared as described by Nickoloff, B. J. et al, Am. J. Path. (1995) 146:580. For further details see, e.g. Schon, M. P., J Invest Dermatology 112:405-410 (1999).

Modulating Th17-Mediated Diseases

Certain aspects of the methods described herein are based, in part, on the discovery that IRF-4/AP-1 function coordinately to induce the expression of several important T helper genes, e.g., CTLA-4 and ICOS, and in the differentiation of Th17 cells. Thus, in some aspects described herein are methods of identifying inhibitors of IRF-4/AP-1 interaction, and inhibiting Th17-mediated immune responses. In other aspects described herein are methods for or promoting or increasing Th17 differentiation and activity, and promoting and increasing Th17-mediated immune responses. Accordingly, the methods using the IRF-4/AP-1 inhibitors, e.g., IRF-4/BATF, described herein are useful in the treatment of subjects having diseases or disorders mediated or modulated by Th17 expression and or activity, such as autoimmunity, chronic inflammatory disorders, infectious diseases, cancer, allergic conditions, and the like. Due to the complexity of Th-17 mediated diseases and related conditions, such as, for example, autoimmunity, chronic inflammatory disorders, depending on the stage and nature of the disease or condition and the timing of administration, in certain embodiments, IRF-4/AP-1 stimulators may also be useful in the methods of the present invention.

A “Th17-mediated immune response” refers to an immune response that is associated with the induction of, differentiation of, expansion of, proliferation of, functional activity of, or a combination thereof, one or more Th17 cells. At a minimum, as used herein, a “Th17 cell” refers to a CD4+ T cell that expresses and/or produces IL-17A, also known herein as “IL-17.” In some embodiments, a Th17 cell is further characterized by expression of one or more cytokines selected from the following: IL-17F, IL-22, IL-26, IL-21, and TNF-OC. In some embodiments, a Th17 cell is further characterized by cell-surface expression of the chemokine receptor CCR6. In some embodiments, a Th17 cell is further characterized by cell-surface expression of the chemokine receptors CCR6 and CCR4. In some embodiments, a Th17 cell is further characterized by cell-surface expression of the chemokine receptor CCR6 and IL23R. In some embodiments, a Th17 cell is further characterized by cell-surface expression of the C-type lectin CD161. In some embodiments, a Th17 cell can be further characterized by expression or activity of one or more of the following factors: RORγt, RORα, STAT3, IRF4, the AhR (aryl hydrocarbon receptor), and BATF. In some embodiments, a Th17 cell can be further characterized as a cell expressing or producing IL-17, but not expressing or producing certain cytokines, such as IL-4, IL-5, and IFN-γ. In some embodiments, a Th17 cell can be further characterized as a cell expressing or producing IL-17, but not expressing or producing certain transcription factors such as T-bet, GAT-A-3, FOXP3, STAT1, STAT4, and STAT5.

Th 17 cells as described herein can be generated or propagated under a variety of conditions. In certain embodiments, a Th 17 cell can be generated or derived from a naïve CD4⁺, CD62L⁺, CD25⁻ T cells in Th17 polarizing conditions. In some embodiments, a Th 17 cell can be generated or derived from a naïve CD4⁺ T cell in the presence of TGF-β and IL-6. In some embodiments, a Th17 cell can be generated or derived from a naïve CD4⁺ T cell in the presence of TGF-β and IL-21. In other embodiments, a Th17 cell or a population of Th 17 cells is generated or derived from expansion of a population of Th 17 cells in the presence of IL-23. In other embodiments, a population of Th 17 cells can be maintained in the presence of IL-23.

Pharmaceutical Compositions

An IRF-4/AP1 modulator, such as the IRF-4/AP1 inhibitors, e.g., small molecule or peptidomimetic IRF-4/AP1 inhibitors, RNA-based IRF-4/AP1 inhibitors, and blocking anti-IRF-4/AP1 antibodies or antigen-binding fragments described herein, and the IRF-4/AP1 activators, such as activating anti-IRF-4/AP1 antibodies and antigen-binding fragments thereof, can be formulated, dosed, and administered in a fashion consistent with good medical practice for use in the treatment of the Th17-mediated disorders described herein, such as autoimmune disorders.

Factors for consideration in this context include the particular disorder or type of disorder being treated, the particular subject being treated, the clinical condition of the individual subject, the cause of the disorder, the site of delivery of the agent, the method of administration, the scheduling of administration, and other factors known to medical practitioners.

Accordingly, the “therapeutically effective amount” of an IRF-4/AP-1 modulator, such as the IRF-4/AP1 inhibitors, e.g., small molecule or peptidomimetic IRF-4/AP1 inhibitors, and the IRF-4/AP1 activators described herein, to be administered is governed by such considerations, and, as used herein, refers to the minimum amount necessary to prevent, ameliorate, or treat, or stabilize, the Th17-mediated disorder. In some embodiments, an IRF-4/AP-1 modulator, is optionally formulated with one or more agents currently used to prevent or treat the disorder being treated. The effective amount of such other agents depends on the amount of the IRF-4/AP-1 modulator present in the formulation, the type of disorder or treatment, and other factors discussed herein, and as understood by one of skill in the art. These are generally used in the same dosages and with administration routes as used herein above or from about 1 to 99% of the heretofore employed dosages.

An effective amount as used herein also includes an amount sufficient to delay the development of a symptom of the Th17-mediated disorder, alter the course of the Th17-mediated disorder (for example but not limited to, inhibit or delay time until relapse in relapsing-remitting multiple sclerosis), or reverse a symptom of the autoimmune disease or disorder. Thus, it is not possible to specify an exact “effective amount”. However, for any given case, an appropriate “effective amount” can be determined by one of ordinary skill in the art using only routine experimentation. If a certain amount of an IRF-4/AP-1 modulator as described herein statistically significantly alters an indicium of a Th17 response, e.g., decreases the number of Th17 cells, reduces the production of IL-17, reduces the proliferation of Th17 cells, and/or reduces trafficking of Th17 cells, as defined herein, it is evidence that said amount is therapeutically effective.

Accordingly, as used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a chronic immune condition, such as, but not limited to, an autoimmune disorder, a chronic inflammatory disorder, an infection, or a cancer.

Treatment is generally “effective” if one or more symptoms, clinical markers, or indicia of disease are reduced to a clinically significant degree. Alternatively, treatment is “effective” if the progression of a disease is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

For example, in some embodiments, the methods described herein comprise administering an effective amount of the IRF-4/AP-1 inhibitors described herein to a subject in order to alleviate one or more symptoms of an autoimmune disorder. As used herein, “alleviating a symptom of an autoimmune disorder” refers to ameliorating any condition or symptom associated with the autoimmune disorder. Alternatively, alleviating a symptom of an autoimmune disorder can involve reducing the number of autoimmune cells in the subject relative to the number of autoimmune cells in an untreated control. In some embodiments, the autoimmune cells comprise Th17 cells. As compared with an equivalent untreated control, such reduction or degree of prevention is at least 5%, 10%, 20%, 40%, 50%, 60%, 80%, 90%, 95%, or 100% as measured by any standard technique. Desirably, the autoimmune disorder is completely abrogated, as detected by any standard method known in the art, in which case the autoimmune disorder is considered to have been cured. A patient who is being treated for an autoimmune disorder is one who a medical practitioner has diagnosed as having such a condition. Diagnosis can be by any suitable means. Diagnosis and monitoring can involve, for example, detecting the level of autoimmune cells or autoantibodies in a biological sample (for example, a tissue biopsy, blood or serum test, or urine test), detecting the level of a surrogate marker of the autoimmune disorder in a biological sample, detecting symptoms associated with the autoimmune disorder, or detecting immune cells involved in the immune response typical of the autoimmune disorder (for example, detection of self-antigen-specific T cells that secrete inflammatory cytokines, such as IL 17).

In other embodiments, the methods described herein comprise administering an effective amount of the IRF-4/AP-1 inhibitors or IRF-4/AP-1 activators described herein to a subject in order to alleviate one or more symptoms of a cancer or tumor in a subject in need thereof. As used herein, “alleviating a symptom of a cancer” refers to ameliorating any condition or symptom associated with the cancer. In preferred embodiments, an IRF-4/AP-1 modulator described herein can produce marked anticancer effects in a human subject without causing significant toxicities or adverse effects. The efficacy of the IRF-4/AP-1 treatments described herein can be measured by various parameters commonly used in evaluating cancer treatments, including but not limited to, tumor regression, tumor weight or size shrinkage, reduction in rate of tumor growth, the presence or the size of a dormant tumor, the presence or size of metastases or micrometastases, degree of tumor or cancer invasiveness, size or number of the blood vessels, time to progression, duration of survival, progression free survival, overall response rate, duration of response, and quality of life.

Effective amounts, toxicity, and therapeutic efficacy of the IRF-4/AP-1 modulators, such as the IRF-4/AP1 inhibitors, e.g., small molecule or peptidomimetic IRF-4/AP1 inhibitors described herein, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of the IRF-4/AP-1 inhibitor or IRF-4/AP-1 activator), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

Depending on the type and severity of the disease, about 1 μg/kg to 100 mg/kg (e.g., 0.1-20 mg/kg) of e.g., a small molecule IRF-4/AP-1 inhibitor identified by the methods described herein, is an initial candidate dosage range for administration to the subject, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until the cancer is treated, as measured by the methods described above or known in the art.

However, other dosage regimens may be useful. The progress of the therapeutic methods described herein is easily monitored by conventional techniques and assays, such as those described herein, or known to one of skill in the art. In other embodiments, such dosing regimen is used in combination with a chemotherapy regimen as the first line therapy for treating locally recurrent or metastatic breast cancer.

The duration of the therapeutic methods described herein can continue for as long as medically indicated or until a desired therapeutic effect (e.g., those described herein) is achieved. In certain embodiments, administration of an IRF-4/AP-1 modulator, i.e., “IRF-4/AP-1 inhibitor therapy” or “IRF-4/AP-1 activator therapy” is continued for at least 1 month, at least 2 months, at least 4 months, at least 6 months, at least 8 months, at least 10 months, at least 1 year, at least 2 years, at least 3 years, at least 4 years, at least 5 years, at least 10 years, at least 20 years, or for at least a period of years up to the lifetime of the subject.

The IRF-4/AP-1 modulators described herein, such as the IRF-4/AP1 modulator, such as the IRF-4/AP1 inhibitors, e.g., small molecule or peptidomimetic IRF-4/AP1 inhibitors, RNA-based IRF-4/AP1 inhibitors, and blocking anti-IRF-4/AP1 antibodies or antigen-binding fragments described herein, and the IRF-4/AP1 activators, such as activating anti-IRF-4/AP1 antibodies and antigen-binding fragments thereof, can be administered to a subject, e.g., a human subject, in accordance with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Local administration can be used if for example, extensive side effects or toxicity is associated with the IRF-4/AP-1 inhibitor or the IRF-4/AP-1 activator. An ex vivo strategy can also be used for therapeutic applications.

Exemplary modes of administration of the IRF-4/AP-1 modulators described herein, such as the IRF-4/AP1 inhibitors, e.g., small molecule or peptidomimetic IRF-4/AP1 inhibitors, RNA-based IRF-4/AP1 inhibitors, and blocking anti-IRF-4/AP1 antibodies or antigen-binding fragments described herein, and the IRF-4/AP1 activators, such as activating anti-IRF-4/AP1 antibodies and antigen-binding fragments thereof, include, but are not limited to, injection, infusion, inhalation (e.g., intranasal or intratracheal), ingestion, rectal, and topical (including buccal and sublingual) administration. The phrases “parenteral administration” and “administered parenterally” as used herein, refer to modes of administration other than enteral and topical administration, usually by injection. As used herein, “injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein refer to the administration of an IRF-4/AP-1 modulator, such as the IRF-4/AP1 inhibitors, e.g., small molecule or peptidomimetic IRF-4/AP1 inhibitors, and the IRF-4/AP-1 activators described herein, other than directly into a target site, tissue, or organ, such as the lung, such that it enters the subject's circulatory system and, thus, is subject to metabolism and other like processes.

In some embodiments, the IRF-4/AP-1 modulators, such as the IRF-4/AP1 inhibitors, e.g., small molecule or peptidomimetic IRF-4/AP1 inhibitors, and the IRF-4/AP-1 activators described herein, are administered by intravenous infusion or injection. In some embodiments, where local treatment is desired, for example, at or near a site of a Th17-mediated immune response, such as in a joint of a patient having rheumatoid arthritis, the IRF-4/AP-1 modulators, such as the IRF-4/AP1 inhibitors, e.g., small molecule or peptidomimetic IRF-4/AP1 inhibitors, and the IRF-4/AP-1 activators can be administered by intralesional administration. Additionally, in some embodiments, the IRF-4/AP-1 inhibitors or IRF-4/AP-1 activators described herein can be administered by pulse infusion, particularly with declining doses of the inhibitors or non-constitutive agonists. Preferably the dosing is given by injections, most preferably intravenous or subcutaneous injections, depending in part on whether the administration is brief or chronic.

In another embodiment of the invention, an article of manufacture is provided which contains the formulation and preferably provides instructions for its use. The article of manufacture comprises a container. Suitable containers include, for example, bottles, vials (e.g., dual chamber vials), syringes (such as single or dual chamber syringes) and test tubes. The container may be formed from a variety of materials such as glass or plastic. The label, which is on, or associated with, the container holding the formulation may indicate directions for reconstitution and/or use. The label may further indicate that the formulation is useful or intended for subcutaneous administration. The container holding the formulation may be a multi-use vial, which allows for repeat administrations (e.g., from 2-6 administrations) of the reconstituted formulation. The article of manufacture may further comprise a second container comprising a suitable diluent (e.g., BWFI). Upon mixing of the diluent and the lyophilized formulation, for example, the final protein concentration in the reconstituted formulation will generally be at least 50 mg/ml. The article of manufacture may further include other materials desirable from a commercial and user standpoint, including other buffers, diluents, filters, needles, syringes, and package inserts with instructions for use.

The following examples are offered for illustrative purposes only, and are not intended to limit the scope of the present invention in any way.

All patent and literature references cited in the present specification are hereby expressly incorporated by reference in their entirety.

### EXAMPLES

### Materials and Methods

### CD4 T Cell Isolation and Differentiation

Mice were housed in a specific pathogen-free barrier facility. Naïve CD4⁺CD62L⁺CD25″ T cells were isolated by cell sorting from spleens of 6-12 week old C57BL/6 mice of the following genotypes wild type, Irf4+/fl, CD4Cre or Irf4fl/fl, CD4Cre mice (U. Klein et al., Nature Immunology 7, 773 (2006); P. P. Lee et al., Immunity 15, 763 (2001)) and activated with plate-bound anti-CD3 (BD Bioscience, 145-2C11, 5 mg/ml) and soluble anti-CD28 (BD Bioscience, 37.51, 2.5 mg/ml). Cultures were supplemented with anti-IL-4 (BD Bioscience, 11B11, 10 μg/ml), anti-IFNγ (BD Bioscience, XMG1.2, 10 μg/ml) rmIL-6 (R&D Systems, 20 ng/ml), rmIL-23 (R&D Systems, 20 ng/ml) and rhTGF-β (R&D Systems, 1 ng/ml) for Th17 differentiation. For Th0 differentiation, cultures were supplemented with anti-IL-4 and anti-IFNγ as described above in addition to anti-IL-12 (BD Bioscience, C17.8, 10 μg/ml). Medium for Th2 differentiating conditions contained anti-IFNγ and anti-IL-12 as described above in addition to rmIL-4 (R7D Systems, 10 ng/ml).

### Retroviral Transduction of CD4 T Cells

293T cells were used to generate retroviral stocks by transfecting pMSCV-IRES-GFP or pMSCV-IRES-hCD4 or their counterparts encoding IRF-4 and BATF, respectively. Retroviral supernatants were collected 3 days after transfection. Naïve CD4⁺CD62L⁺ T cells were isolated from the spleens of 6-12 week old C57BL/6 mice (Jackson Labs) using MACS microbeads (Miltenyi, #130-093-227). Cells were activated under either non-polarizing or Th17 inducing conditions as described above with slight modifications (A. Brustle et al., Nature Immunology 8, 958 (2007)). T cells (Th0 or Th17) were spin-infected with retroviral supernatants 42 hours after their activation. GFP and hCD4 (anti-hCD4-APC, BD, RPA-T4) were used as FACS markers to analyze infected cells. After stimulation of cells with PMA and ionomycin for four hours (A. Brustle et al., Nature Immunology 8, 958 (2007)), FACS was used to monitor intracellular CTLA-4 and IL-17A expression (anti-IL-17A-PE, BD, 12-7177-81 and anti-CTLA-4-PE, BD, 12-1522-81).

### Generation and LPS Stimulation of Dendritic Cells

Bone marrow was flushed from femur/tibias of C57BL/6 mice and RBCs were lysed in red cell lysis buffer. B and T cells were removed with the following antibodies: CD5 (BD, 53-7.3), B220 (BD, RA3-6B2), CD19 (Biolegend, 6D5) CD4 (BD, RM4-5), CD8a (BD, 53-6.7) and anti-Biotin beads (Miltenyi #130-090-485). Bone marrow derived hematopoietic progenitors were cultured for 5 days in RPMI supplemented with mGMCSF (Peprotech, 10 ng/ml) and mIL-4 (Peprotech, 5 ng/ml) to generate dendritic cells. These cells were stimulated for 6 hours with 100 ng/ml LPS (Sigma) and CD11c+ cells were purified with MACS microbeads (Miltenyi #130-052-001) for ChIPseq analysis.

### Immunoblotting

Proteins from whole-cell lysates were resolved using SDS-PAGE and transferred to a PVDF membrane. The blots were probed with anti-IRF-4 (Santa Cruz, M-17), anti-BATF or anti-α-Actin (Sigma, A5441) antibodies and visualized with appropriate HRP-coupled secondary antibodies. BATF antibodies were generated by immunizing rabbits with a mixture of two peptides (aa 1-22 and aa 108-125) followed by affinity purification.

### ChIP and ChIPseq Analyses

ChIP assays were performed as previously described (R. Sciammas et al., Immunity 25, 225 (2006)). Chromatin from Th0, Th2 or Th17 cells (2×107) or CD11c⁺ DC cells (7.5×106) was sonicated for 25 min using Covaris E210 (20% duty cycle, intensity 5, 200 cycles per burst) to obtain DNA fragments ranging in size from 100 to 500 bp. Chromatin fragments were immunoprecipitated using the IRF-4 or BATF antibodies detailed above. Specific DNA sequences were assessed by quantitative real-time PCR after reversal of formaldehyde crosslinks. Primers used for PCR are listed in Table 1 below. Genomic DNA was sequenced using Illumina Genome Analyzer II, and aligned using GSNAP (Genomic Short-read Nucleotide Alignment Program) (T. D. Wu, et al., Bioinformatics 26, 873 (2010)).

TABLE 1
Chip primers (SEQ ID NOS 5-20, respectively,
in order of appearance).

| Primer name | Sequence |
|---|---|
| Ctla4 Forw | TGTTTGTGTGCTTCTGAGCAGGGT |
| Ctla4 Rev | GGGTTGCCCAGAGACTGCTGTGT |
| IL17a Int 1 Forw | ACATGAGTGCCGACAAACAACGGGT |
| IL17A Int 1 Rev | AGTCAGTGGGCTGCTGAATGACCCC |
| IL21 Pro Forw | GCACAGGAGGCACCGTCAGATTT |
| IL21 Pro Rev | GGCCATGCCGCTGCTTTACTCA |
| IL21 Int 2 Forw | CCAACCTGTGCACTTGGCCACC |
| IL21 Int 2 Rev | GGAAGGCCGGCACTTTGCCCT |
| IL23R Int 3 Forw | GGAAGGAAGTTTGCCAAGAACCCAG |
| IL23R Int 3 Rev | TTATGAGTGTGCAAACCCAACTGCC |
| IL12rb1 Pro Forw | GCAGAGGGGCCCCCACCTTT |
| IL12rb1 Pro Rev | CCGCCTTGTCCACGCTCAGA |
| IL23R Pro Forw | GCATTTGCTTCAGGCTTTTACCTATG |
| IL23R Pro Rev | GGCAGCTCACTTTCAGTAATCTGGG |
| Rorc control Forw | CAGGGCCAGGGTCTTCGGGT |
| Rorc control Rev | AGCAGGTGCTGGGTAAGTGTCAG |

### Computational Methods for ChIPseq

IRF-4 and BATF binding peaks were identified using quantitative enrichment of Sequence Tags (QuEST) v2.4 with input chromatin as control, setting a 30-fold enrichment threshold. The MEME algorithm (v4.8.1) was used to identify motifs within 100 bp (+/−) of the peak maxima. Genomic location and distance of binding sites from the nearest transcriptional start site (TSS) were analyzed with the ChlPpeakAnno bioconductor package. Peaks were annotated with Refseq genes; a gene was associated with a peak if its TSS was within 50 kb of the peak or if it was the nearest gene to the peak. Peaks were called as coincident if their maxima were less than 100 bp apart.

### Transient Transfection of 293T Cells

CMV-based vectors (pcDNA3 or pRK5) encoding murine IRF-4, IRF-8, IRF-3, BATF, JunB, c-Jun or FosL2 were used to enable transient expression of individual proteins and preparation of nuclear extracts. 293T cells (ATCC) were transfected with Lipofectamine 2000 (Invitrogen) or FuGene HD (Roche) according to the manufacturer's protocols.

### Electromobility Shift Assays (EMSAs)

Nuclear extracts were prepared from 293T cells (2×107), 48 hours after their transfection or from Th17 cells (8×107), 42 hours after their activation and differentiation. Cells were incubated for 10 min in 1 ml of hypotonic buffer (10 mM Hepes pH7.6, 10 mM KCl, 0.1 mM EGTA, 1.5 mM MgCl, 1 mM DTT, 0.5 mM PMSF and the complete protease inhibitor mixture (Roche, #11697498001) and then lysed with the addition of 0.01% Triton-x-100. After centrifugation at maximum speed in an Eppendorf microfuge for 15 seconds the nuclear pellet was resuspended in 80 μl of the following buffer (420 mM NaCl, 20 mM Hepes pH7.9, 0.2 mM EDTA, 25% Glycerol, 1 mM DTT, 0.5 mM PMSF with complete protease inhibitor mixture). Nuclear proteins were extracted with intermittent vortexing and incubation for 15 minutes at 40 C. In-vitro binding assays were performed using reagents from the light-shift chemiluminescent EMSA kit (Thermo scientific) according to the manufacturer's protocol, with the exception of using FAMlabeled probes (Table 2, below). Protein-DNA complexes were resolved using 6%-TBE Gels (Invitrogen) at 100V and imaged with a Typhoon Trio Imager (Amersham Biosciences). Following antibodies were used for supershifting of protein-DNA complexes; IRF-4 (Santa Cruz, M-17), BATF (Sigma, 8A12), JunB (Santa Cruz, C11), c-Jun (Santa Cruz, sc-45X), IRF-8 (Santa Cruz, C-19X), IRF-3 (Santa Cruz, C-20X) and pan-Fos (Santa Cruz, K-25) and rabbit IgG (Santa Cruz, sc-2027).

TABLE 2
EMSA probes (SEQ ID NOS 21-49, respectively,
in order of appearance).

| Probe | Sequence |
|---|---|
| Bcl11b | TAGTGCAGAAATGAGTCAGA |
| | GATCAAAGAAG |
| Bcl11b IRFmut | TAGTGCAtAcATGAGTCAGA |
| | GATCAAAGAAG |
| Bcl11b AP-1mut | TAGTGCAGAAAgGcGTCAGA |
| | GATCAAAGAAG |
| Bcl11b IRFAP-1mut | TAGTGCATAcAgGcGTCAGA |
| | GATCAAAGAAG |
| CTLA4 | CTTGCCTTAGAGGTTTCGGG |
| | ATGACTAATACTGTACGTGA |
| CTLA4 IRFmut | CTTGCCTTAGAGGT [symbol] TaGGG |
| | ATGACTAATACTGTACGTGA |
| CTLA AP-1mut | CTTGCCTTAGAGGTTTCGGG |
| | AgGcCTAATACTGTACGTGA |
| CTLA4 IRFAP-1mut | CTTGCCTTAGAGGTgTaGGG |
| | AgGcCTAATACTGTACGTGA |
| Il12rb1 Pro | GCTTTTGCTTTCACTTTGAC |
| | TGGCCTGGAGACAATGAGTT |
| Il12rb1 Pro IRFmut | GCTTTTGCTgTaACTTTGAC |
| | TGGCCTGGAGACAATGAGTT |
| Il12rb1 Pro AP 1mut | GCTTTTGCTTTCACTTgGcC |
| | TGGCCTGGAGACAATGAGTT |
| IL21 Pro | AAA [symbol] [symbol] CACTTCTGCAATGA |
| | GTAAACCACCCCCATCCCCTT |
| IL21 Pro IRfleftmut | AAAGGCACTTCTtCAtTGAG |
| | TAAAGCAGCGGCATGGCCTT |
| IL21 Pro [symbol] AP-1mut | AAAGGCACTTCTGCAAgGcG |
| | TAAAGCAGCGGCATGGCCTT |
| IL23R Pro | CGTGTCCATTTCATAATGAC |
| | TAGTTGTCATGTCACTATTT |
| IL23R Pro IRFmut | CGTGTCCATgTaATAATGAC |
| | TAGTTGTCATGTCACTATTT |
| IL23R Pro AP-1mut | CGTGTCCATTTCATAAgGcC |
| | TAGTTGTCATGTCACTATTT |
| IL17A Int 1 | AGGTTTACAGAAGGAGTGAC |
| | TAAGAACACTAGTGCATAAG |
| IL17A Int 1 IRFmut | AGGTTTACAGAAGGAGTGAC |
| | TAAtcATACTAGTGCATAAG |
| IL17A Int 1 AP-1mut | AGGTTTACAGAAGGAGTGAC |
| | [symbol] AcGAACACTAGTGCATAAG |
| IL21 Prom | TGGCCACCGTGAATGTGACT |
| | AATAACAGGAGCCCATCAG |
| IL21 Pro IRFmut | TGGCCACCGTGAATGTTGAC |
| | TAATcAtAGGAGCCCATCAG |
| IL21 Pro AP-1mut | TGGCCACCGTGAATGTgGAc |
| | TAATAACAGGAGCCCATCAG |
| IL23R Int 3 | CTTGAACCCAGGAGTTTGAG |
| | TAAATTCTTGCCTGGCAGTT |
| IL23R Int 3 IRFmut | CTTCAACCCACCACTTTCAG |
| | TAAtcatTTCCCTCCCACTT |
| IL23R Int 3 AP-1mut | CTTCAACCCACCACTTgGcG |
| | TAAATTCTTCCCTCCCACTT |
| Il21 Int 2 | TGGCCACCGTGAATGTTGAC |
| | TAATAACAGGAGCCCATCAG |
| Il21 Int 2 IRFmut | TGGCCACCGTGAATGTTGAC |
| | TAATcAtAGGAGCCCATCAG |
| Il21 Int 2 AP-1mut | TGGCCACCGTGAATGTgGAc |
| | TAATAACAGGAGCCCATCAG |

[symbol]

indicates data missing or illegible when filed

### RNA Analysis (RT-PCR and Microarray)

Total RNA was isolated from in-vitro differentiated Th17 cells using RNeasy Plus Minikit (Qiagen, #74134) and cDNA was prepared with SuperScript II reverse transcriptase kit (Invitrogen, #11754-050). Quantitative PCR was performed using a SYBR qPCR Master Mix (Agilent, #600828-51). Primers used are listed in Table 3 below. Microarray analysis of Irf4^{+/-} and Irf4^{-/-} CD4+ T-cells differentiated under Th17 polarizing conditions was performed in triplicate with the Agilent WMG 4×44K arrays. The data was analyzed with the Partek Genomics Suite software. Genes differentially expressed by at least 2-fold with p<0.05 were considered to be significantly regulated by IRF-4. Network analysis of IRF-4 regulated and IRF-4/BATF co-targeted genes was performed using Ingenuity Pathway Analysis (IPA). Network diagram of biologically relevant transcriptional modules discovered in the IRF-4 regulated and IRF-4/BATF targeted genes was created using BioTapestry.

TABLE 3
RT-PCR primers (SEQ ID NOS 50-67, respectively,
in order of appearance).

| Primer name | Sequence |
|---|---|
| Il17a Forw | CAGCGTGTCCAAACACTGAGGCCA |
| Il17a Rev | CATTGCGGTGGAGAGTCCAGGGTG |
| Ctla4 Forw | ACCCACCGCCATACTTTGTGGGC |
| Ctla4 Rev | TCCGGGCATGGTTCTGGATCAATGA |
| Hprt Forw | GCAGTACAGCCCCAAAATGG |
| Hprt Rev | AACAAAGTCTGGCCTGTATCCAA |
| Irf4 Forw | GTGCGGGTGAGCGCACAAGC |
| Irf4 Rev | CAGCCCGGGGTACTTGCCGC |
| Il21 Forw | AGCCCCCAAGGGCCAGATCGC |
| Il21 Rev | AGCTGCATGCTCACAGTGCCCCTTT |
| Il22 Forw | GCTCCTGTCACATCAGCGGTGAC |
| Il22 Rev | GCAGGTCCAGTTCCCCAATCGCC |
| IL23R Forw | CCCAGACAGTTTCCCAGGTTACAGC |
| IL23R Rev | TGGCCAAGAAGACCATTCCCGACA |
| IL12rb1 Forw | CGAGCCGACACTGCGAGGCT |
| IL12rb1 Rev | AAGTGCCAGGCGGCCCTGTTT |
| CCR3 Forw | AATATGAGTGGGCACCACCCTGTGA |
| CCR3 Rev | ACACAACCATCATGTTGCCCAGGAG |

### Protein-DNA Modeling

The models of the complexes of IRF-4/BATF/JunB bound to the Bcl11b and CTLA-4 DNA sites were made using the programs Pymol (PyMOL, L. Schrodinger, Ed.), Modeller (N. Eswar, et al., Current Protocols in Bioinformatics. (John Wiley and Sons, Inc., 2006) LSQMAN (G. F. Kleywegt, Acta Crystallogr D. Biol Crystallogr D52, 842 (1996)) and Chimera (E. F. Pettersen, et al., J. Comput. Chem. 25, 1605 (2004)). The models were constructed from the known X-ray structures of the IRF-4-PU.1-DNA complex (C. R. Escalante et al., Mol Cell 10, 1097 (2002)) and the ATF/c-jun-IRF-3-DNA complex (M. Panne et al., EMBO J23, 4384 (2004)). Models of BATF (residues 28-87) and JunB (residues 266-326) were generated using the program Modeller and the structures of ATF and cJun as templates. Models of the DNA sites were made using the program 3DNA and modeled as B-type (X. J. L.a. W. K. Olson, Nucleic Acids Res 31, 5108 (2003)) using the sequences of Bcl11b (5′-TAGTGCAGAAATGAGTCAGAGA-3′) (SEQ ID NO: 68) and CTLA-4 (5′-GAGGTTTCGGGATGACTAATACT (SEQ ID NO: 69)). The IRF-4 and BATF/JunB molecules were placed on DNA by superposition of the individual binding sites from their respective Xray structures using the program LSQMAN. Models were energy minimized using Chimera.

### Example 1

### IRF4 Preferentially Binds to Sites with a Neighboring AP-1 Motif During T-Helper Cell Differentiation

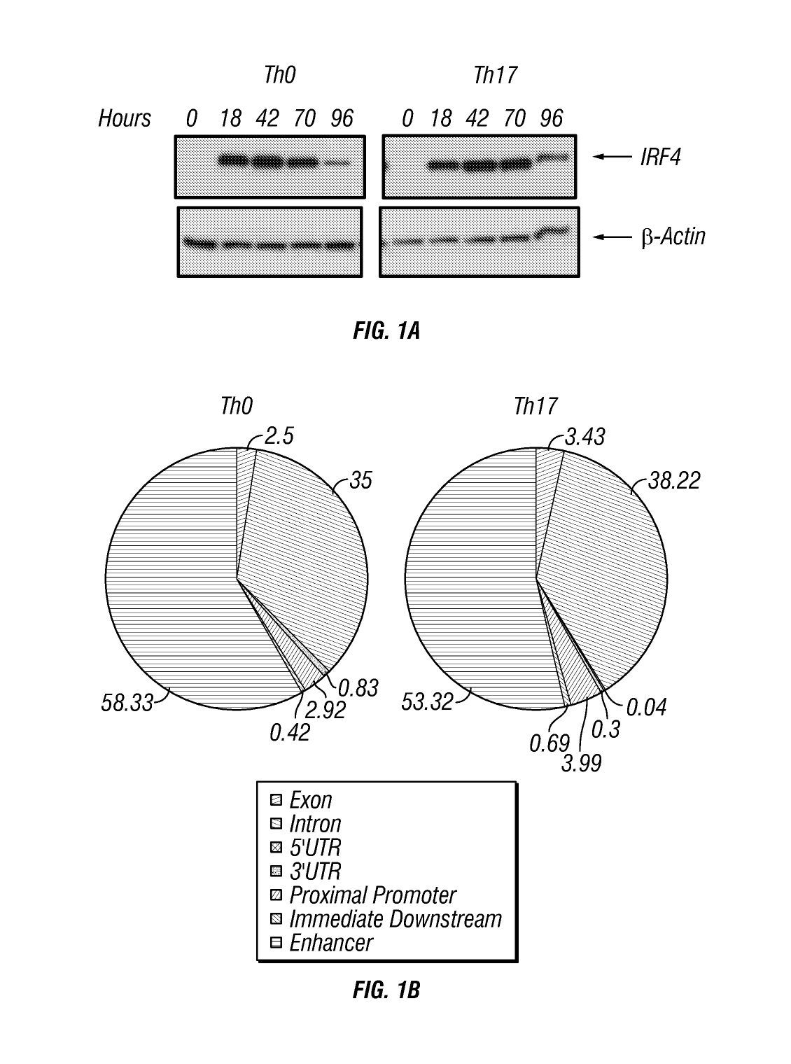

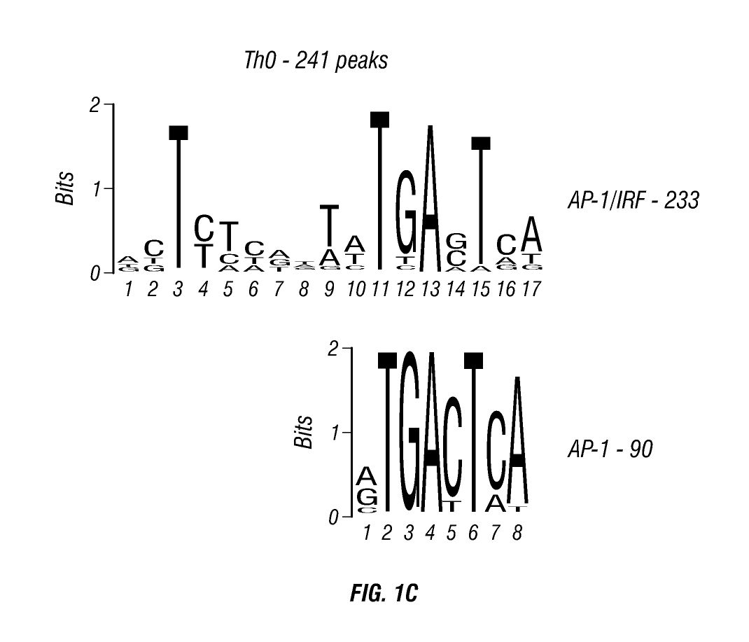

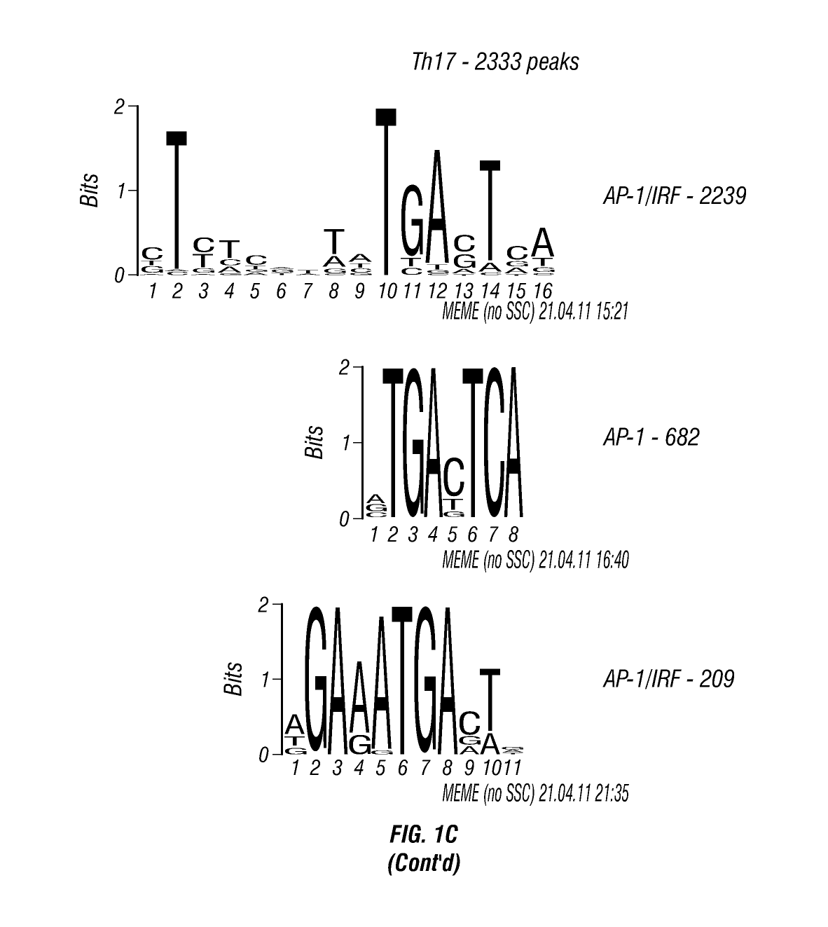

IRF4 is essential for the differentiation of Th17 cells (Brustle, Heink et al. 2007; Huber, Brustle et al. 2008; Mudter, Yu et al. 2011). To identify the genes targeted by IRF4 that are important for Th17 differentiation, a chromatin immunoprecipitation and sequencing (ChIP-seq) analysis of the IRF4 cistrome in Th17 cells was undertaken. IRF4 expression is induced by 18 hrs during the activation and differentiation of both Th0 and Th17 cells (FIG. 1a). Naïve CD4+ T-cells cultured under Th0 and Th17 polarizing conditions for 42 hrs were used to perform a ChIPseq analysis with anti-IRF4 antibodies. This identified 241 unique IRF4 targeted sequences in Th0 cells and almost 10-fold more (2333) unique IRF4 target sequences in Th17 cells. In both Th0 and Th17 cells, the majority of the binding sites in the IRF4 cistrome resided within intergenic (58.3% and 53.3%) or intronic (35% and 38.2%) regions (FIG. 1b). Surprisingly, analysis of the target regions using MEME revealed a composite AP-1/IRF motif as the most frequently occurring motif in both Th0 (˜97%) and Th17 (˜96%) cells (FIG. 1c). The AP-1 motif alone also occurred with a significant frequency in both Th0 and Th17 cells. In contrast to the first AP-1/IRF composite motif that has a 4 bp spacing between the IRF half site (TTTC) and AP-1 site (TGAG/CTCA), a distinct type of composite AP-1/IRF4 motif with no spacing between the IRF half site and AP-1 site was observed. Interestingly, a similar analysis at 18 hrs post induction did not reveal this second type of AP-1/IRF motif either in the Th17 or Th0 cells whereas the AP-1/IRF motif with a 4 bp gap was present at 18 hrs in the IRF4 target sequences in Th17 cells only, but with a much lower frequency (˜29%) (data not shown).

### Example 2

### IRF-4 Cistrome in Th17 Cells Reveals Enrichment of AP-1-IRF Composite Motifs

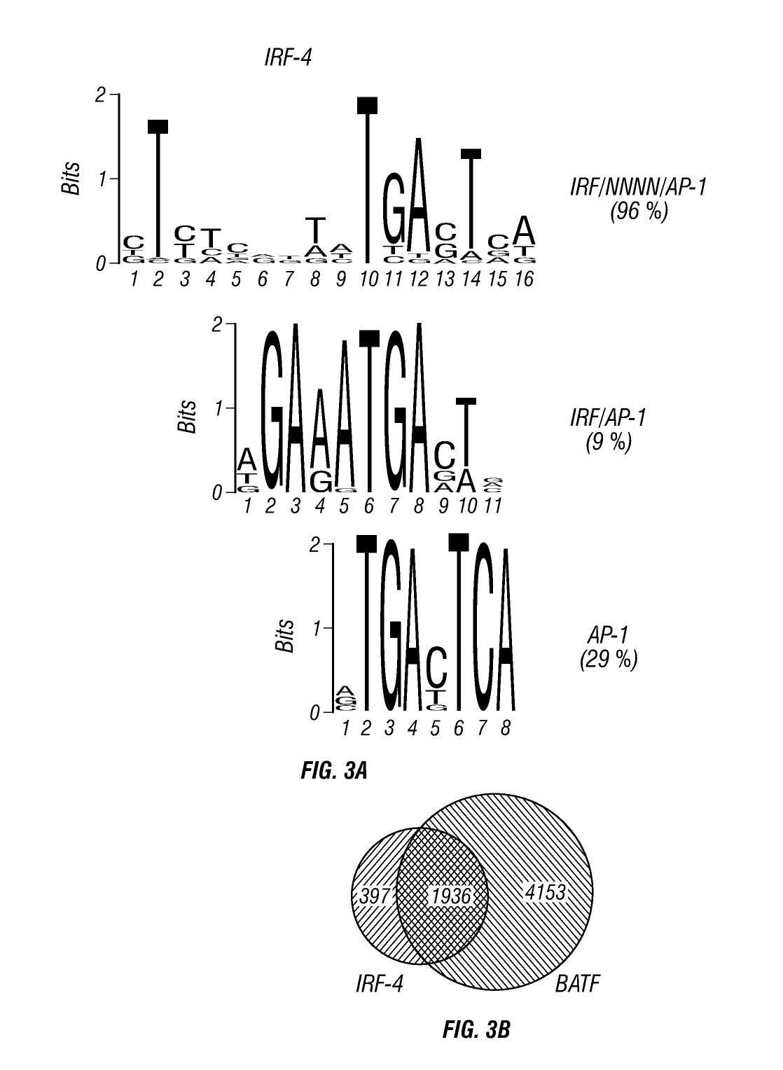

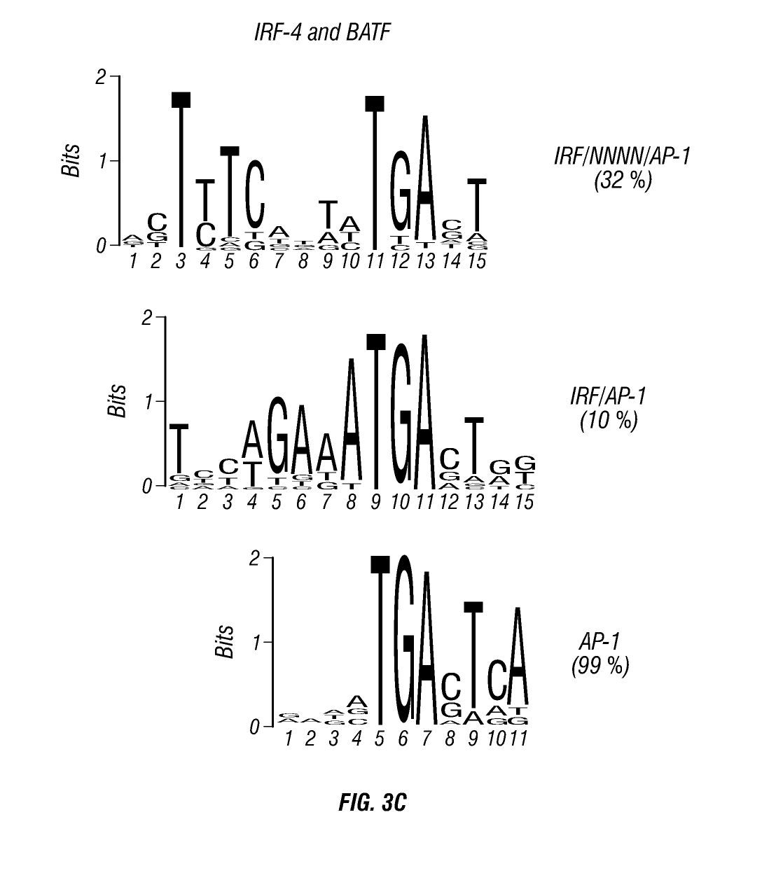

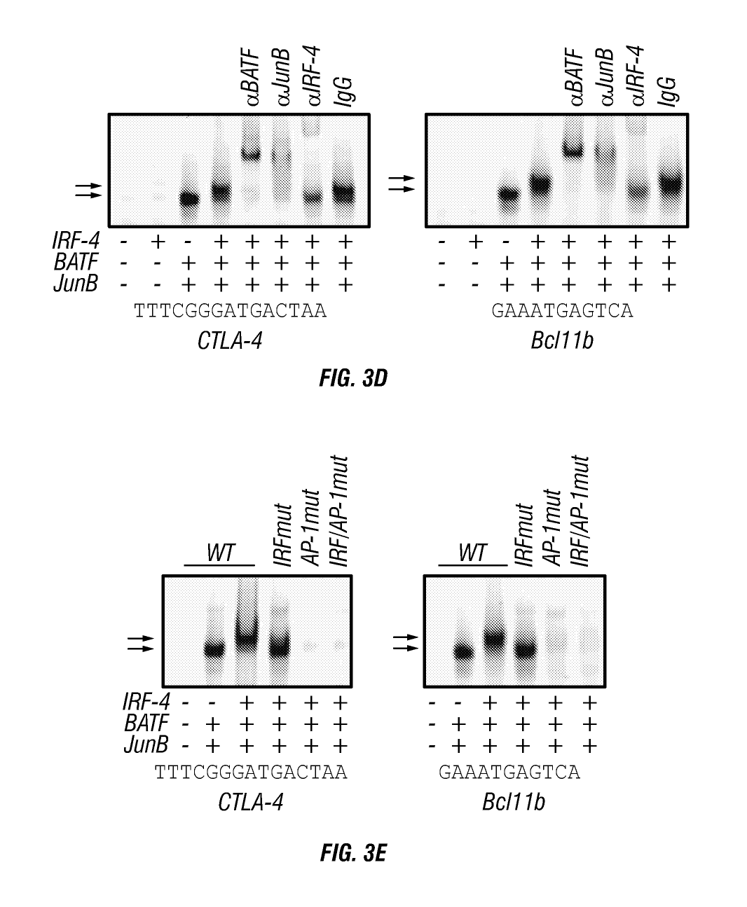

It was reasoned that ChIPseq analysis could be used to further identify the nature of interaction partners for IRF-4 in T cells as the DNA binding motif of such a transcription factor(s) should be juxtaposed with the IRF motif in a stereo-specific manner within a large set of targeted sequences. The utility of this method was tested by performing ChIPseq with IRF-4 in the J558L B cell line, which expresses PU.1 protein. As anticipated, the EICE motif occurred with highest frequency in the targeted regions (FIG. 2a). To analyze DNA sequences targeted by IRF-4 in Th17 cells, naïve CD4+ T-cells that were activated with anti-CD3 and anti-CD28 were utilized in the presence of polarizing cytokines. IRF-4 expression was induced under these conditions and reached maximal levels between 42 and 70 h (FIG. 2b). ChIPseq analysis with IRF-4 antibodies identified 2333 unique sequences. Intriguingly, de novo motif analysis (MEME) of DNA sequences underlying the peak maxima revealed AP-1 motifs, many of which were juxtaposed with IRF motifs with either a 4 bp (IRF/NNNN/AP-1) or 0 bp (IRF/AP-1) spacing (FIG. 3a). These results suggested that IRF-4 is recruited to such composite motifs by interactions with AP-1 family members.

### Example 3

### BATF Co-Targets IRF4-Bound Sequences in Th17 Cells

Since BATF is an AP-1 family transcription factor that is required for Th17 differentiation (B. U. Schraml et al., Nature 460, 405 (2009)), we hypothesized that it may represent a novel interaction partner for IRF-4. If so, then ChIPseq should not only reveal binding to IRF-4 targeted regions but also enrich for the composite AP-1-IRF motifs. A time-course analysis of BATF expression post activation and differentiation of Th0 and Th17 cells revealed that unlike IRF4, BATF is only induced in Th17 cells with a peak in expression at 42 hrs (data not shown). A ChIPseq analysis with anti-BATF antibodies in Th17 cells at 42 hrs post induction was therefore performed. The BATF cistrome appears to be larger than the IRF4 cistrome in Th17 cells at 42 hrs. Such analysis with BATF antibodies identified 6089 sequences, the majority of which (>90%) contained an AP-1 motif (FIG. 2c). Importantly, the predicted AP-1-IRF composite motifs (4 and 0 bp spacing) were present in 18% and 16% of the targeted sequences, respectively. Analysis of coincident peaks in the IRF-4 and BATF ChIPseq datasets using a distance cutoff of 100 bp from the maxima of each peak resulted in the identification of 1936 co-targeted regions (FIG. 3b). Strikingly, ˜83% of the IRF-4 target sequences were co-bound by BATF suggesting a BATF-mediated recruitment of IRF-4 to presumptive regulatory elements.

MEME analysis of the co-targeted sequences clearly revealed the two distinct AP-1-IRF composite motifs (FIG. 3c). Analysis of individual sequences showed that 158 could be stringently described as TTTC(N4)TGA(G/C)T(C/A)A (SEQ ID NO: 3) or GAAATGA(G/C)T(C/A)A (SEQ ID NO: 4) (Table 4 below). If one relaxes the stringency of the IRF core motif based on the MEME analysis then a substantially larger number of composite motifs (˜42%) are apparent. Thus a large majority of IRF-4 targeted sequences in Th17 cells are co-bound by BATF (1936/2333) and nearly half of these contain composite AP-1-IRF motifs representing alternate configurations distinguished by the spacing and orientation of the IRF motif.

TABLE 4
AICE motifs (SEQ ID NOS 70-161, respectively,
in order of appearance).

| Genomic location | 4 bp spacing |
|---|---|
| chr15 80413204 | TTTCGGTATGAGTCA |
| chr8 107645197 | TTTCAGTATGAGTCA |
| chr9 32572834 | TTTCAGTATGAGTCA |
| chr15 66693605 | TTTCGGAATGAGTCA |
| chr16 36684843 | TTTCGAGATGACTCA |
| chr7 107379308 | TTTCAAAATGAGTCA |
| chr1 146349552 | TTTCATTTTGACTCA |
| chr10 100005815 | TTTCTTTATGAGTCA |
| chr14 119379174 | TTTCAGTCTGACTCA |
| chr8 109318957 | TTTCAGTCTGACTCA |
| chr5 53963878 | TTTCTAAATGACTCA |
| chr9 110175027 | TTTCTAAATGACTCA |
| chr15 11940228 | TTTCGCTTTGACTCA |
| chr1 90030706 | TTTCATTCTGAGTCA |
| chr10 87868648 | TTTCATTCTGAGTCA |
| chr15 58922472 | TTTCCAAATGACTCA |
| chr10 39199363 | TTTCAGTTTGAGTCA |
| chr16 51650399 | TTTCCGTATGAGTCA |
| chrX 7682327 | TTTCATATTGACTCA |
| chr4 8682738 | TTTCTGAATGACTCA |
| chr1 64238319 | TTTCTTTTTGACTCA |
| chr11 74786977 | TTTCTTTCTGACTCA |
| chr3 30851325 | TTTCTTTCTGACTCA |
| chr11 86437977 | TTTCACTCTGACTCA |
| chr11 4705150 | TTTCTTTTTGAGTCA |
| chr5 103868356 | TTTCCTTTTGAGTCA |
| chr1 53902303 | TTTCAGATTGAGTCA |
| chr1 132907245 | TTTCGTTGTGAGTCA |
| chr11 76686242 | TTTCTGTCTGAGTCA |
| chr6 146858428 | TTTCATTGTGACTCA |
| chr17 32607933 | TTTCAAGTTGACTCA |
| chr15 31472560 | TTTCTCTCTGACTCA |
| chr4 95353678 | TTTCACATTGACTCA |
| chr9 57822257 | TTTCTCTTTGACTCA |
| chr2 25278022 | TTTCCCTTTGACTCA |
| chr5 141470541 | TTTCAAAGTGACTCA |
| chr15 77616379 | TTTCTATGTGACTCA |
| chr2 10272950 | TTTCTTGTTGAGTCA |
| chr7 35127321 | TTTCGGGGTGACTCA |
| chr11 87563234 | TTTCTGGTTGAGTCA |
| chr7 29229516 | TTTCATCCTGACTCA |
| chr15 55874229 | TTTCCAAATGACTCA |
| chr7 82941513 | TTTCAGACTGACTCA |
| chr1 36382964 | TTTCGCAGTGAGTCA |
| chr10 42434664 | TCCCGAAATGACTCA |
| chr2 26358450 | TTCCGAAATGAGTCA |
| chr16 17024322 | TCTGGAAATGACTCA |
| chr14 26681419 | TCACGAAATGACTCA |
| chr13 20228793 | TCCTGAAATGAGTCA |
| chr4 59695563 | TCCTGAAATGAGTCA |
| chr1 55487650 | TCCAGAAATGACTCA |
| chr13 9766757 | TATCGAAATGACTCA |
| chr5 140993919 | TCCAGAAATGAGTCA |
| chr7 107598977 | TCATGAAATGAGTCA |
| chr10 95926519 | TTATGAAATGACTCA |
| chr13 97257373 | TCAGGAAATGAGTCA |
| chr4 87766229 | CTCAGAAATGACTCA |
| chr10 90491945 | CAATGAAATGAGTCA |
| chr16 32915956 | TACTGAAATGACTCA |
| chr8 13624093 | TCGGGAAATGACTCA |
| chr9 119175063 | TTGTGAAATGAGTCA |
| chr11 99011990 | CCCCGAAATGAGTCA |
| chr16 38403942 | TATGGAAATGAGTCA |
| chr11 53160962 | TGCAGAAATGACTCA |
| chr12 109208372 | TGCAGAAATGAGTCA |
| chr2 50106509 | TGCAGAAATGAGTCA |
| chr18 80425305 | GTTTGAAATGAGTCA |
| chr9 63601417 | TGGTGAAATGACTCA |
| chr1 64347897 | GCTAGAAATGAGTCA |
| chr6 28202956 | TGGTGAAATGAGTCA |
| chr2 134915568 | CTCAGAAATGACTCA |
| chr19 29224317 | CCCAGAAATGAGTCA |
| chr3 130872280 | ATTGGAAATGACTCA |
| chr9 123903315 | AGCAGAAATGACTCA |
| chr18 39170272 | TACAGAAATGACTCA |
| chr8 109318957 | TAACGAAATGAGTCA |
| chr16 20465006 | GGAAGAAATGACTCA |
| chr13 113233536 | ACTGGAAATGAGTCA |
| chr5 63207183 | TTCAGAAATGACTCA |
| chr5 118854517 | CGTAGAAATGACTCA |
| chr11 79607456 | TGAGGAAATGACTCA |
| chr15 37537482 | CTGAGAAATGAGTCA |
| chr8 116876118 | GGAGGAAATGACTCA |
| chr15 58361942 | AGCAGAAATGACTCA |
| chr6 91017491 | CAGAGAAATGACTCA |
| chr6 146470139 | AGCAGAAATGACTCA |
| chr4 154882913 | TAGAGAAATGAGTCA |
| chr11 53470542 | GAAAGAAATGACTCA |
| chr11 116295145 | AGAGGAAATGAGTCA |
| chr15 94405450 | AGCAGAAATGACTCA |
| chr16 32010105 | AGCAGAAATGACTCA |
| chr16 92825856 | GAAGGAAATGACTCA |

### Example 4

### Cooperative Binding of IRF-4 and BATF/JunB on AICE Motifs

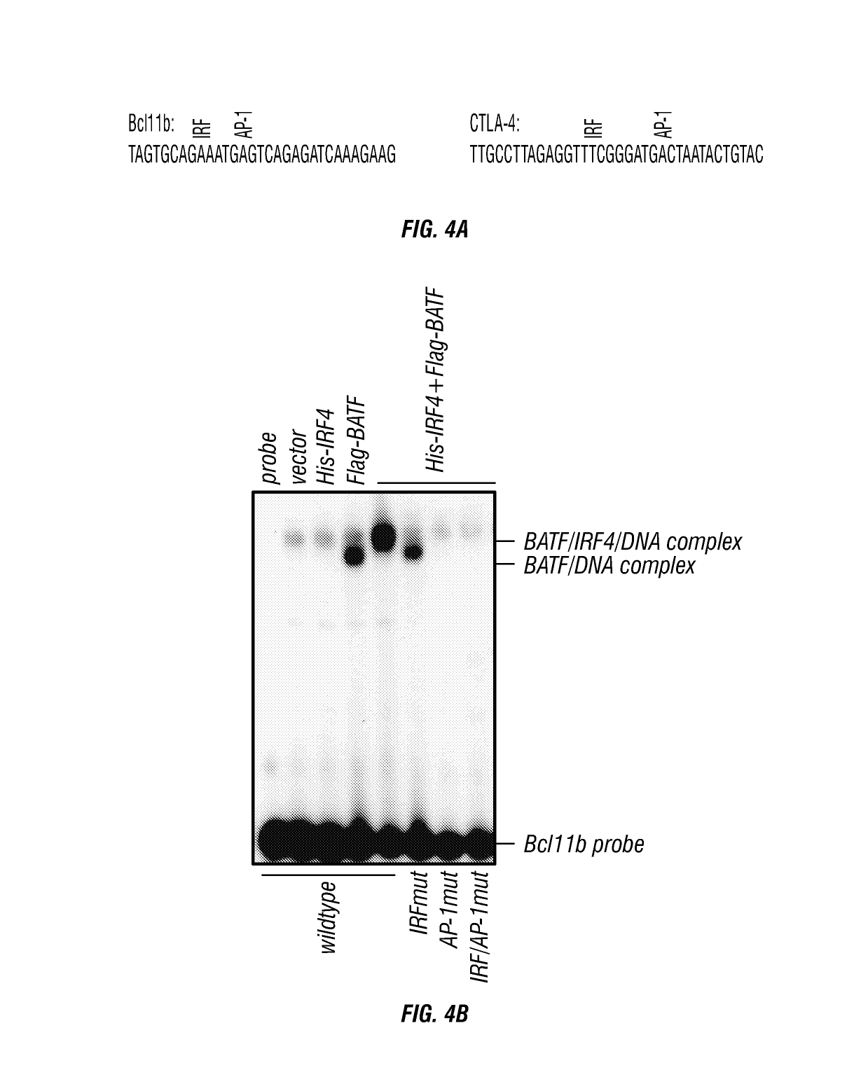

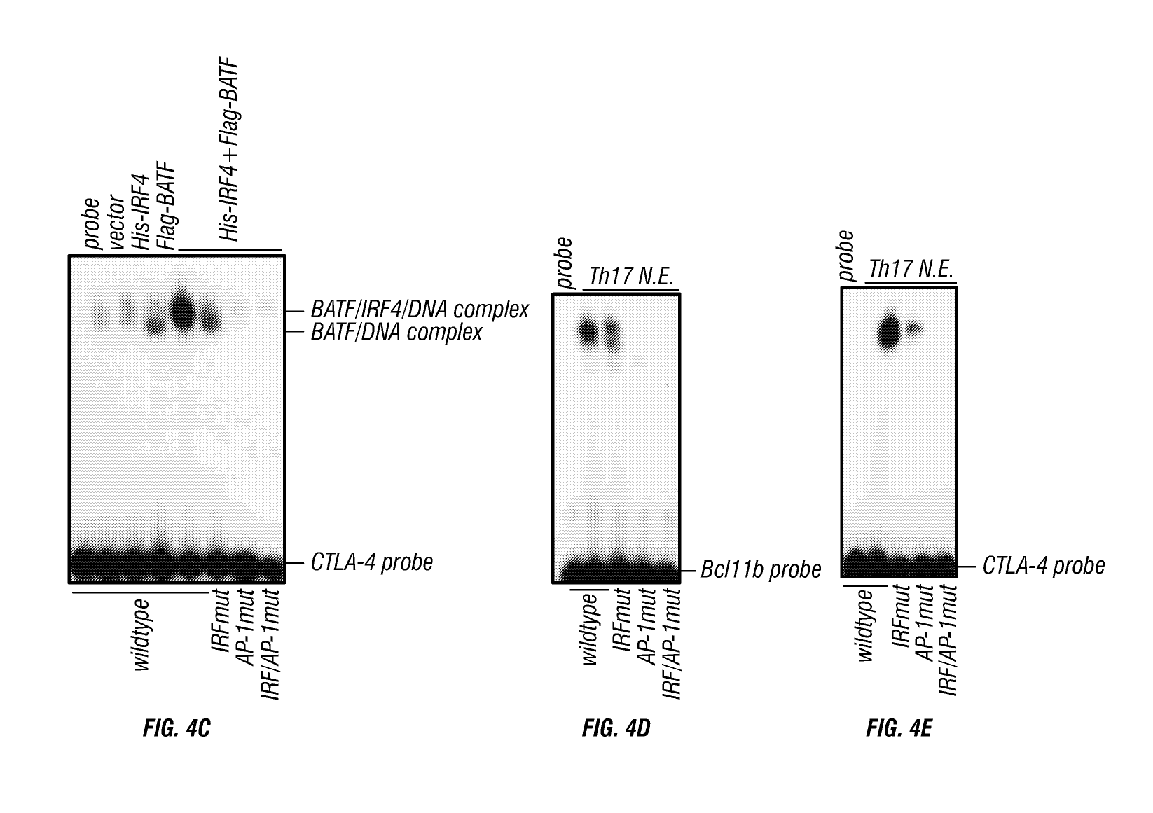

To test whether IRF4 and BATF can cooperatively bind in-vitro to some of the identified co-targeted sequences in Th17 cells, electromobilty gel shift assays (EMSAs) were performed. Nuclear extracts from 293T cells over-expressing IRF4 or BATF were incubated with probes containing IRF and AP-1 motifs with different spacing requirements from intronic regions within the CTLA-4 and Bcl11b genes (FIG. 4a-c). In line with previous reports that IRF4 binds DNA weakly by itself (Eisenbeis, Singh et al. 1995; Nagulapalli and Atchison 1998; Brass, Zhu et al. 1999) no specific protein-DNA complexes were detectable with extracts over-expressing IRF4 (FIG. 4b,c). However, a specific protein-DNA complex was observable with extracts over-expressing BATF (FIG. 4b, c). Incubation of IRF4- and BATF-expressing extracts with the DNA probes resulted in a slower migrating protein-DNA complex (FIG. 4b, c) and the increased intensity of these novel complexes suggested cooperative binding of IRF4 and BATF to the two types of composite AP-1/IRF motifs. Mutation of the IRF, the AP-1 motif or the combination of both disrupted cooperative binding verifying that the IRF4/BATF/DNA ternary complex depends on the predicted IRF and AP-1 motifs. More precisely, mutation of the IRF motif still allows BATF-DNA complex formation (FIG. 4a, b), whereas mutation of the AP-1 motif alone or in combination with the mutated IRF motif results in loss of ternary complex formation (FIG. 4a, b)).

To test whether the IRF4/BATF/DNA complex formation can be observed in Th17 cells nuclear extracts isolated from such cells were incubated with the Bcl11b and CTLA-4 probes. Gel shift assays revealed protein-DNA complexes probes (FIG. 4d, e), which were disrupted with probes mutated for the IRF motif, AP-1 motif or both (FIG. 4d, e), strongly suggesting that these complexes involved IRF and AP-1 subunits. Furthermore these complexes are not observed in nuclear extracts prepared from IRF-4−/− T helper cells and are supershifted with an anti-BATF antibody (data not shown) thereby confirming that they contain IRF-4 and BATF.

To test if IRF-4 and BATF can bind to the composite motifs identified by ChIPseq analysis, electromobility shift assays (EMSAs) were also performed using nuclear extracts from Th17 cells and DNA probes, representing the alternate configurations, derived from intronic regions within the CTLA-4 and Bcl11b genes (Fig. S2d,e). Both DNA probes gave rise to protein-DNA complexes that co-migrated and antibody supershifting verified IRF-4 and BATF as constituents. Given that BATF has been shown to form a heterodimer with JunB in Th17 cells (B. U. Schraml et al., Nature 460, 405 (2009)) the presence of JunB in these complexes was tested for. JunB antibodies supershifted the complexes suggesting that an AP-1 heterodimer comprised of JunB and BATF co-binds with IRF-4. To demonstrate DNA dependent assembly, each protein was individually expressed in 293T cells and then used for gel shift assays. Given the weak DNA binding affinity of IRF-4, no specific protein-DNA complexes were detected with it (FIG. 3d). On the other hand, a complex was observed in reactions containing BATF and JunB, reflecting binding of a heterodimer to the AP-1 motif. Addition of IRF-4 to the BATF/JunB binding reactions resulted in its recruitment to the DNA probes generating slower migrating protein-DNA complexes. Antibody supershift experiments verified the protein constituents of the complexes. These data demonstrate that IRF-4 is recruited to AP-1-IRF composite motifs by a DNA bound BATF/JunB heterodimer, a molecular mechanism reminiscent of IRF-4 recruitment to EICE motifs by DNA bound PU.1.

To verify that a BATF/JunB heterodimer and IRF-4 assemble on the two types of AP-1-IRF motifs by interacting with the AP-1 and IRF sites respectively, DNA probes, in which the individual or both sites had been mutated, were utilized (FIG. 3e). Mutation of the IRF site resulted in a faster migrating complex that co-migrated with one generated by the BATF/JunB heterodimer. On the other hand mutation of the AP-1 site resulted in loss of both protein-DNA complexes. A similar result was observed with mutation of both the AP-1 and IRF sites. Thus binding of a BATF/JunB heterodimer to the AP-1 site functions to recruit IRF-4 to DNA via the latter's recognition of the IRF site in the composite sequences. Surprisingly, a similar mechanism of binding is observed on two structurally distinct AP-1-IRF composite motifs, which differ in the spacing and orientation of the IRF site. By analogy with the Ets-IRF composite elements (EICE) characterized in earlier work, the AP-1-IRF composite elements are designated as

AICE motifs.

The consequences of mutations in AICE motifs were also analyzed on the assembly of protein-DNA complexes in nuclear extracts from Th17 cells. Mutation of the AP-1 motif resulted in loss of the protein-DNA complexes (FIG. 2f). In contrast mutation of the IRF site disrupted the major protein-DNA complex observed in Th17 cells, but resulted in two protein-DNA complexes, one of which migrated more slowly and the other with faster mobility than the parent complex. As expected the faster migrating complex was supershifted with antibodies recognizing BATF and JunB. The slower complex was supershifted with pan anti-Fos and anti-JunB reagents. Neither complex was affected by the addition of anti-IRF-4. These results indicate that either BATF/JunB or Fos/JunB heterodimers can bind to the AP-1 site within AICE motifs in Th17 cells. Furthermore, they suggested that IRF-4 is preferentially recruited to AICE motifs by a DNA bound BATF/JunB heterodimer in contrast with an alternate AP-1 complex such as

a Fos/JunB heterodimer.

### Example 5

### Assembly of IRF-4/BATF/JunB Complexes on Key Th17 Genes

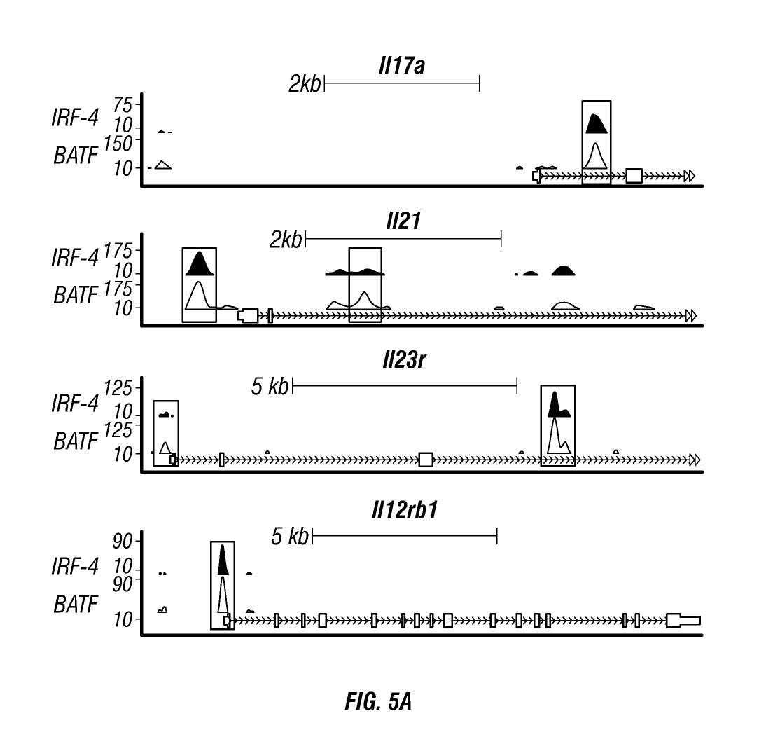

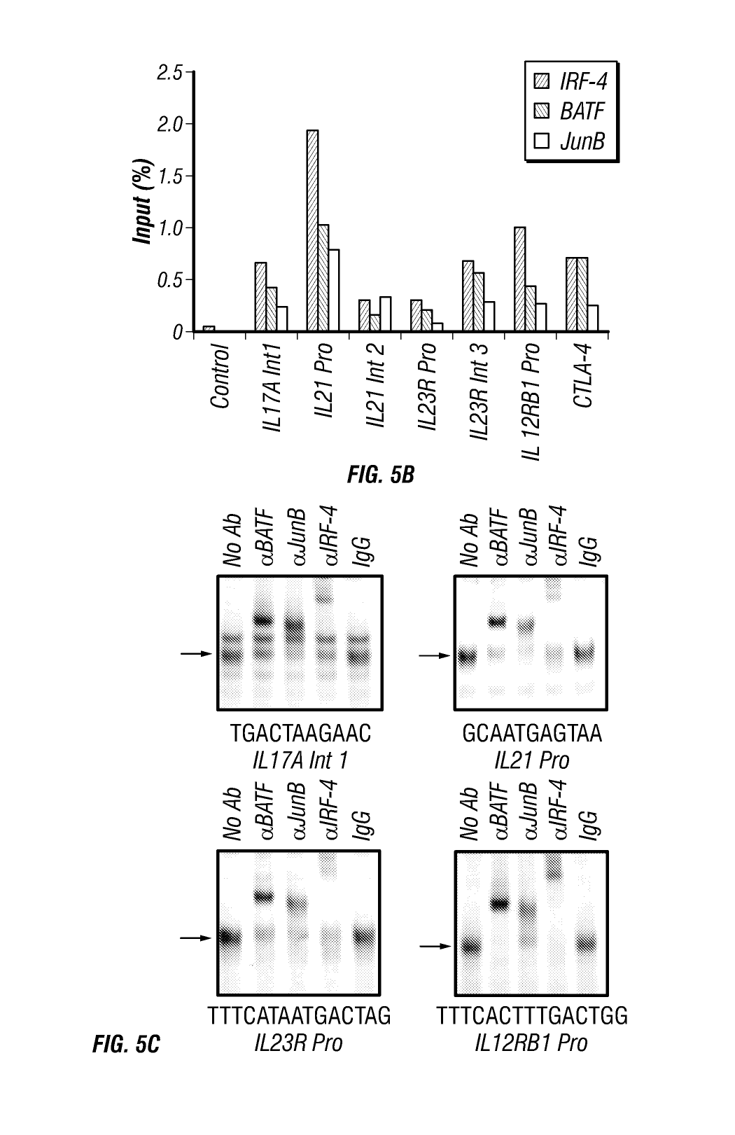

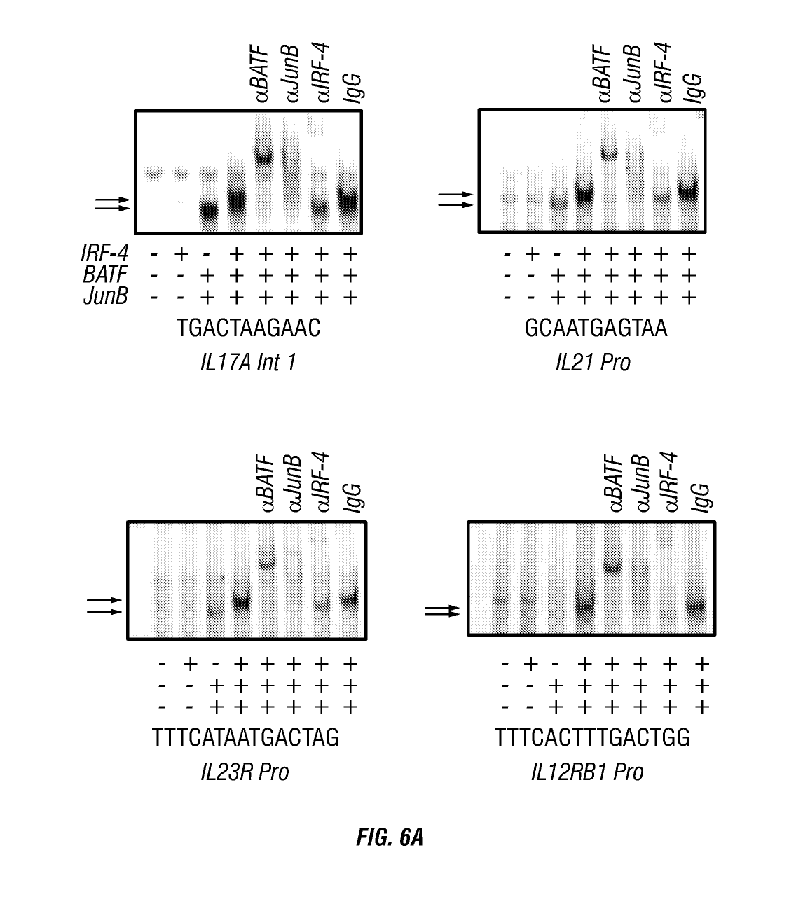

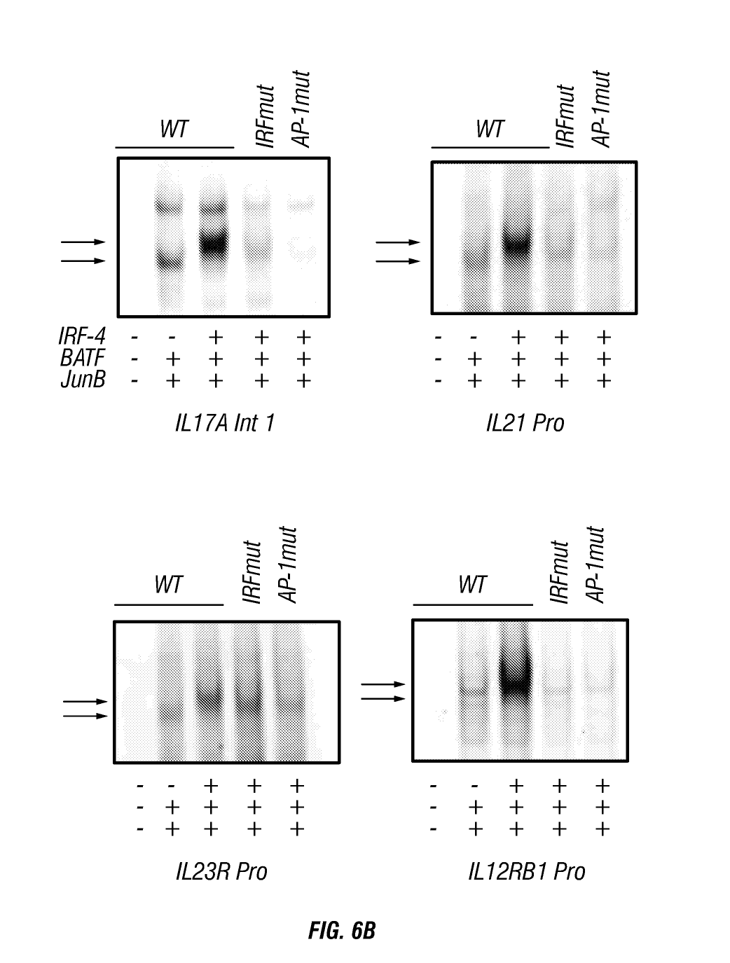

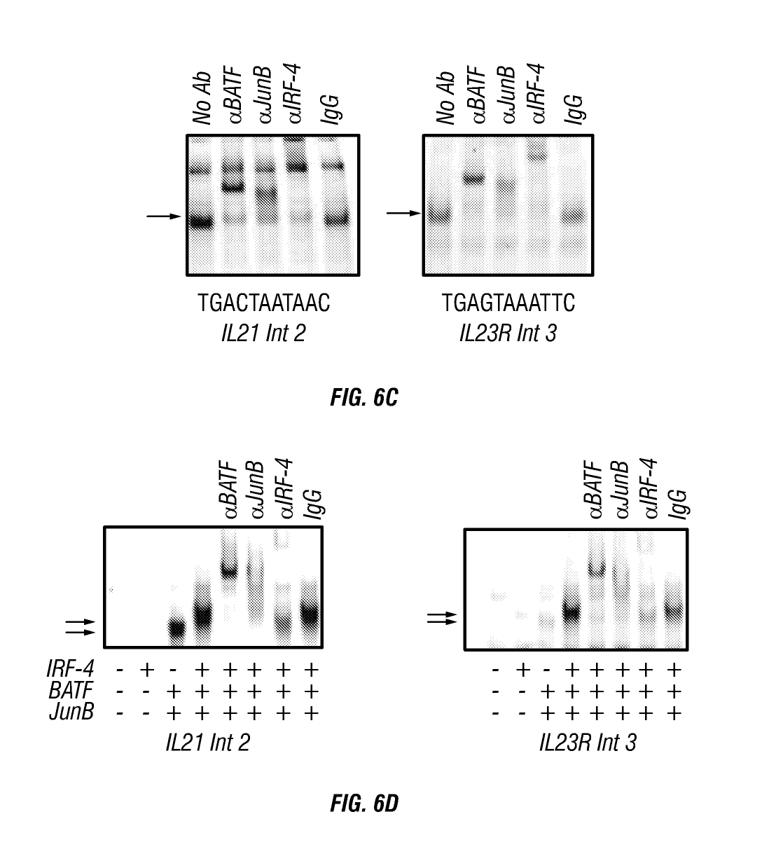

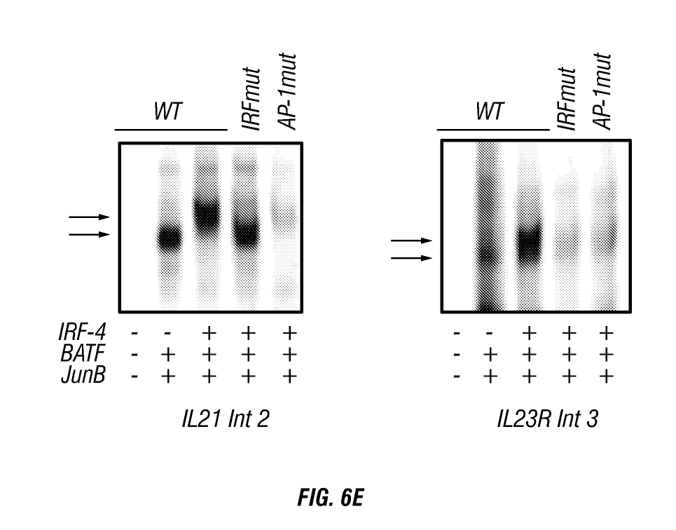

To determine if IRF-4/BATF/JunB complexes bind to presumptive regulatory sequences in genes required for the generation and/or functioning of Th17 cells, the Il17, Il21, Il22, Il23r and Il12rb1 loci were initially focused on. Each of these loci was found to contain one or more coincident binding peaks for IRF-4 and BATF that were either positioned in promoters and/or within intronic regions (FIG. 5a). The peak in the 1122 gene was located approximately 30 kb upstream of the promoter (data not shown). ChIP assays verified not only the binding of IRF-4 and BATF to these regions but also demonstrated the co-binding of JunB (FIG. 5b). Analysis of the DNA sequences underlying these peaks revealed that three had discernable AICE motifs whereas the remaining three did not (FIG. 5c, FIG. 6c). Remarkably, all six DNA probes gave rise to a predominant co-migrating complex with Th17 nuclear extracts that was supershifted with anti-BATF, anti-JunB and anti-IRF-4 (FIG. 5c, FIG. 6c). With each probe the BATF/JunB heterodimer was seen to bind in the absence of IRF-4 although to a varied extent depending on the nature of the AP-1 site (FIG. 6a, 6d). Importantly, the BATF/JunB complex recruited IRF-4 on to each target sequence. In a reciprocal manner IRF-4 was seen to enhance the binding of BATF/JunB, particularly on sequences that had variant, lower affinity AP-1 sites, consistent with a cooperative mechanism of assembly that is DNA directed. In case of the discernable AICE motifs mutation of the AP-1 and IRF sites had predictable consequences on the formation of the BATF/JunB and BATF/JunB/IRF-4 complexes (FIG. 6b). Surprisingly, for the remaining three sequences mutation of nucleotides on the 3′-end of the AP-1 site led to loss of IRF-4 recruitment (FIG. 6b, e). These results demonstrate that IRF-4/BATF/JunB complexes target a key set of Th17 genes and cooperatively assemble on structurally diverse AICE motifs.

### Example 6

### Delineation of IRF-4/BATF Gene Regulatory Modules in Th17 Cells

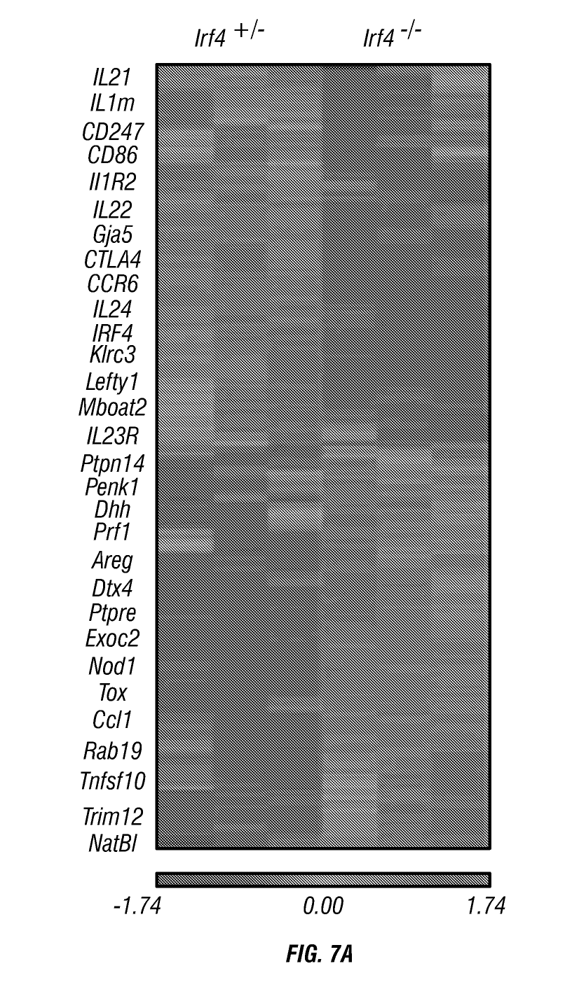

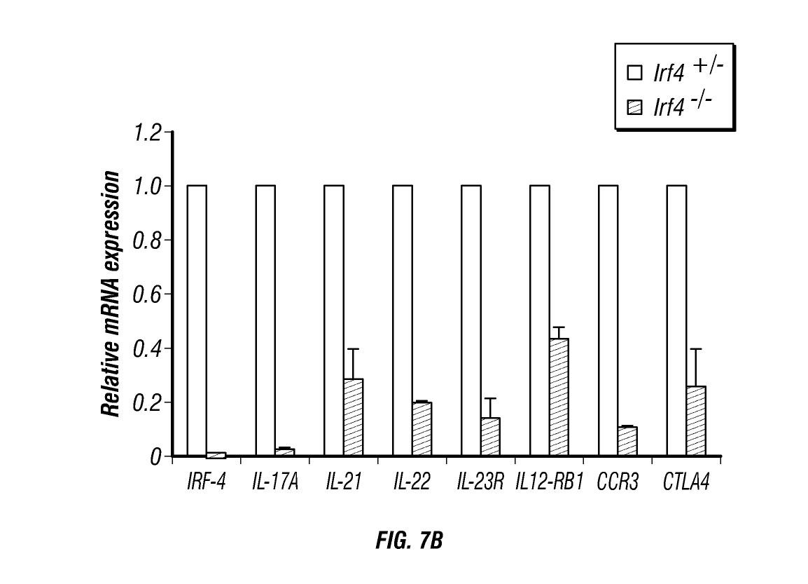

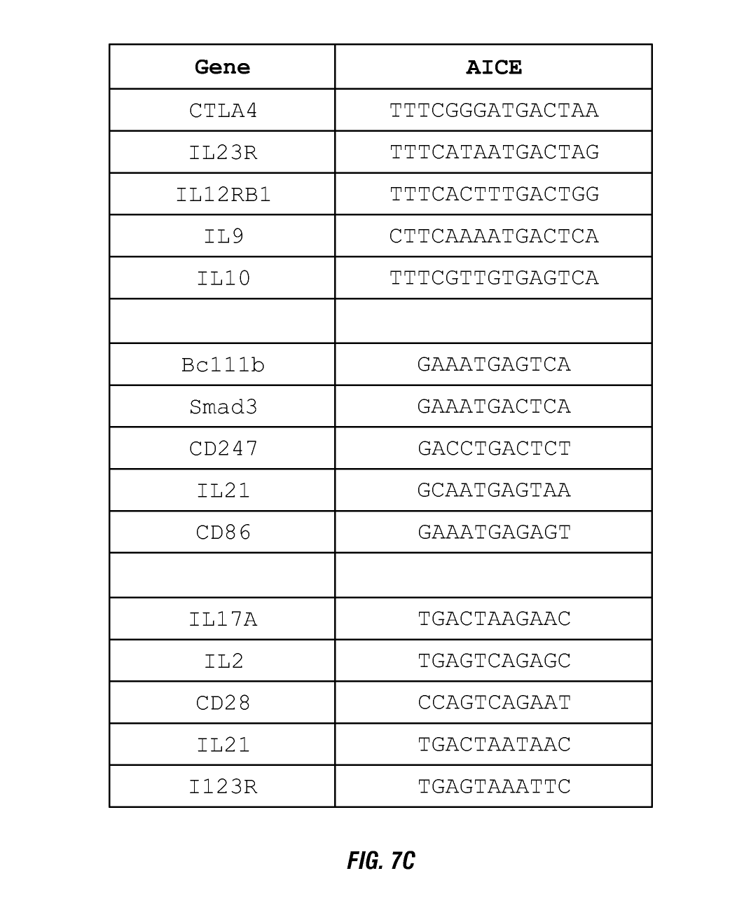

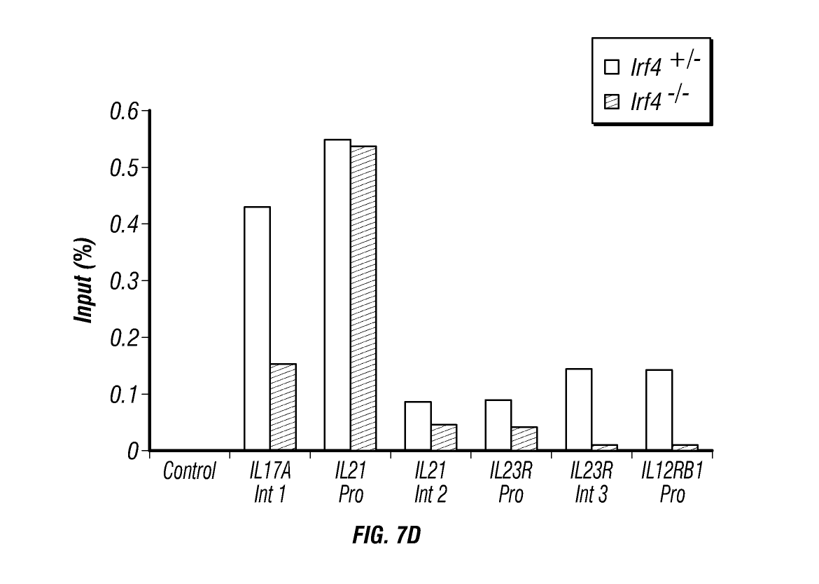

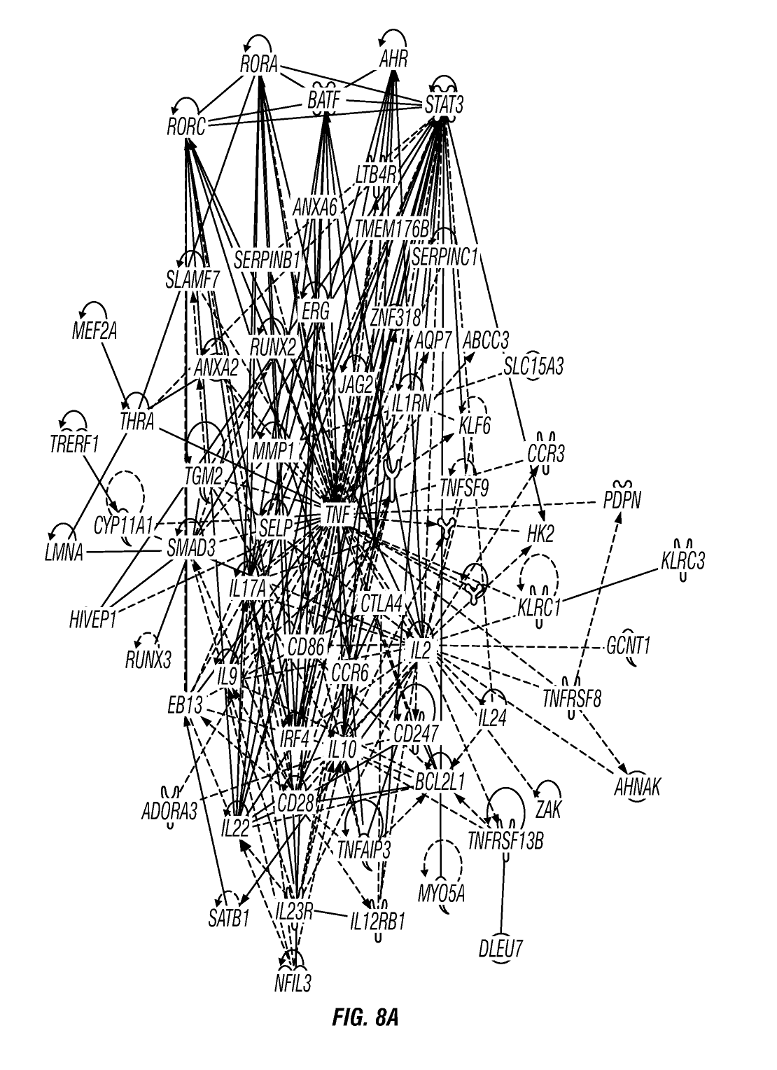

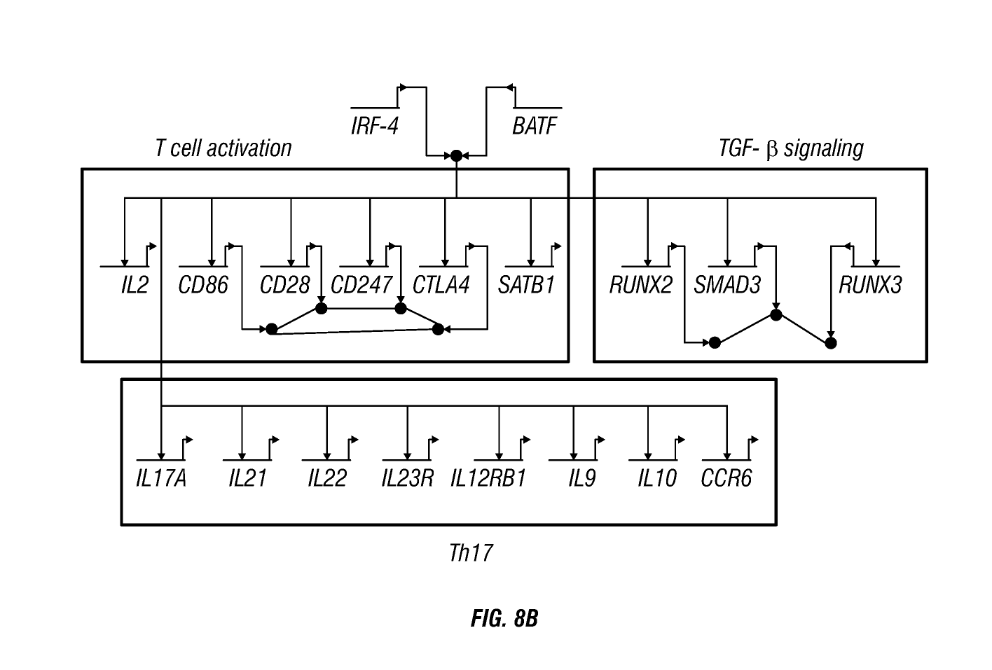

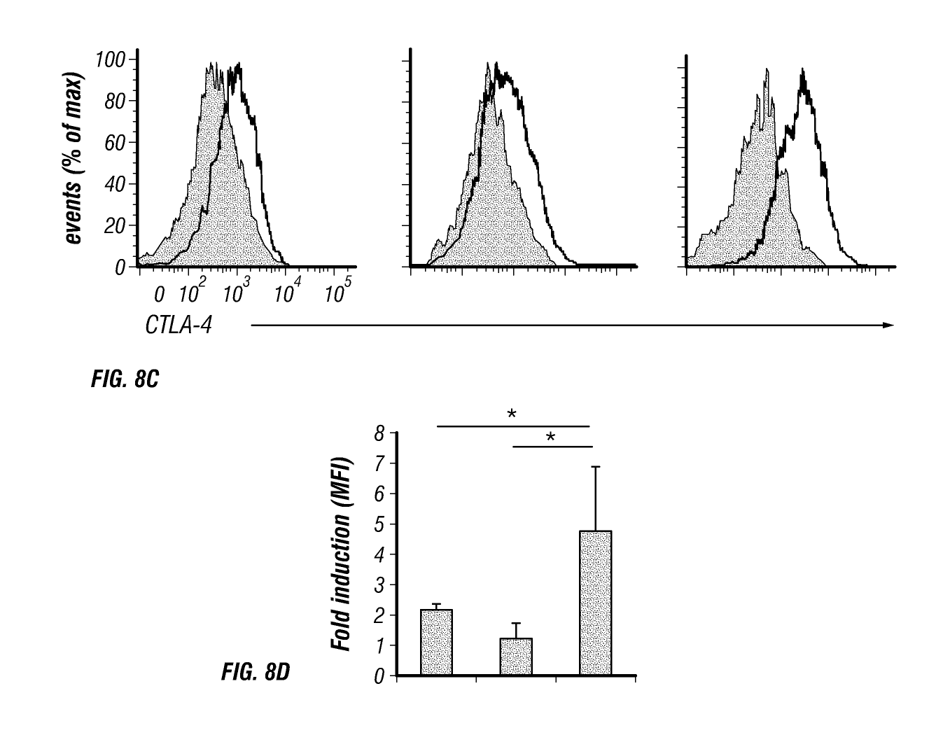

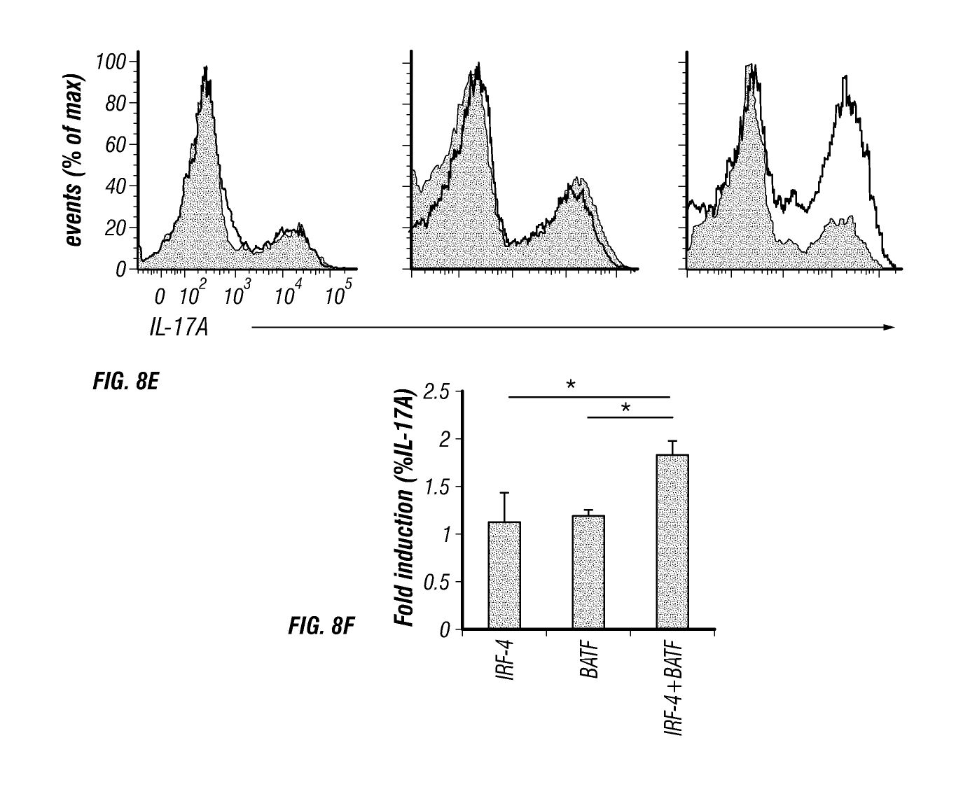

To identify genes regulated by IRF-4 and targeted through its recruitment by BATF complexes, genome-wide expression analysis of Irf4^{-/-} T cells polarized under Th17 conditions was performed (FIG. 7a). As described previously, there was a profound defect in the expression of Il17a, Il21, Il22 and Il23r genes in Irf4^{-/-} cells (FIG. 7b). In addition, these cells were also defective in expression of 1l12rb1, encoding a subunit of IL-23R as well as Ccr6 and also genes involved in T-cell activation including Cd86, Cd247, Cd28, Ctla4 and Il2 (FIG. 7a). Of the 362 genes that were positively regulated by IRF-4 during Th17 differentiation, 154 (˜42.5%) contained coincident IRF-4 and BATF peaks. Ingenuity Pathway Analysis (IPA) database revealed that 65 of these 154 genes formed a highly interconnected network (FIG. 8a, blue edges and nodes). Notably, this network did not include genes encoding the other transcription factors required for Th17 differentiation, namely Rorc, Rora, Ahr and Stat3 (A. Peters, Y. Lee, V. K. Kuchroo, Current Opinion in Immunology 23, 702 (2011); A. Kimura, T. Naka, K. Nohara, Y. Fujii-Kuriyama, T. Kishimoto, PNAS 105, 9721 (2008); C. Dong, Experimental & Molecular Medicine 43, 1 (2011)). However, if these regulatory factors were introduced into the network (FIG. 8a, orange edges and nodes) they were seen to make a large number of connections. Therefore, IRF-4/BATF complexes appear to target and regulate a key set of genes in Th17 cells independently of but in concert with Rorα, Rorγt, AHR and/or STAT-3. Upon closer inspection of this network of genes, three types of regulatory modules could be discerned (FIG. 8b). The first termed the ‘T-cell activation module’ comprised of genes that play a key role during T-cell activation and includes Il2, Cd86, Cd28, Cd247, Ctla4 and Satb1. A second, termed the ‘Th17 module’, consisted of genes that are preferentially expressed by Th17 cells, including Il17a, Il21, Il22, Il23r and Il12rb1. Finally, Smad3, Runx2 and Runx3 were designated the ‘TGF-” module’, given their functionality in this signaling pathway that is required for Th17 differentiation (J. J. O'Shea et al., Microbes and Infection/Institut Pasteur 11, 599 (2009)). It is noted that most of these genes contain identifiable AICE motifs in the DNA regions that are co-bound by IRF-4 and BATF (FIG. 7c). Given the evidence for cooperative binding of IRF-4 and BATF/JunB complexes on AICE motifs (FIG. 5, 6), it was tested if loss of IRF-4 resulted in impaired binding of BATF to AICE motif containing target sequences in vivo. BATF binding in vivo was reduced to varying degrees but more severely at regions containing AICE motifs with low affinity AP-1 sites (FIG. 7d). It is noted that IRF-4 co-binding with BATF has also been observed in ChIPseq analyses of Th17 cells by the Littman laboratory and the targeting of IRF-4 to a large set of genomic sequences is lost in Batf^{-/-} T cells (personal communication). Thus, without being bound by theory, it is proposed that in the context of Th17 cell differentiation, BATF containing AP-1 complexes cooperatively assemble with IRF-4 on AICE motifs and activate the expression of key genes in a combinatorial manner with Rorγt, AHR and/or STAT-3.

### Example 7

### IRF4 and BATF Cooperatively Induce Expression of CTLA-4 and ICOS Genes

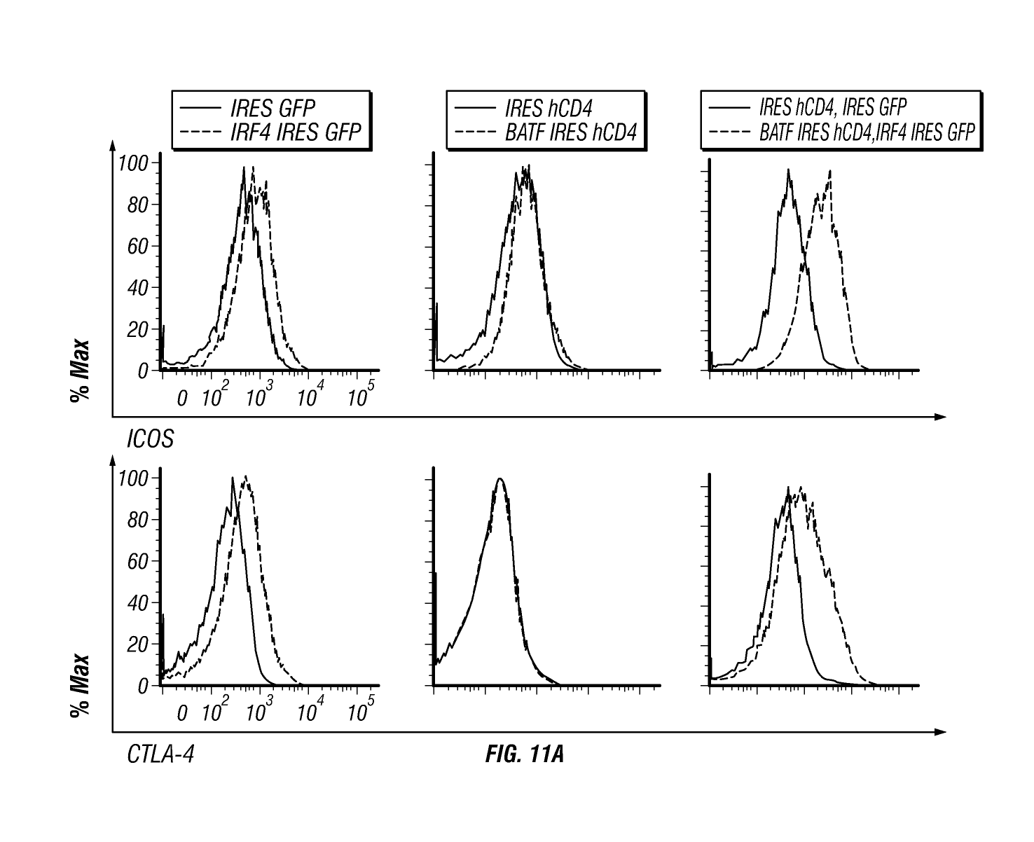

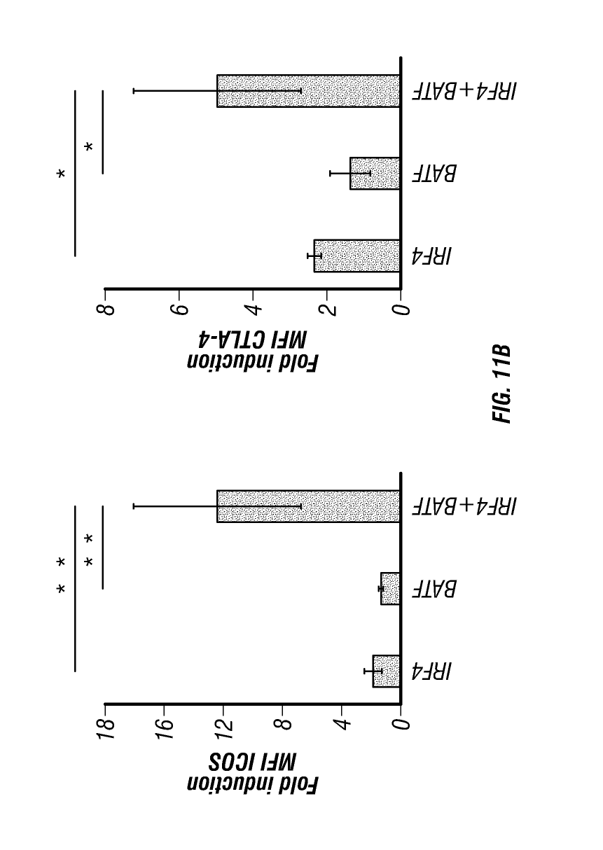

It has been demonstrated above that BATF co-targets about 83% of IRF4-bound sequences and that these two transcription factors can bind in-vitro to selected sequences containing composite AP-1/IRF-4 motifs. It was next tested whether IRF-4 and BATF can coordinately induce the expression of co-targeted genes. Th17 and Th0 differentiated T cells were transduced with IRF4 and/or BATF expressing retroviruses and assessed surface expression of ICOS and intracellular expression of CTLA-4 by flow cytometry. Over-expression of IRF4 and BATF alone induces the expression of ICOS in Th0 differentiated cells (FIG. 11a). However, co-expression of IRF-4 and BATF significantly increases ICOS levels, about 6 fold higher than seen for IRF4 and BATF alone (FIG. 11a, b). Similarly, CTLA-4 levels were slightly evaluated by IRF4 or BATF over-expression, but induced more significantly (3-fold) by co-expression of IRF4 and BATF (FIG. 11a-b). Consistent with these findings ICOS and CTLA-4 mRNAs were more highly induced co-expression of IRF-4 and BATF (data not shown).

### Example 8

### Co-Expression of IRF-4 and BATF Enhances the Generation of Th17 Cells

To test the functionality of molecular complexes between IRF-4 and BATF, gain-of-function experiments were performed to determine if their co-expression could augment the activation of select target genes containing AICE motifs and the generation of Th17 cells. The activation of CTLA-4 expression was tested for by transducing nonpolarized CD4+ T cells with IRF-4 and/or BATF expressing retroviruses and then assessed intracellular CTLA-4 protein accumulation by flow cytometry. CTLA-4 induction was modestly elevated by IRF-4 or BATF over-expression, but more substantially by co-expression of IRF-4 and BATF (FIG. 8c, d). Expression of ICOS was similarly more highly induced by the co-expression of IRF-4 and BATF in activated T cells (FIG. 11a, b). To test whether IRF-4 and BATF can enhance the generation of Th17 cells, in-vitro differentiating Th17 cells were transduced with IRF-4 and/or BATF expressing retroviruses and enumerated IL-17A producing cells by intracellular flow cytometry. IRF-4 or BATF over-expression did not appreciably increase the frequency of IL-17A producing cells whereas their co-expression resulted in a two-fold enhancement (FIG. 8e, D. Thus elevated co-expression of IRF-4 and BATF augments the frequency of T cells that express IL-17.

### Example 9

### Specificity of IRF-4/AP-1 Complexes Assembling on AICE Motifs

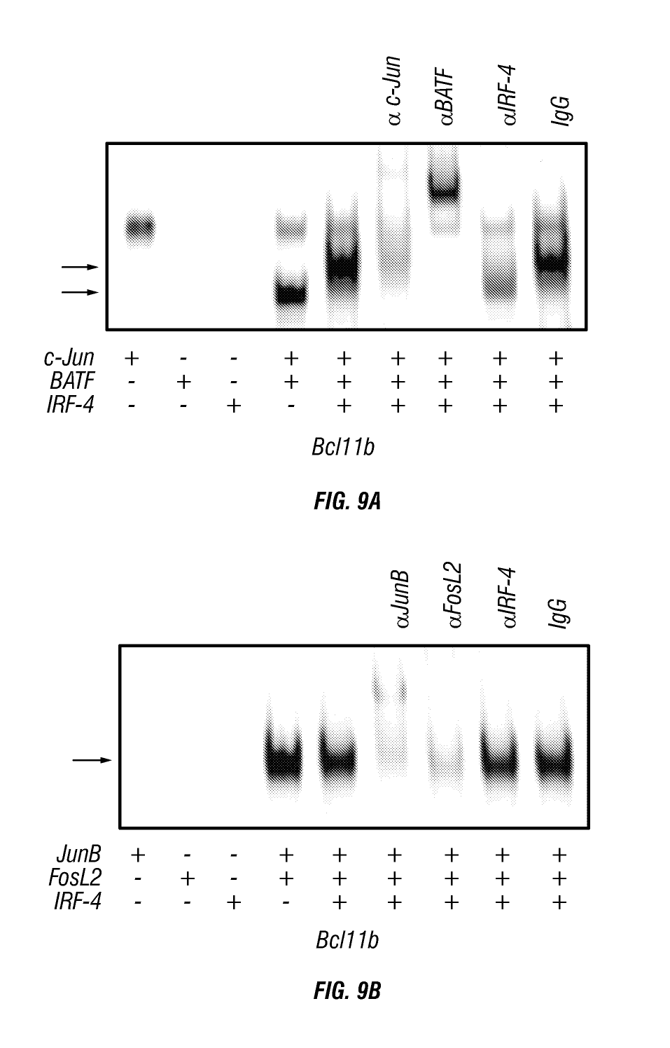

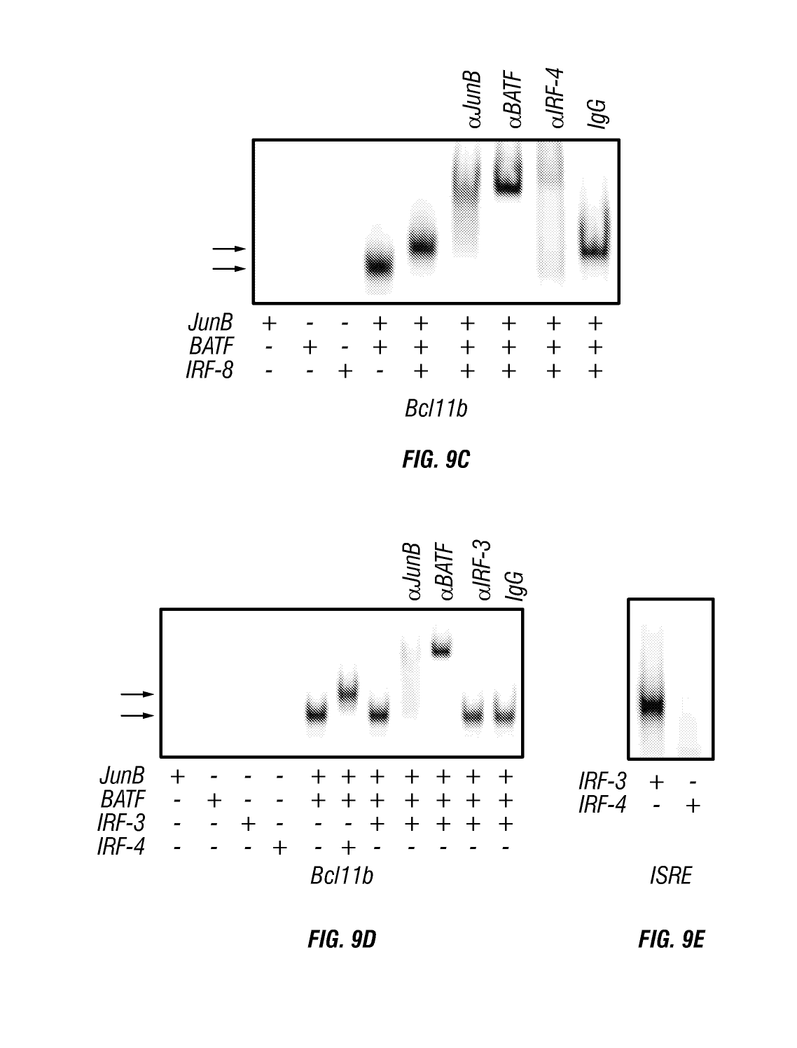

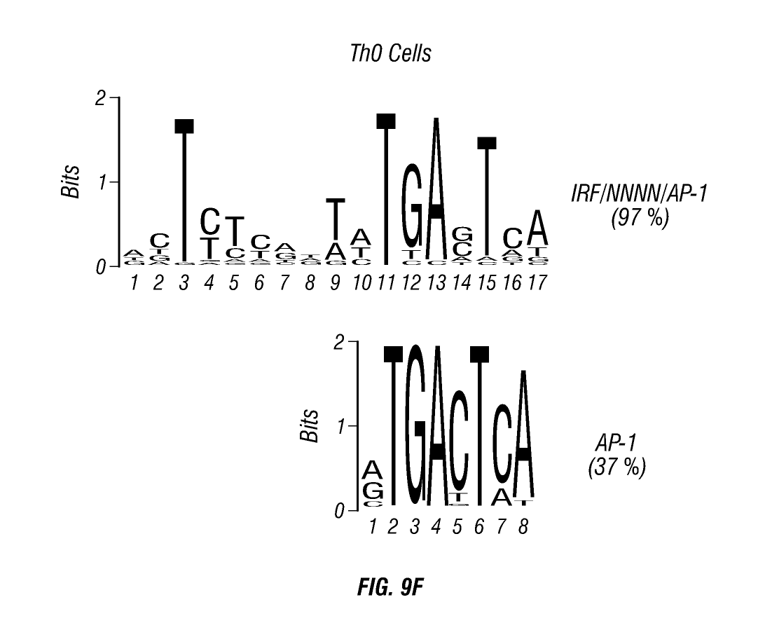

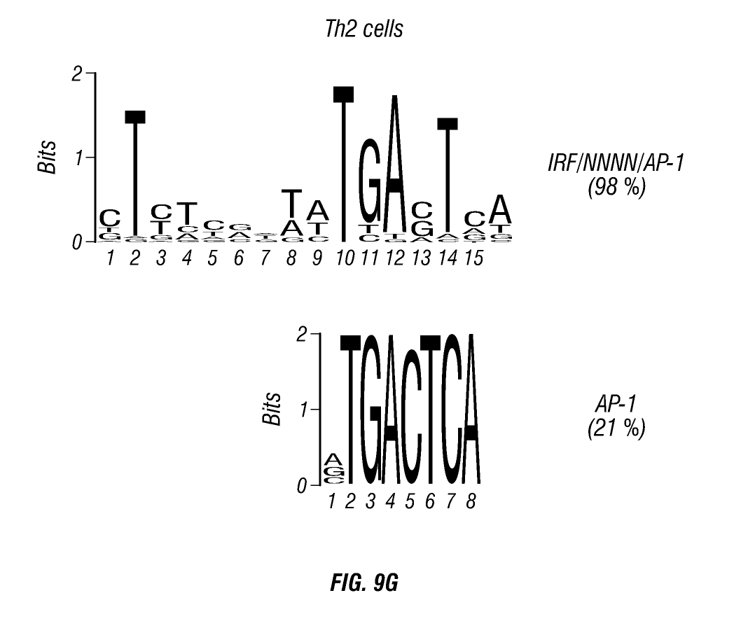

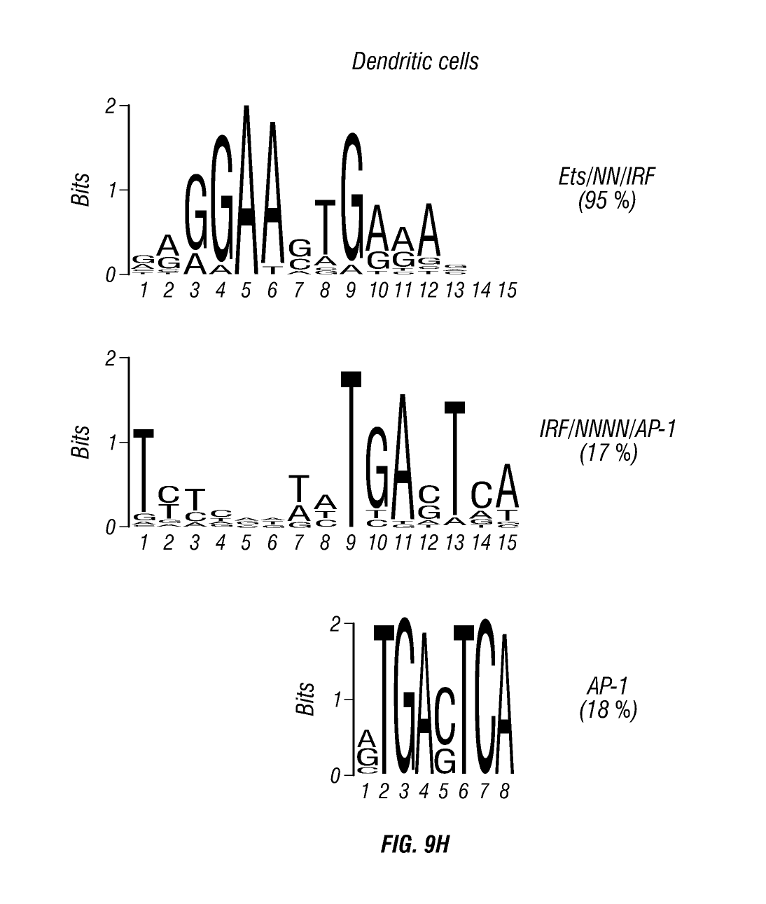

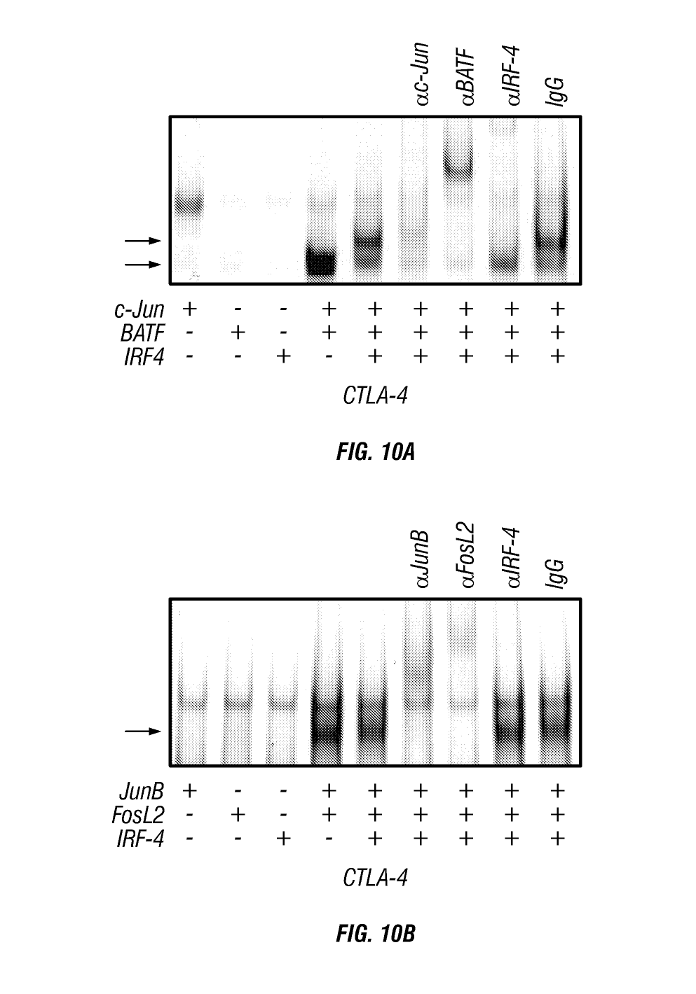

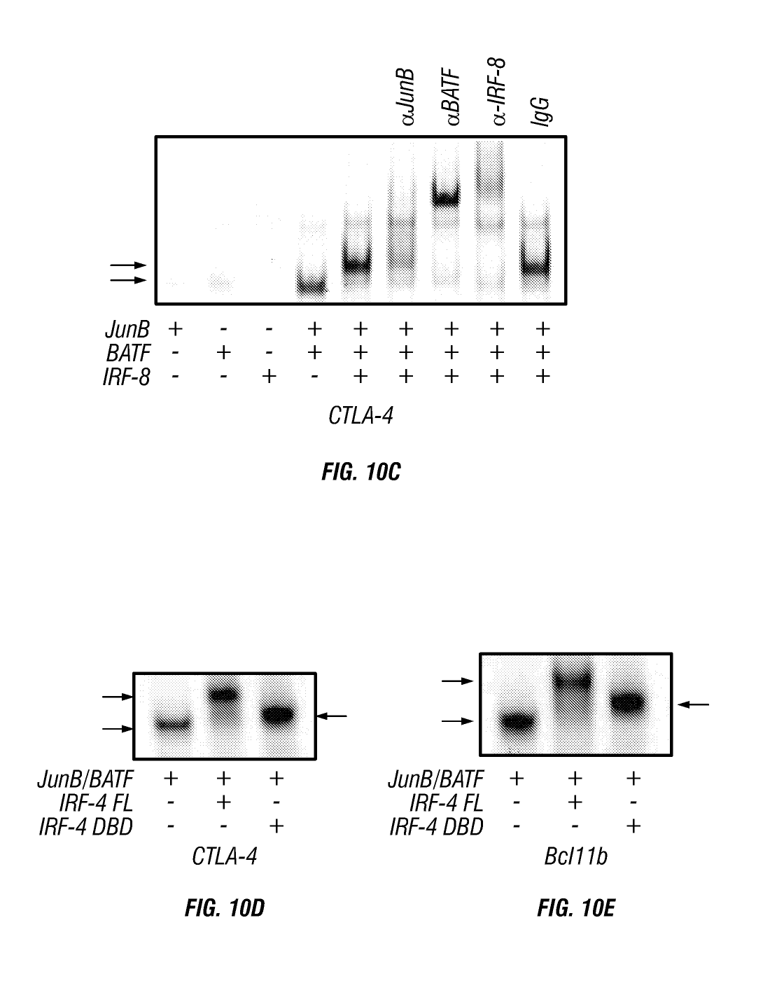

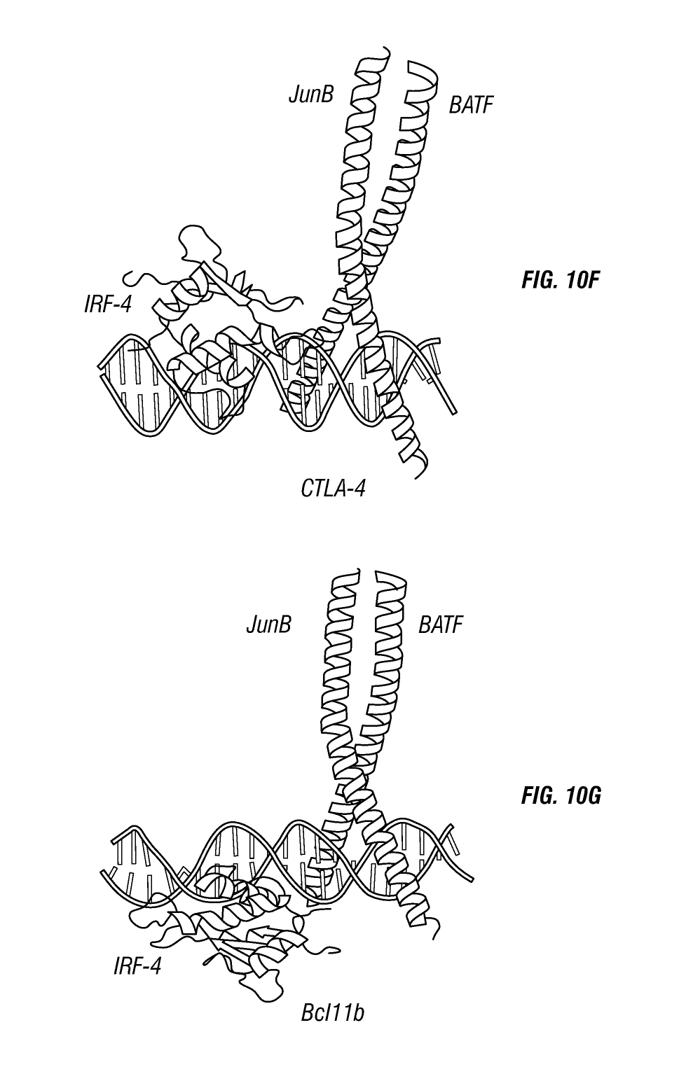

The AP-1 family of transcription factors consists of structurally related members belonging to the Jun, Fos, ATF and JDP sub-families (P. W. Vesely, P. B. Staber, G. Hoefler, L. Kenner, Mutation Research 682, 7 (2009)). These proteins share a conserved bZIP domain that consists of a leucine zipper and a basic domain responsible for dimerization and DNA binding, respectively. Expression profiling of AP-1 family genes in Th17 cells not only revealed the inducible expression of BATF and JunB but also that of c-Jun and FosL2 (data not shown). Therefore, it was tested whether alternate AP-1 complexes containing these subunits were capable of cooperatively assembling with IRF-4 on AICE motifs. Notably, a c-Jun/BATF heterodimer was also able to recruit IRF-4 to the structurally distinct AICE motifs (FIG. 9a and FIG. 10a). Surprisingly, although JunB/FosL2 heterodimers could recognize the AP-1 sites in the AICE motif, they were unable to recruit IRF-4 (FIG. 9b and FIG. 10b). Thus BATF heterodimers comprising either JunB or c-Jun can cooperatively assemble with IRF-4 on AICE motifs. AP-1 complexes comprised of Jun/Fos subunits appear to lack this distinctive molecular property and would be predicted to compete with the former for binding to AICE motifs.

Given the high degree of structural similarity of IRF-4 with IRF-8 it was determined if the latter factor could also be recruited to AICE motifs by BATF/Jun heterodimers. IRF-8 was seen to cooperatively assemble on AICE motifs with BATF/JunB heterodimers (FIG. 9c and FIG. 10c). However this molecular property was not manifested by a ubiquitously expressed IRF, namely IRF-3. It not only failed to recognize the AICE motif on its own but was not recruited to the DNA via a BATF/JunB complex (FIG. 9d). It is noted that IRF-3 unlike IRF-4 could bind with high affinity to an ISRE DNA probe that contained a dimeric GAAA core sequence (FIG. 9e). Thus IRF-4 and -8 are distinguished from other IRFs not only by their immune system restricted expression but also by their unique molecular properties of cooperative assembly with select members of the Ets and AP-1 superfamilies on EICE and AICE motifs, respectively.

To examine the structural basis of assembly of AP-1/IRF-4 complexes on the two structurally distinct AICE motifs, it was tested whether the DBD of IRF-4 could be recruited to DNA by BATF/JunB complexes. This was indeed the case (FIG. 10d, e) and rather unexpected based on molecular models of the DBD of IRF-4 and that of the BATF/JunB heterodimer docked onto the distinctive AICE motifs (FIG. 10f, g). As observed in the molecular models, the introduction of a 4 bp spacing between the AP-1 and IRF sites along with an inversion of the latter site results in dramatic spatial re-configuration of the IRF-4 DBD in relation to the DBD of the BATF/JunB heterodimer. It should be noted that these results do not imply that the DBDs of IRF-4 and the BATF/JunB heterodimer are sufficient to mediate cooperative assembly. However, they do suggest an unusually pliant mode of cooperative DNA binding that enables IRF-4 to assemble on structurally distinctive AICE motifs. X-ray crystallography along with mutational analysis of relevant protein interaction surfaces will be needed to address the basis of this exceptional molecular property shared by IRF-4 and -8.

Given widespread distribution of AP-1 family members and that IRF-4 is expressed in an inducible manner in various T helper cells as well as in B, macrophage and dendritic cells, ChIPseq was utilized to determine if IRF-4 is able to target AICE motifs in alternate cellular contexts. The IRF-4 cistromes were analyzed in activated but non-polarized T cells (Th0), IL-4 polarized Th2 cells and LPS activated dendritic cells. All three cellular contexts revealed enrichment for the AICE motif in targeted sequences (FIG. 9f-h). It should be noted that since dendritic cells express PU.1 and Spi-B whereas Th0 and Th2 cells do not, the EICE motif was observed within the IRF-4 cistrome in the former context but was absent in the latter. As is the case for dendritic cells, the IRF-4 cistrome in B cells was also seen to contain EICE as well as AICE motif targeted regions (R. Sciammas and H. Singh, unpublished data). These results suggest that IRF-4 assembles with AP-1 family factors on the AICE motif in both adaptive and innate immune cell contexts.

### REFERENCES

Betz, B. C., K. L. Jordan-Williams, et al. (2010). “Batf coordinates multiple aspects of B and T cell function required for normal antibody responses.” J Exp Med 207(5): 933-42.
Brass, A. L., A. Q. Zhu, et al. (1999). “Assembly requirements of PU.1-Pip (IRF-4) activator complexes: inhibiting function in vivo using fused dimers.” EMBO J 18(4): 977-91.
Brustle, A., S. Heink, et al. (2007). “The development of inflammatory T(H)-17 cells requires interferon-regulatory factor 4.” Nat Immunol 8(9): 958-66.
DeKoter, R. P., H. J. Lee, et al. (2002). “PU.1 regulates expression of the interleukin-7 receptor in lymphoid progenitors.” Immunity 16(2): 297-309.
Eisenbeis, C. F., H. Singh, et al. (1995). “Pip, a novel IRF family member, is a lymphoid-specific, PU.1-dependent transcriptional activator.” Genes Dev 9(11): 1377-87.
Escalante, C. R., A. L. Brass, et al. (2002). “Crystal structure of PU.1/IRF-4/DNA ternary complex.” Mol Cell 10(5): 1097-105.
Hirahara, K., K. Ghoreschi, et al. (2010). “Signal transduction pathways and transcriptional regulation in Th17 cell differentiation.” Cytokine Growth Factor Rev 21(6): 425-34.
Huber, M., A. Brustle, et al. (2008). “IRF4 is essential for IL-21-mediated induction, amplification, and stabilization of the Th17 phenotype.” Proc Natl Acad Sci USA 105(52): 20846-51.
Klein, U., S. Casola, et al. (2006). “Transcription factor IRF4 controls plasma cell differentiation and class-switch recombination.” Nat Immunol 7(7): 773-82.
Lohoff, M., H. W. Mittrucker, et al. (2002). “Dysregulated T helper cell differentiation in the absence of interferon regulatory factor 4.” Proc Natl Acad Sci USA 99(18): 11808-12.
Mudter, J., J. Yu, et al. (2011). “IRF4 regulates IL-17A promoter activity and controls RORgammat-dependent Th17 colitis in vivo.” Inflamm Bowel Dis 17(6): 1343-58.
Nagulapalli, S, and M. L. Atchison (1998). “Transcription factor Pip can enhance DNA binding by E47, leading to transcriptional synergy involving multiple protein domains.” Mol Cell Biol 18(8): 4639-50.
Schraml, B. U., K. Hildner, et al. (2009). “The AP-1 transcription factor Batf controls T(H)17 differentiation.” Nature 460(7253): 405-9.
Sciammas, R., A. L. Shaffer, et al. (2006). “Graded expression of interferon regulatory factor-4 coordinates isotype switching with plasma cell differentiation.” Immunity 25(2): 225-36.
Zhou, L. and D. R. Littman (2009). “Transcriptional regulatory networks in Th17 cell differentiation.” Curr Opin Immunol 21(2): 146-52.

1. An isolated molecular complex comprising IRF-4 and an AP-1 family member.

2. The isolated molecular complex of claim 1, wherein the AP-1 family member is BATF.

3. The isolated molecular complex of claim 2, further comprising an additional AP-1 family member.

4. The isolated molecular complex of claim 3, wherein the additional AP-1 family member is JunB.

5. The isolated molecular complex of claim 3, wherein the additional AP-1 family member is c-Jun.

6. The isolated molecular complex of any one of claims claim 1-5, further comprising a DNA sequence comprising an AP-1/IRF composite motif.

7. The isolated molecular complex of claim 6, wherein the DNA sequence is derived from a gene targeted by the IRF-4 and AP-1 complex.

8. The isolated molecular complex of claim 1, wherein the complex is capable of binding an AP-1/IRF composite motif comprising an IRF site and an AP-1 site.

9. The isolated molecular complex of claim 8, wherein the AP-1/IRF composite motif comprises a sequence provided in FIG. 1C.

10. The isolated molecular complex of claim 9, wherein the AP-1/IRF composite motif comprises a 4-bp space between the IRF site and the AP-1 site.

11. The isolated molecular complex of claim 10, wherein the AP-1/IRF composite motif comprises the sequence TTTC(N4)TGA(G/C)T(C/A)A (SEQ ID NO: 3).

12. The isolated molecular complex of claim 8, wherein the AP-1/IRF composite motif comprises no space between the IRF site and the AP-1 site.

13. The isolated molecular complex of claim 12, wherein the AP-1/IRF composite motif comprises the sequence GAAATGA(G/C)T(C/A)A (SEQ ID NO: 4).

14. The isolated molecular complex of claim 8, wherein the IRF site is TTTC and the AP-1 site is TGA(C/G)TCA.

15. The isolated molecular complex of claim 8, wherein the complex upon binding the AP-1/IRF composite motif is capable of inducing expression of T helper cell genes.

16. The isolated molecular complex of claim 15, wherein the T helper cell gene is CTLA-4.

17. The isolated molecular complex of claim 15, wherein the T helper cell gene is ICOS.

18. The isolated molecular complex of claim 8, wherein the complex upon binding the AP-1/IRF composite motif is capable of regulating genes listed in FIG. 7A, FIG. 7C, FIG. 8A, or FIG. 8B.

19. The isolated molecular complex of claim 8, wherein the complex upon binding the AP-1/IRF composite motif is capable of inducing the differentiation of Th17 cells.

20. The isolated molecular complex of claim 8, wherein the complex upon binding the AP-1/IRF composite motif is capable of increasing the frequency of 1L-17A-producing cells.

21. A method for identifying an agent characterized by the ability to inhibit IRF-4/AP-1 family member interaction, said method comprising:
a) incubating a reaction mixture comprising a candidate agent to be screened for the ability to inhibit IRF-4/AP-1 family member interaction, and a mixture of IRF-4 or an active fragment thereof and AP-1 family member or an active fragment thereof for a period of time and under conditions sufficient for IRF-4/AP-1 family member interaction; and
b) determining the extent of IRF-4/AP-1 family member interaction relative to an otherwise identical reaction mixture which does not include said candidate agent, wherein a decrease in the interaction relative to that of the otherwise identical reaction mixture is indicative of said candidate agent having the ability to inhibit IRF-4/AP-1 family member interaction.

22. The method of claim 21, wherein the AP-1 family member is BATF.

23. The method of claim 21 wherein the IRF-4/AP-1 family member interaction is detected by electromobility shift assay (EMSA).

24. The method of claim 21, wherein the agent is a small molecule.

25. The method of claim 21, wherein the agent is a peptidomimetic.

26. A method of treating a Th17-mediated disease comprising administering to a subject in need an agent identified by the method of claim 21.

27. A method of treating a Th17-mediated disease comprising administering to a subject in need an agent that inhibits the interaction of IRF-4 and an AP-1 family member.

28. The method of claim 27, wherein the AP-1 family member is BATF.

29. The method of claim 27, wherein the Th17 mediated disease is selected from the group consisting of multiple sclerosis, rheumatoid arthritis, inflammatory bowel disease, and psoriasis.

30. The method of claim 26 or claim 27, wherein the subject is a human patient.

31. An agent identified by the method of claim 21.

32. The agent of claim 31, wherein the agent is a small molecule.

33. The agent of claim 31, wherein the agent is a peptidomimetic.

34. A method of inhibiting the recruitment of IRF-4 to an AP-1/IRF composite motif by a DNA-bound BATF/JunB heterodimer.

35. A method of inhibiting the recruitment of IRF-4 to an AP-1/IRF composite motif by a DNA-bound BATF/c-Jun heterodimer.

36. An isolated molecular complex comprising IRF-8 and an AP-1 family member.

37. The isolated molecular complex of claim 36, wherein the AP-1 family member is BATF.

38. The isolated molecular complex of claim 36 or 37, further comprising an additional AP-1 family member.

39. The isolated molecular complex of claim 38, wherein the additional AP-1 family member is JunB.

40. The isolated molecular complex claim 36, further comprising a DNA sequence comprising an AP-1/IRF composite motif.

41. The isolated molecular complex of claim 40, wherein the DNA sequence is derived from a gene targeted by the IRF-4 and AP-1 complex.

42. The isolated molecular complex of claim 36, wherein the complex is capable of binding an AP-1/IRF composite motif comprising an IRF site and an AP-1 site.

43. The isolated molecular complex of claim 42, wherein the AP-1/IRF composite motif comprises a sequence provided in FIG. 1C.

44. The isolated molecular complex of claim 42, wherein the AP-1/IRF composite motif comprises a 4-bp space between the IRF site and the AP-1 site.

45. The isolated molecular complex of claim 44, wherein the AP-1/IRF composite motif comprises the sequence TTTC(N4)TGA(G/C)T(C/A)A (SEQ ID NO: 3).

46. The isolated molecular complex of claim 42, wherein the AP-1/IRF composite motif comprises no space between the IRF site and the AP-1 site.

47. The isolated molecular complex of claim 46, wherein the AP-1/IRF composite motif comprises the sequence GAAATGA(G/C)T(C/A)A (SEQ ID NO: 4).

48. The isolated molecular complex of any one of claims 40-46, wherein the IRF site is TTTC and the AP-1 site is TGA(C/G)TCA.

49. A method of manufacturing an agent capable of inhibiting the IRF4 and AP-1 family member interaction.
